(19) United States
(12) Patent Application Publication
(10) Pub. No.: US 2019/0000940 A1
(43) Pub. Date: Jan. 3, 2019

(54) COMPOSITIONS AND METHODS FOR THE TREATMENT OF AADC DEFICIENCY

(71) Applicant: Voyager Therapeutics, Inc., Cambridge, MA (US)

(72) Inventors: Robert Kotin, Boston, MA (US); Adrian Philip Kells, Arlington, MA (US); Bernard Ravina, Cambridge, MA (US)

(21) Appl. No.: 15/749,019

(22) PCT Filed: Jul. 29, 2016
(86) PCT No.: PCT/US16/44627

§ 371 Date: Jan. 30, 2018

Related U.S. Application Data
(60) Provisional application No. 62/334,604, filed on May 11, 2016.
(60) Provisional application No. 62/243,545, filed on Oct. 19, 2015.
(60) Provisional application No. 62/199,592, filed on Jul. 31, 2015.

(51) Int. Cl. A61K 38/51; A61K 48/00; A61P 25/00

(52) U.S. Cl. CPC A61K 38/51; A61P 25/00; C12Y 401/01028; A61K 48/005

(57) ABSTRACT

The invention relates to compositions and methods for the preparation, manufacture and therapeutic use of polynucleotides encoding AADC for the treatment, prophylaxis, palliation or amelioration of diseases, conditions and/or symptoms resulting from AADC deficiency and related inborn errors of neurotransmitter metabolism.

### CROSS REFERENCE TO RELATED APPLICATIONS

This application claims the benefit of U.S. Provisional Patent Application No. 62/199,592, entitled Compositions and Methods for the Treatment of AADC Deficiency, filed Jul. 31, 2015, U.S. Provisional Patent Application No. 62/243,545 entitled Compositions and Methods for the Treatment of AADC Deficiency, filed Oct. 19, 2015, U.S. Provisional Patent Application No. 62/334,604, entitled Compositions and Methods for the Treatment of AADC Deficiency, filed May 11, 2016; the contents of each of which are herein incorporated by reference in their entirety.

### REFERENCE TO SEQUENCE LISTING

The present application is being filed along with a Sequence Listing in electronic format. The Sequence Listing file, entitled 2057_1024PCT_SEQLST.txt, was created on Jul. 29, 2016 and is 4,405,208 bytes in size. The information in electronic format of the Sequence Listing is incorporated herein by reference in its entirety.

### FIELD OF THE INVENTION

The present disclosure relates to compositions, particularly nucleic acid molecules, e.g., polynucleotides encoding aromatic L-amino acid decarboxylase (AADC), for use in the treatment of AADC Deficiency (AADCD) and related childhood monoamine neurotransmitter disorders. In some embodiments polynucleotides may be encoded by or within recombinant adeno-associated viruses (AAVs).

### BACKGROUND OF THE INVENTION

Childhood neurotransmitter disorders are increasingly recognized as an expanding group of inherited neurometabolic syndromes. They are caused by disturbance in synthesis, metabolism, and homeostasis of the monoamine neurotransmitters, including the catecholamines (dopamine, norepinephrine, and epinephrine) and serotonin. Early and accurate diagnosis of childhood neurotransmitter disorders is important, as many are amenable to therapeutic intervention. Principles of treatment for childhood monoamine neurotransmitter disorders are based on an understanding of the relevant metabolic pathways. Most monoamine neurotransmitter disorders lead to reduced levels of central dopamine and/or serotonin, and monoamine neurotransmitter disorders associated with abnormal dopamine metabolism are associated with predominantly neurological phenotypes. Complete amelioration of motor symptoms is achievable in some disorders, such as Segawa's syndrome, and, in other conditions, significant improvement in quality of life can be attained with pharmacotherapy (Ng, et al., 2014, Pediatr. Drugs (2014) 16:275-291).

Defects in the AADC gene cause aromatic L-amino-acid decarboxylase deficiency (AADCD). AADCD is a progressive and debilitating neurodegenerative disease of the CNS arising as an inborn error in neurotransmitter metabolism. Thus, the disease almost always first appears at birth or early infancy. Because the AADC gene is involved in the synthesis of the neurotransmitters dopamine and serotonin, children with AADCD exhibit developmental delays, and severe neurologic dysfunction, including motor, sensory as well as involuntary muscle dysfunctions. Other clinical manifestations of AADCD include muscular hypotonia, dystonia, hypokinesia, athetosis, chorea, oculogyric crises, feeding or speech difficulty, insomnia, irritability, cyanosis, and various signs of autonomic dysfunction such as excessive sweating or temperature instability, beginning in infancy and early childhood (Brun et al., 2010, Neurology, 75(1):64-71). Response to current treatments is often disappointing.

In general, finding successful treatments for neurodegenerative diseases is particularly difficult due to the post-mitotic nature of central nervous system (CNS) neurons and the limited ability of neuronal cells to regenerate. Furthermore, because the blood-brain barrier (BBB) impedes peripheral access to the brain, there are inherent limitations on delivery of protein and peptide based therapeutics. The mature BBB serves as a protective barrier that excludes potentially damaging molecules and microorganisms based on size, charge, and lipid solubility. Consequently, the mature BBB efficiently blocks rAAV diffusion into the CNS. Thus, historically, to efficiently target the CNS using rAAV, researchers have had to resort to direct intraparenchymal injections (Manfredsson, et al., 2009, “AAV9: a potential blood-brain barrier buster.” Molecular Therapy 17(3):403-405). Overall, gene therapeutic approaches allowing the delivery of genes engineered for efficient CNS expression may be the most promising platform for treatment, prophylaxis and/or amelioration of CNS neurodegenerative disorders. (Feng and Maguire-Zeiss, 2010, CNS Drugs 24(3):177-192).

Because pharmacological treatment has only marginal effects on some of the symptoms and does not change early childhood mortality, gene therapy is the strategy of choice for treating AADCD. However, the current gene therapy for AADC deficiency only targets the putamen, leaving other areas in the brain and body untreated (Hwu, W. L., et al., 2012. Gene therapy for aromatic L-amino acid decarboxylase deficiency. Sci. Transl. Med. Vol. 4, 134ra61). Another shortcoming of some studies is that AAV2 does not undergo retrograde axonal transport in the brain and putaminally-infused vector would not be expected to transduce affected catecholaminergic neurons with any appreciable efficiency. Furthermore, anterograde transport of AAV2 and its payload after putaminal infusion resulted in AADC expression in many non-targeted nuclei such as globus pallidus, subthalamic nucleus and substantia nigra pars reticulata (SNpr).

Thus, a long-felt need remains for gene therapies for the treatment, amelioration, palliation or prophylaxis of AADCD and related inborn errors of neurotransmitter metabolism; such therapies may augment the diminished neurotransmitter(s), by directly or indirectly increasing their production, extending their half-life and/or decreasing their metabolism.

The adeno-associated virus (AAV) is a member of the parvovirus family and has emerged as an attractive vector for gene therapy in large part because this virus is apparently non-pathogenic; in fact, AAV has not been associated with any human disease. Further appeal is due to its ability to transduce dividing and non-diving cells (including efficient transduction of neurons), diminished proinflammatory and immune responses in humans, its inability to autonomously replicate without a helper virus (AAV is a helper-dependent DNA parvovirus which belongs to the genus Dependovirus), and its long-term gene expression. Although over 10 recombinant AAV serotypes (rAAV) have been engineered into vectors, rAAV2 is the most frequently employed serotype for gene therapy trials. Additional rAAV serotypes have been developed and tested in animal models that are more efficient at neuronal transduction.

The present disclosure provides improved nucleic acid constructs, e.g., polynucleotides, for use with AAV-derived vectors comprising the polynucleotides encoding aromatic L-amino acid decarboxylase (AADC) gene sequence (also known as “DOPA decarboxylase” or “DDC” and identified as gene/locus MIM number 107930 on chromosome 7p12) which encodes a full-length AADC protein for the purpose of gene therapy in the treatment of AADCD.

### SUMMARY OF THE INVENTION

Described herein are methods, processes, compositions kits and devices for the administration of AADC polynucleotides encoded by or contained within recombinant adeno-associated viruses (AAV) or viral particles for the treatment, prophylaxis, palliation and/or amelioration of AADCD and related inborn errors of neurotransmitter metabolism.

In one aspect, the present invention provides a method of treating AADC deficiency or a related childhood monoamine neurotransmitter disorder by administering a composition which may have at least one AAV particle, to the substantia nigra pars compacta of a subject in need of treatment or amelioration of said disorder. In one aspect, the subject in need of treatment is a human between 2 and 8 years old. The AAV particle of the invention may include an AADC polynucleotide with at least 95% identity to SEQ ID NOs 2-24. In one aspect, the AADC polynucleotide may have 99% identity to SEQ ID NOs 2-24.

In another aspect, the present invention provides a method of treating AADC deficiency or a related childhood monoamine neurotransmitter disorder by administering a composition which may have at least one AAV particle, to the putamen of a subject in need of treatment or amelioration of said disorder. The AAV particle of the invention may include an AADC polynucleotide with at least 95% identity to SEQ ID NOs 2-24.

In another aspect, the present invention provides a method of improving the sleep-wake cycle of a subject by administering to the subject a composition which may include at least one AAV particle to the putamen of a subject in need of treatment or amelioration of said disorder, wherein the AAV particle may include an AADC polynucleotide such as, but not limited to, SEQ ID NOs 2-24 or variants having at least 95% identity thereto.

In another aspect, the present invention provides a method of treating a sleep disorder by administering to the subject a composition which may include at least one AAV particle to the putamen of a subject in need of treatment or amelioration of said disorder, wherein the AAV particle may include an AADC polynucleotide such as, but not limited to, SEQ ID NOs 2-24 or variants having at least 95% identity thereto.

The AAV particle of the present invention may include a capsid serotype such as, but not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy. 5, AAVCy.5R1, AAVCy. 5R2, AAVCy.5R3, AAVCy. 5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, or any variant thereof. In one aspect, the AAV serotype may be any of the AAV serotypes listed in Table 1, or any variant thereof. In one aspect the AAV serotype may be, but is not limited to, AAV6, AAVrh10, AAV9 (hu14), AAV-DJ and AAV9.47. The AAV particle which may include any of these serotypes described above, may be used for any of the methods described herein.

The AAV particle of the present invention may include an AADC polynucleotide, and the AADC polynucleotide may include a promoter region, 5′ UTR, 3′ UTR and a poly(A) signal. In one aspect the AADC polynucleotide may include at least one 5′ ITR and one 3′ ITR. In one aspect, one or more 5′ ITRs are located 5′ to the promoter region and one or more of the 3′ ITRs are located at the 3′ end of the poly(A) signal. In one aspect the promoter region may include a promoter region which may include an enhancer element, a promoter element, a first exon region, a first intron region, a second intron region and a second exon region. In one aspect, the enhancer element and the promoter element are derived from cytomegalovirus (CMV). In one aspect, wherein the first exon region is ie1 exon 1 or fragments thereof, the first intron region is ie1 intron 1 or fragments thereof, the second intron region is hβglobin intron 2 or fragments thereof and the second exon region is hβglobin exon 3 or fragments thereof. In one aspect, the poly(A) signal is derived from human growth hormone. The AAV particle which may include any of AADC polynucleotides described above, may be used for any of the methods described herein.

Any of the methods described herein may utilize a composition which includes a ratio of at least 70:30 AAV particle which may include the AADC polynucleotide to AAV particles without the AADC polynucleotide. In one aspect, the ratio of AAV particle which may include AADC polynucleotide to AAV particle without AADC polynucleotide is at least 85:15. In another aspect, the ratio of AAV particle which may include an AADC polynucleotide to AAV particle without AADC polynucleotide is 100:0.

In any of the methods described herein, during the administration of the composition to a subject in need thereof, the subject may use a compression device during the contacting of the subject with the composition. In one aspect, the compression device may reduce the likelihood of deep vein thrombosis in the subject.

The details of various embodiments of the disclosure are set forth in the description below. Other features, objects, and advantages of the disclosure will be apparent from the description and from the claims.

### DETAILED DESCRIPTION

A brief description of the AADC gene and neurotransmitter synthesis: Dopamine is the precursor of catecholaminergic hormones and is also itself a neurotransmitter in the basal ganglia. Dopamine is involved in many processes in the CNS, including motor function, cognition, mood, REM sleep and even certain behaviors. At least five types of dopamine receptors occur throughout the body and are responsible for various effects of the neurotransmitter. The cell bodies of dopamine-producing neurons are found in a region of the brain called the substantia nigra pars compacta (SNpc), and the neurons project to a part of the brain called the striatum. Overall, in the brain, dopamine is transmitted along five paths including (1) the mesolimbic-mesocortical pathway, projecting from the substantia nigra to the limbic system and neocortex, involved in the reward system and implicated in addiction; (2) the nigrostriatal pathway, which projects from the substantia nigra to the striatum and is particularly involved in producing movement; (3) the tuberoinfundibular system in which nuclei in the hypothalamus produce and release dopamine into the portal circulation of the pituitary gland to inhibit secretion of prolactin from the anterior pituitary; (4) the medullary-periventricular pathway, which consists of neurons in the motor portion of the vagus nerve, and which pathway is suspected in eating behavior; and (5) the incertohypothalamic pathway, which may play a role in the anticipatory phase of sexual interaction. Non-striatal dopamine is also made by proximal tubule cells of the kidney where it is thought to act locally.

Dopamine is largely synthesized from the amino acid tyrosine in a series of enzymatic reactions initiated by the conversion of tyrosine to L-dopa by tyrosine hydroxylase and its co-factor tetrahydrobiopterin. L-dopa (a.k.a. levodopa) is then decarboxylated by aromatic L-amino acid decarboxylase (AADC) to form dopamine. AADC is a homodimeric pyridoxal phosphate-dependent enzyme involved in the synthesis of not only dopamine; it also decarboxylates L-5-hydroxytryptophan to serotonin and L-tryptophan to tryptamine. Dopamine is responsible for reward-driven learning, voluntary movement control, feeding, neuroendocrine secretion, cognition, and behavior. Serotonin has important neuromodulatory actions in cognitive, emotional, impulse control, circadian rhythm, sleep-wake cycle, pain, respiratory, and cardiovascular functions.

As an inborn error of metabolism, defects in the AADC gene result in a deficit of dopamine, serotonin and, manifesting in infancy as various and several neurological and developmental delays. In some cases of AADCD, biochemical symptoms include very low concentrations of homovanillic acid (HVA) and 5-hydroxy indole acetic acid (5-HIAA) in cerebrospinal fluid (CSF). Levels of 3-methoxy-4-hydroxyphenolglycole and norepinephrine may also be reduced. Also noted are a significant elevation in the urinary excretion of L-DOPA, 5-hydroxytryptophan (5HTP), and 3-methoxytyrosine, all of which precede the AADC enzymatic step in the biochemical pathway. AADC enzyme activity is severely reduced in plasma and in liver tissue (˜1% of control); thus, serotonin and dopamine synthesis are both affected in the central and peripheral nervous systems. About 24% of patients have an abnormal brain MRI, with cerebral atrophy, degenerative changes of the white matter, thinning of the corpus callosum, or a leukodystrophy-like pattern (Brun et al., 2010, Neurology, 75(1):64-71).

AADC deficiency has an increased prevalence in the Taiwanese population due to a founder mutation IVS6+4 A>T. In one report, 13 of the 16 mutated alleles (81.3%) in Taiwanese patients with AADC deficiency carried the IVS6+4 A>T mutation. Most Taiwanese patients with AADC deficiency present with oculogyric crisis and hypotonia before 1 year of age (H. F. Lee, et al., 2009, “Aromatic L-amino acid decarboxylase deficiency,” Taiwan. Eur. J. Paediatr. Neurol. 13:135-140).

Neurotransmitter replacement therapies (e.g., administration of levodopa) have been used to treat Parkinson's Disease (PD), a relatively common neurological disorder in which degeneration of a group of dopaminergic neurons in the midbrain leads to a deficiency of dopamine in the striatum. However, such pharmacotherapeutic techniques often yield diminishing benefits as the disease progresses and dopamine-generating cells die. Furthermore, systemic administration of high-dose dopamine is complicated by side effects, such as fluctuations in motor performance, dyskinesias, and hallucinations, resulting from dopaminergic stimulation of the mesolimbic system.

An early study in a rat model of PD showed that, upon adeno-associated virus (AAV)-AADC transduction, the transgenic AADC is able to decarboxylate exogenous L-DOPA more efficiently, such that a dose of L-DOPA previously ineffective before gene transfer was able to elicit a positive motor behavioral response following gene transfer. In animals receiving AAV2-AADC, dopamine production was restored to 50% of normal levels 12 weeks after the infusion. Microdialysis experiments demonstrated an in vivo enhanced conversion of L-DOPA to dopamine, but no storage capacity as dopamine was released to the extracellular space in a continuous, nonregulated fashion. It was suggested that, in addition to the potential clinical benefit of improving decarboxylation efficiency in Parkinson's disease, this approach might have some relevance for the treatment of AADC deficiency (Sanchez-Pernaute, et al., 2001, “Functional effect of adeno-associated virus mediated gene transfer of aromatic 1-amino acid decarboxylase into the striatum of 6-OHDA-lesioned rats.” Mol. Ther. 4:324-330).

In a primate model of PD, a mixture of three separate AAV vectors expressing tyrosine hydroxylase (TH), AADC, and GTP cyclohydrolase I (GCH) was stereotaxically injected into the unilateral putamen of 1-methyl-4-phenyl-1,2,3,6-tetrahydropyridine-treated monkeys, and coexpression of the enzymes in the unilateral putamen resulted in improvement in manual dexterity on the side contralateral to the AAV-TH/-AADC/-GCH-injected side, which persisted during the observation period (Muramatsu, et al., 2002, “Behavioral recovery in a primate model of Parkinson's disease by triple transduction of striatal cells with adeno-associated viral vectors expressing dopamine-synthesizing enzymes.” Hum. Gene Ther. 13: 345-354). Another study employing gene therapy to deliver AADC by intraputaminal infusion resulted in restored dopaminergic function and minimized side effects. The adeno-associated virus (AAV) serotype 2 vector, AAV-hAADC-2, was found to increase extracellular dopamine in the striatum after administration of levodopa (Muramatsu, et al., 2010, “A phase I study of aromatic L-amino acid decarboxylase gene therapy for Parkinson's disease.” Mol. Ther. 18:1731-1735; Forsayeth, J., et al., 2010, “Gene therapy for Parkinson's disease: Where are we now and where are we going?” Expert Review of Neurotherapeutics 10 (12): 1839). To treat the main symptoms of AADC-deficiency include motor impairment, a similar gene therapy approach has been suggested (Allen, et al., 2009, “A new perspective on the treatment of aromatic L-amino acid decarboxylase deficiency.” Mol. Genet. Metab. 97:6-14).

An AADC deficient murine model has been developed, by inserting an AADC gene mutation (IVS6+4A>T) and a neomycin-resistance gene into intron 6 of the mouse AADC (Ddc) gene. In the brains of homozygous knock-in (KI) mice (Ddc^{IVS6/IVS6}), AADC mRNA lacked exon 6, and AADC activity was <0.3% of that in wild-type mice. Half of the KI mice were born alive but grew poorly and exhibited severe dyskinesia and hind limb clasping after birth. Two-thirds of the live-born KI mice survived the weaning period, with subsequent improvements in their growth and motor functions; however, these mice still displayed cardiovascular dysfunction and behavioral problems due to serotonin deficiencies. The brain dopamine levels in the KI mice increased from 9.39% of the levels in wild-type mice at 2 weeks of age to 37.86% of the levels in wild-type mice at 8 weeks of age. Adult KI mice also exhibited an exaggerated response to apomorphine and an elevation of striatal c-Fos expression, suggesting post-synaptic adaptations; thus, compensatory regulation apparently allowed these mice to survive to adulthood (Lee, et al., 2013, Neurobiology of Disease 52:177-190).

In AADC-deficient children, a recent gene replacement clinical trial explored putaminal delivery of recombinant adeno-associated virus serotype 2 vector encoding human AADC (AAV2-hAADC). Unfortunately, the AADCD patients showed only modest amelioration of motor symptoms, hypothesized to be due to insufficient transduction of the putamen. With the development of a highly accurate MRI-guided cannula placement technology, a more effective approach targeted the affected mid-brain neurons directly. Transduction of AADC-deficient dopaminergic neurons in the substantia nigra with locally infused AAV2-hAADC was expected to lead to restoration of normal dopamine levels in affected children. The long-term safety and tolerability of bilateral AAV2-hAADC MRI-guided pressurized infusion into the mid-brain was also assessed in non-human primates; the animals received either vehicle, low or high AAV2-hAADC vector dose and were euthanized 1, 3 or 9 months after surgery, and effective mid-brain transduction was achieved without untoward effects (San Sebastian, et al., 2014, Mol. Ther. Methods Clin. Dev. 3: 14049).

Pharmacotherapeutic techniques have also been used to treat AADCD. Clinical management of AADCD usually involves pyridoxine (B6)/pyridoxal phosphate, dopamine agonists, and monoamine oxidase B inhibitors to potentiate monoaminergic transmission, in attempt to provide some improvement in tone and movement. Standard treatments for AADCD are: Pyridoxine 20-160 mg/kg/day; Folinic acid (calcium folinate) 15 mg/day [BNFC 2014]; Dopamine agonists: Rotigotine, patch dose as per BNFC; 2, 4, 6, 8 mg dose patches available and applied once a day (doses 0.17-0.25 mg/kg/day used) (other dopamine agonists used; pergolide, bromocriptine, pramipexole, ropinirole); MAOI selegiline 0.03-2 mg/kg/day; Trihexyphenydyl 1-12 mg/day titrated (often higher doses are tolerated but titrated slowly); Benztropine 1-4 mg/day; Clonidine 0.1-3 total mg/day (BNFC initial test dose is recommended with higher doses used with caution due to antihypertensive action); Benzodiazepines (Ng, et al., 2014, Pediatr. Drugs (2014) 16:275-291). However, in some cases, such treatments were found not to be very beneficial. Furthermore, in assessing treatment response among one group of AADC patients, a sex difference in the monoaminergic system was observed: a subgroup of five males responded to treatment and made developmental progress, while a second subgroup of five females and one male responded poorly to treatment and often developed drug-induced dyskinesias. Thus, females appeared more dependent on the dopamine system (Pons et al., 2004, Neurology 62:1058-1065).

In one embodiment, the nucleic acid constructs described herein may comprise at least a 5′-ITR and a 3′-ITR, each or both of which may be derived from an AAV, positioned about a DDC gene sequence, as well as additional components required for gene expression and clone selection.

### Compositions of the Invention

According to the present invention, AADC polynucleotides (specifically, novel SEQ ID Nos. 2 to 24) are provided which function alone or combinations thereof with additional nucleic acid sequence(s) to encode the AADC protein. As used herein an “AADC polynucleotide” is any nucleic acid which encodes an AADC protein and when present in a vector, plasmid or translatable construct, expresses such AADC protein in a cell, tissue, organ or organism.

AADC polynucleotides include precursor molecules which are processed inside the cell. AADC polynucleotides, or the processed forms thereof, may be encoded in a plasmid, vector, genome or other nucleic acid expression vector for delivery to a cell.

In some embodiments AADC polynucleotides are designed as components of AAV viral genomes and packaged in AAV viral particles which are processed within the cell to produce the wild type AADC protein.

As used herein, the wild type AADC protein may be any of the naturally occurring isoforms or variants from the DDC gene. Multiple alternatively spliced transcript variants encoding different isoforms of AADC have been identified. Specifically, the DDC gene produces seven transcript variants that encode six distinct isoforms. DDC transcript variants 1 and 2 both encode AADC isoform 1. In some embodiments, the AADC polynucleotides encode DDC transcript variant 2, thereby encoding a native AADC isoform 1 (NCBI Reference Sequence: NP_000781.1), which amino acid sequence is identified here as SEQ ID NO:1:

MNASEFRRRGKEMVDYVANYMEGIEGRQVYPDVEPGYLRPLIPAAAPQEP
DTFEDIINDVEKIIMPGVTHWHSPYFFAYFPTASSYPAMLADMLCGAIGC
IGFSWAASPACTELETVMMDWLGKMLELPKAFLNEKAGEGGGVIQGSASE
ATLVALLAARTKVIHRLQAASPELTQAAIMEKLVAYSSDQAHSSVERAGL
IGGVKLKAIPSDGNFAMRASALQEALERDKAAGLIPFFMVATLGTTTCCS
FDNLLEVGPICNKEDIWLHVDAAYAGSAFICPEFRHLLNGVEFADSFNFN
PHKWLLVNFDCSAMWVKKRTDLTGAFRLDPTYLKHSHQDSGLITDYRHWQ
IPLGRRFRSLKMWFVFRMYGVKGLQAYIRKHVQLSHEFESLVRQDPRFEI
CVEVILGLVCFRLKGSNKVNEALLQRINSAKKIHLVPCHLRDKFVLRFAI
CSRTVIESAHVQRAWEHIKELAADVLRAERE.

The AADC polynucleotides disclosed herein may be engineered to contain modular elements and/or sequence motifs assembled to create AADC polynucleotide constructs.

### AADC Polynucleotide Constructs

In some embodiments, the AADC polynucleotide comprises a sequence of any of SEQ ID NOs: 2 to 24, or a fragment thereof. Such polynucleotides comprise nucleic acids which comprise a region of linked nucleosides encoding one or more isoforms or variants of the AADC protein.

In some embodiments, the AADC polynucleotide comprises a codon optimized transcript encoding an AADC protein.

In one embodiment, the AADC polynucleotide comprises a sequence which has a percent identity to any of SEQ ID NOs: 2-24. The AADC polynucleotide may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ ID NOs: 2-24. The AADC polynucleotide may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NOs: 2-24. As a non-limiting example, the AADC polynucleotide comprises a sequence which has 80% identity to any of SEQ ID NOs 6, 7, 8, 9, 17, 18, 19, 20, 21, 22, 23 and 24. As another non-limiting example, the AADC polynucleotide comprises a sequence which has 85% identity to any of SEQ ID NOs 6, 7, 8, 9, 17, 18, 19, 20, 21, 22, 23 and 24. As another non-limiting example, the AADC polynucleotide comprises a sequence which has 90% identity to any of SEQ ID NOs 6, 7, 8, 9, 17, 18, 19, 20, 21, 22, 23 and 24. As another non-limiting example, the AADC polynucleotide comprises a sequence which has 95% identity to any of SEQ ID NOs 6, 7, 8, 9, 17, 18, 19, 20, 21, 22, 23 and 24. As another non-limiting example, the AADC polynucleotide comprises a sequence which has 99% identity to any of SEQ ID NOs 6, 7, 8, 9, 17, 18, 19, 20, 21, 22, 23 and 24. As a non-limiting example, the AADC polynucleotide comprises a sequence which has 80% identity to SEQ ID NO 24. As another non-limiting example, the AADC polynucleotide comprises a sequence which has 85% identity to SEQ ID NO 24. As another non-limiting example, the AADC polynucleotide comprises a sequence which has 90% identity to SEQ ID NO 24. As another non-limiting example, the AADC polynucleotide comprises a sequence which has 95% identity to SEQ ID NO 24. As another non-limiting example, the AADC polynucleotide comprises a sequence which has 99% identity to SEQ ID NO 24.

In some embodiments, the AADC polynucleotide comprises a sequence region encoding one or more wild type isoforms or variants of an AADC protein. Such polynucleotides may also comprise a sequence region encoding any one or more of the following: a 5′ ITR, a cytomegalovirus (CMV) Enhancer, a CMV Promoter, a synapsin 1 promoter, an ie1 exon 1, an ie1 intron1, intron 1 of human growth hormone, a P globin intron, an hβglobin intron2, an hβglobin exon 3, a 5′ UTR, a 3′ UTR, an hGH poly(A) signal, a simian virus 40 polyadenylation signal, a post-transcriptional regulatory element, a woodchuck hepatitis virus post-transcriptional regulatory element, and/or a 3′ ITR. Such sequence regions are taught herein or may be any of those known in the art.

In some embodiments the AADC coding region is 1440 nucleotides in length. Such an AADC polynucleotide may be codon optimized over all or a portion of the polynucleotide.

In some embodiments the AADC coding region is 1443 nucleotides in length. In such case, an additional codon may be present at the 3′ end of the polynucleotide.

In some embodiments the AADC coding region is 1449 nucleotides in length. In such case, additional codons may be present at the 3′ end of the polynucleotide.

In some embodiments, the AADC polynucleotide comprises any of SEQ ID NOs 13-24 but lacking the 5′ and/or 3′ ITRs. Such a polynucleotide may be incorporated into a plasmid or vector and utilized to express the encoded AADC protein.

In one embodiment, the AADC polynucleotide may comprise a codon optimized open reading frame of an AADC mRNA, at least one 5′ITR and at least one 3′UTR where the one or more of the 5′ITRs may be located at the 5′ end of the promoter region and one or more 3′ ITRs may be located at the 3′ end of the poly(A) signal. The AADC mRNA may comprise a promoter region, a 5′ untranslated region (UTR), a 3′UTR and a poly(A) signal. The promoter region may include, but is not limited to, enhancer element, a promoter element, a first exon region, a first intron region, a second intron region and a second exon region. As a non-limiting example, the enhancer element and the promoter element are derived from CMV. As another non-limiting example, the intron is derived from human growth factor. As another non-limiting example, the intron is derived from β globin. As another non-limiting example, the first exon region is iel exon 1 or fragments thereof, the first intron region is ie1 intron 1 or fragments thereof, the second intron region is hBglobin intron 2 or fragments thereof and the second exon region is hBglobin exon 3 or fragments thereof. As yet another non-limiting example, the poly(A) signal is derived from human growth hormone. As another non-limiting example, the poly(A) signal is derived from simian virus 40.

### Viral Vectors

The AADC polynucleotides disclosed herein can be introduced into host cells using any of a variety of approaches. Infection with a viral vector comprising the AADC polynucleotide can be effected. Examples of suitable viral vectors include replication defective retroviral vectors, adenoviral vectors, adeno-associated vectors and lentiviral vectors.

According to the present invention, viral vectors for use in therapeutics and/or diagnostics comprise a virus that has been distilled or reduced to the minimum components necessary for transduction of a nucleic acid payload or cargo of interest. In this manner, viral vectors are engineered as vehicles for specific delivery while lacking the deleterious replication and/or integration features found in wild-type virus.

As used herein, a “vector” is any molecule or moiety which transports, transduces or otherwise acts as a carrier of a heterologous molecule such as the polynucleotides described herein. A “viral vector” is a vector which comprises one or more polynucleotide regions encoding or comprising payload molecule of interest, e.g., a transgene, a polynucleotide encoding a polypeptide or multi-polypeptide.

### Viral Vectors: Adeno-Associated Virus (AAV)

Viral vectors of the present disclosure may be produced recombinantly and may be based on adeno-associated virus (AAV) parent or reference sequence. Serotypes which may be useful in the presently disclosed compositions and methods include any of those arising from AAV1, AAV2, AAV3, AAV4, AAV5, AAV6, AAV7, AAV8, AAV9, AAV9.47, AAV9(hu14), AAV10, AAV11, AAV12, AAVrh8, AAVrh10 and AAV-DJ.

AAV vectors may also comprise self-complementary AAV vectors (scAAVs). scAAV vectors contain both DNA strands which anneal together to form double stranded DNA. By skipping second strand synthesis, scAAVs allow for rapid expression in the cell.

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide or modulatory nucleic acid, which may be delivered to a target cell, tissue or organism.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

The Parvoviridae family comprises the Dependovirus genus which includes adeno-associated viruses (AAV) capable of replication in vertebrate hosts including, but not limited to, human, primate, bovine, canine, equine, and ovine species.

The AAV particles of the present disclosure comprise a nucleic acid sequence e.g., polynucleotide, encoding at least one “payload.” As used herein, a “payload” refers to one or more polynucleotides or polynucleotide regions encoded by or within a viral genome or an expression product of such polynucleotide or polynucleotide region e.g., a transgene, a polynucleotide encoding a polypeptide or multi polypeptide or a modulatory nucleic acid or regulatory nucleic acid. The term “AAV particle” or “AAV vector” as used herein comprises a capsid and a polynucleotide of payload. The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

The payload may comprise any nucleic acid known in the art which is useful for modulating the expression (by supplementation or gene replacement or by inhibition using a modulatory nucleic acid) in a target cell transduced or contacted with the AAV particle carrying the payload.

Vectors used in the production of AAV particles include those encoding the payload, e.g. payload construct vectors, and those encoding accessory proteins necessary for production, e.g. viral construct vectors.

According to the present invention, an AAV payload construct vector encodes a “payload construct.” In one embodiment, a “payload construct” is a polynucleotide sequence encoding at least a payload and sufficient ITR sequence to allow for replication thereby producing a viral genome that is packaged inside a capsid.

The payload construct may comprise a combination of coding and non-coding nucleic acid sequences.

In one embodiment, the AAV payload construct vector comprises more than one nucleic acid sequences encoding more than one payload of interest. In such an embodiment, a payload construct encoding more than one payload may be replicated and packaged into a viral particle. A target cell transduced with a viral particle comprising more than one payload may express each of the payloads in a single cell.

In some embodiments, the AAV payload construct may encode a coding or non-coding RNA.

Where the AAV payload construct sequence encodes a polypeptide, the polypeptide may be a peptide or protein. A protein encoded by the AAV payload construct sequence may comprise a secreted protein, an intracellular protein, an extracellular protein, and/or a membrane protein. The encoded proteins may be structural or functional. Proteins encoded by the payload construct vector or payload construct include, but are not limited to, mammalian proteins. The AAV polynucleotides encoding polypeptides (e.g., mRNA) described herein may be useful in the fields of human disease, antibodies, viruses, veterinary applications and a variety of in vivo and in vitro settings.

In some embodiments, the AAV particles are useful in the field of medicine for the treatment, prophylaxis, palliation or amelioration of AADCD and related childhood monoamine neurotransmitter disorders.

In some embodiments, the AAV payload construct encodes a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′UTR, a 3′UTR, a 5′ cap and a poly-A tail. According to the present invention, AAV payload constructs encoding mRNA may comprise a coding region only. They may also comprise a coding region and at least one UTR. They may also comprise a coding region, 3′UTR and polyA tail. In some embodiments, the mRNA or any portion of the AAV may be codon optimized.

### AAV Serotypes

In one embodiment, the AADC polynucleotide is encoded in a plasmid or vector, which may be derived from an adeno-associated virus (AAV). In one embodiment, the AAV serotype used in the present invention may be from a variety of species.

AAV particles of the present invention may comprise or be derived from any natural or recombinant AAV serotype. According to the present invention, the AAV particles may utilize or be based on a serotype selected from any of the following AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV 1-7/rh.48, AAV 1-8/rh.49, AAV2-15/rh. 62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

As a non-limiting example, the capsid of the recombinant AAV virus is AAV2. As a non-limiting example, the capsid of the recombinant AAV virus is AAVrh10. As a non-limiting example, the capsid of the recombinant AAV virus is AAV9(hu14). As a non-limiting example, the capsid of the recombinant AAV virus is AAV-DJ. As a non-limiting example, the capsid of the recombinant AAV virus is AAV9.47. As a non-limiting example, the capsid of the recombinant AAV virus is AAV-DJ8.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh. 13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6): 1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, the AAVDJ sequence described as SEQ ID NO: 1 in U.S. Pat. No. 7,588,772 may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of US9233131), AAVhEr2.4 (SEQ ID NO:54 of US9233131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of US9233131), AAVhEr2.36 (SEQ ID NO:57 of US9233131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

In one embodiment, the AAV serotype may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In one embodiment, the AAV serotype may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of US7427396), or variants thereof.

In one embodiment, the AAV serotype may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of US7427396), or variants thereof.

In other embodiments the AAV serotype may be engineered as a hybrid AAV from two or more parental serotypes. In one embodiment, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in their entirety.

In one embodiment, the AAV serotype may be a serotype generated by the AAV9 capsid library with mutations in amino acids 390-627 (VP1 numbering) as described by Pulicherla et al. (Molecular Therapy 19(6): 1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In some embodiments, the AAV serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: X and X of U.S. Pat. No. 8,734,809), AAV CLv-R9 (SEQ ID NO: X and X of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No. 8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ ID NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-H5 (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In one embodiment, the AAV particle may utilize or be based on a serotype comprising at least one AAV capsid CD8+ T-cell epitope. As a non-limiting example, the serotype may be AAV1, AAV2 or AAV8.

In one embodiment the AAV serotype may comprise an AAV9 capsid with a tyrosine to phenylalanine substitution at position 446 of the AAV9 capsid protein sequence, as described in Lee, N.C., et al., 2015 Benefits of neuronal preferential systemic gene therapy for neurotransmitter deficiency. Mol. Ther. Vol. 23(10), 1572-1581, the contents of which are herein incorporated by reference in their entirety.

In one embodiment, the AAV particle of the present invention may be an AAV2/9, wherein the ITRs are derived from AAV2 and the capsid from AAV9.

In one embodiment, the AAV particle of the present invention may be an AAV3/9, wherein the ITRs are derived from AAV3 and the capsid from AAV9.

The AAV particle of the present invention may comprise any combination of ITR and capsid to generate a pseudotyped virus.

In one embodiment, the AAV particle may utilize or be based on a serotype selected from any of those found in Table 1.

In one embodiment, the AAV particle may be generated as a hybrid or pseudotyped AAV from two or more serotypes described in Table 1. As a non-limiting example, the ITRs may be from a first serotype in Table 1 and the capsid may be from a second serotype in Table 1.

In one embodiment, the AAV particle may utilize or be based on a sequence, fragment or variant thereof, of the sequences in Table 1.

In one embodiment, the AAV particle may utilize or be based on a sequence, fragment or variant as described in Table 1.

TABLE 1
AAV Serotypes

| Serotype | SEQ ID NO | Reference Information |
|---|---|---|
| AAV1 | 25 | US20150159173 SEQ ID NO: 11, US20150315612 SEQ |
| | | ID NO: 202 |
| AAV1 | 26 | US20160017295 SEQ ID NO: 1, US20030138772 SEQ |
| | | ID NO: 64, US20150159173 SEQ ID NO: 27, |
| | | US20150315612 SEQ ID NO: 219, U.S. Pat. No. 7,198,951 SEQ ID |
| | | NO: 5 |
| AAV1 | 27 | US20030138772 SEQ ID NO: 6 |
| AAV1.3 | 28 | US20030138772 SEQ ID NO: 14 |
| AAV10 | 29 | US20030138772 SEQ ID NO: 117 |
| AAV10 | 30 | WO2015121501 SEQ ID NO: 9 |
| AAV10 | 31 | WO2015121501 SEQ ID NO: 8 |
| AAV11 | 32 | US20030138772 SEQ ID NO: 118 |
| AAV12 | 33 | US20030138772 SEQ ID NO: 119 |
| AAV2 | 34 | US20150159173 SEQ ID NO: 7, US20150315612 SEQ |
| | | ID NO: 211 |
| AAV2 | 35 | US20030138772 SEQ ID NO: 70, US20150159173 SEQ |
| | | ID NO: 23, US20150315612 SEQ ID NO: 221, |
| | | US20160017295 SEQ ID NO: 2, U.S. Pat. No. 6,156,303 SEQ ID |
| | | NO: 4, U.S. Pat. No. 7,198,951 SEQ ID NO: 4, WO2015121501 |
| | | SEQ ID NO: 1 |
| AAV2 | 36 | U.S. Pat. No. 6,156,303 SEQ ID NO: 8 |
| AAV2 | 37 | US20030138772 SEQ ID NO: 7 |
| AAV2 | 38 | U.S. Pat. No. 6,156,303 SEQ ID NO: 3 |
| AAV2.5T | 39 | U.S. Pat. No. 9,233,131 SEQ ID NO: 42 |
| AAV223.10 | 40 | US20030138772 SEQ ID NO: 75 |
| AAV223.2 | 41 | US20030138772 SEQ ID NO: 49 |
| AAV223.2 | 42 | US20030138772 SEQ ID NO: 76 |
| AAV223.4 | 43 | US20030138772 SEQ ID NO: 50 |
| AAV223.4 | 44 | US20030138772 SEQ ID NO: 73 |
| AAV223.5 | 45 | US20030138772 SEQ ID NO: 51 |
| AAV223.5 | 46 | US20030138772 SEQ ID NO: 74 |
| AAV223.6 | 47 | US20030138772 SEQ ID NO: 52 |
| AAV223.6 | 48 | US20030138772 SEQ ID NO: 78 |
| AAV223.7 | 49 | US20030138772 SEQ ID NO: 53 |
| AAV223.7 | 50 | US20030138772 SEQ ID NO: 77 |
| AAV29.3 | 51 | US20030138772 SEQ ID NO: 82 |
| AAV29.4 | 52 | US20030138772 SEQ ID NO: 12 |
| AAV29.5 | 53 | US20030138772 SEQ ID NO: 83 |
| AAV29.5 (AAVbb.2) | 54 | US20030138772 SEQ ID NO: 13 |
| AAV3 | 55 | US20150159173 SEQ ID NO: 12 |
| AAV3 | 56 | US20030138772 SEQ ID NO: 71, US20150159173 SEQ |
| | | ID NO: 28, US20160017295 SEQ ID NO: 3, |
| | | U.S. Pat. No. 7,198,951 SEQ ID NO: 6 |
| AAV3 | 57 | US20030138772 SEQ ID NO: 8 |
| AAV3.3b | 58 | US20030138772 SEQ ID NO: 72 |
| AAV3-3 | 59 | US20150315612 SEQ ID NO: 200 |
| AAV3-3 | 60 | US20150315612 SEQ ID NO: 217 |
| AAV3a | 61 | U.S. Pat. No. 6,156,303 SEQ ID NO: 5 |
| AAV3a | 62 | U.S. Pat. No. 6,156,303 SEQ ID NO: 9 |
| AAV3b | 63 | U.S. Pat. No. 6,156,303 SEQ ID NO: 6 |
| AAV3b | 64 | U.S. Pat. No. 6,156,303 SEQ ID NO: 10 |
| AAV3b | 65 | U.S. Pat. No. 6,156,303 SEQ ID NO: 1 |
| AAV4 | 66 | US20140348794 SEQ ID NO: 17 |
| AAV4 | 67 | US20140348794 SEQ ID NO: 5 |
| AAV4 | 68 | US20140348794 SEQ ID NO: 3 |
| AAV4 | 69 | US20140348794 SEQ ID NO: 14 |
| AAV4 | 70 | US20140348794 SEQ ID NO: 15 |
| AAV4 | 71 | US20140348794 SEQ ID NO: 19 |
| AAV4 | 72 | US20140348794 SEQ ID NO: 12 |
| AAV4 | 73 | US20140348794 SEQ ID NO: 13 |
| AAV4 | 74 | US20140348794 SEQ ID NO: 7 |
| AAV4 | 75 | US20140348794 SEQ ID NO: 8 |
| AAV4 | 76 | US20140348794 SEQ ID NO: 9 |
| AAV4 | 77 | US20140348794 SEQ ID NO: 2 |
| AAV4 | 78 | US20140348794 SEQ ID NO: 10 |
| AAV4 | 79 | US20140348794 SEQ ID NO: 11 |
| AAV4 | 80 | US20140348794 SEQ ID NO: 18 |
| AAV4 | 81 | US20030138772 SEQ ID NO: 63, US20160017295 SEQ |
| | | ID NO: 4, US20140348794 SEQ ID NO: 4 |
| AAV4 | 82 | US20140348794 SEQ ID NO: 16 |
| AAV4 | 83 | US20140348794 SEQ ID NO: 20 |
| AAV4 | 84 | US20140348794 SEQ ID NO: 6 |
| AAV4 | 85 | US20140348794 SEQ ID NO: 1 |
| AAV42.2 | 86 | US20030138772 SEQ ID NO: 9 |
| AAV42.2 | 87 | US20030138772 SEQ ID NO: 102 |
| AAV42.3b | 88 | US20030138772 SEQ ID NO: 36 |
| AAV42.3B | 89 | US20030138772 SEQ ID NO: 107 |
| AAV42.4 | 90 | US20030138772 SEQ ID NO: 33 |
| AAV42.4 | 91 | US20030138772 SEQ ID NO: 88 |
| AAV42.8 | 92 | US20030138772 SEQ ID NO: 27 |
| AAV42.8 | 93 | US20030138772 SEQ ID NO: 85 |
| AAV43.1 | 94 | US20030138772 SEQ ID NO: 39 |
| AAV43.1 | 95 | US20030138772 SEQ ID NO: 92 |
| AAV43.12 | 96 | US20030138772 SEQ ID NO: 41 |
| AAV43.12 | 97 | US20030138772 SEQ ID NO: 93 |
| AAV43.20 | 98 | US20030138772 SEQ ID NO: 42 |
| AAV43.20 | 99 | US20030138772 SEQ ID NO: 99 |
| AAV43.21 | 100 | US20030138772 SEQ ID NO: 43 |
| AAV43.21 | 101 | US20030138772 SEQ ID NO: 96 |
| AAV43.23 | 102 | US20030138772 SEQ ID NO: 44 |
| AAV43.23 | 103 | US20030138772 SEQ ID NO: 98 |
| AAV43.25 | 104 | US20030138772 SEQ ID NO: 45 |
| AAV43.25 | 105 | US20030138772 SEQ ID NO: 97 |
| AAV43.5 | 106 | US20030138772 SEQ ID NO: 40 |
| AAV43.5 | 107 | US20030138772 SEQ ID NO: 94 |
| AAV4-4 | 108 | US20150315612 SEQ ID NO: 201 |
| AAV4-4 | 109 | US20150315612 SEQ ID NO: 218 |
| AAV44.1 | 110 | US20030138772 SEQ ID NO: 46 |
| AAV44.1 | 111 | US20030138772 SEQ ID NO: 79 |
| AAV44.5 | 112 | US20030138772 SEQ ID NO: 47 |
| AAV44.5 | 113 | US20030138772 SEQ ID NO: 80 |
| AAV4407 | 114 | US20150315612 SEQ ID NO: 90 |
| AAV5 | 115 | U.S. Pat. No. 7,427,396 SEQ ID NO: 1 |
| AAV5 | 116 | US20030138772 SEQ ID NO: 114 |
| AAV5 | 117 | US20160017295 SEQ ID NO: 5, U.S. Pat. No. 7,427,396 SEQ ID |
| | | NO: 2, US20150315612 SEQ ID NO: 216 |
| AAV5 | 118 | US20150315612 SEQ ID NO: 199 |
| AAV6 | 119 | US20150159173 SEQ ID NO: 13 |
| AAV6 | 120 | US20030138772 SEQ ID NO: 65, US20150159173 SEQ |
| | | ID NO: 29, US20160017295 SEQ ID NO: 6, |
| | | U.S. Pat. No. 6,156,303 SEQ ID NO: 7 |
| AAV6 | 121 | U.S. Pat. No. 6,156,303 SEQ ID NO: 11 |
| AAV6 | 122 | U.S. Pat. No. 6,156,303 SEQ ID NO: 2 |
| AAV6 | 123 | US20150315612 SEQ ID NO: 203 |
| AAV6 | 124 | US20150315612 SEQ ID NO: 220 |
| AAV6.1 | 125 | US20150159173 |
| AAV6.12 | 126 | US20150159173 |
| AAV6.2 | 127 | US20150159173 |
| AAV7 | 128 | US20150159173 SEQ ID NO: 14 |
| AAV7 | 129 | US20150315612 SEQ ID NO: 183 |
| AAV7 | 130 | US20030138772 SEQ ID NO: 2, US20150159173 SEQ |
| | | ID NO: 30, US20150315612 SEQ ID NO: 181, |
| | | US20160017295 SEQ ID NO: 7 |
| AAV7 | 131 | US20030138772 SEQ ID NO: 3 |
| AAV7 | 132 | US20030138772 SEQ ID NO: 1, US20150315612 SEQ |
| | | ID NO: 180 |
| AAV7 | 133 | US20150315612 SEQ ID NO: 213 |
| AAV7 | 134 | US20150315612 SEQ ID NO: 222 |
| AAV8 | 135 | US20150159173 SEQ ID NO: 15 |
| AAV8 | 136 | US20150376240 SEQ ID NO: 7 |
| AAV8 | 137 | US20030138772 SEQ ID NO: 4, US20150315612 SEQ |
| | | ID NO: 182 |
| AAV8 | 138 | US20030138772 SEQ ID NO: 95, US20140359799 SEQ |
| | | ID NO: 1, US20150159173 SEQ ID NO: 31, |
| | | US20160017295 SEQ ID NO: 8, U.S. Pat. No. 7,198,951 SEQ ID |
| | | NO: 7, US20150315612 SEQ ID NO: 223 |
| AAV8 | 139 | US20150376240 SEQ ID NO: 8 |
| AAV8 | 140 | US20150315612 SEQ ID NO: 214 |
| AAV-8b | 141 | US20150376240 SEQ ID NO: 5 |
| AAV-8b | 142 | US20150376240 SEQ ID NO: 3 |
| AAV-8h | 143 | US20150376240 SEQ ID NO: 6 |
| AAV-8h | 144 | US20150376240 SEQ ID NO: 4 |
| AAV9 | 145 | US20030138772 SEQ ID NO: 5 |
| AAV9 | 146 | U.S. Pat. No. 7,198,951 SEQ ID NO: 1 |
| AAV9 | 147 | US20160017295 SEQ ID NO: 9 |
| AAV9 | 148 | US20030138772 SEQ ID NO: 100, U.S. Pat. No. 7,198,951 SEQ ID |
| | | NO: 2 |
| AAV9 | 149 | U.S. Pat. No. 7,198,951 SEQ ID NO: 3 |
| AAV9 (AAVhu.14) | 150 | US20150315612 SEQ ID NO: 3 |
| AAV9 (AAVhu.14) | 151 | US20150315612 SEQ ID NO: 123 |
| AAVA3.1 | 152 | US20030138772 SEQ ID NO: 120 |
| AAVA3.3 | 153 | US20030138772 SEQ ID NO: 57 |
| AAVA3.3 | 154 | US20030138772 SEQ ID NO: 66 |
| AAVA3.4 | 155 | US20030138772 SEQ ID NO: 54 |
| AAVA3.4 | 156 | US20030138772 SEQ ID NO: 68 |
| AAVA3.5 | 157 | US20030138772 SEQ ID NO: 55 |
| AAVA3.5 | 158 | US20030138772 SEQ ID NO: 69 |
| AAVA3.7 | 159 | US20030138772 SEQ ID NO: 56 |
| AAVA3.7 | 160 | US20030138772 SEQ ID NO: 67 |
| AAV29.3 (AAVbb.1) | 161 | US20030138772 SEQ ID NO: 11 |
| AAVC2 | 162 | US20030138772 SEQ ID NO: 61 |
| AAVCh.5 | 163 | US20150159173 SEQ ID NO: 46, US20150315612 SEQ |
| | | ID NO: 234 |
| AAVcy.2 (AAV13.3) | 164 | US20030138772 SEQ ID NO: 15 |
| AAV24.1 | 165 | US20030138772 SEQ ID NO: 101 |
| AAVcy.3 (AAV24.1) | 166 | US20030138772 SEQ ID NO: 16 |
| AAV27.3 | 167 | US20030138772 SEQ ID NO: 104 |
| AAVcy.4 (AAV27.3) | 168 | US20030138772 SEQ ID NO: 17 |
| AAVcy.5 | 169 | US20150315612 SEQ ID NO: 227 |
| AAV7.2 | 170 | US20030138772 SEQ ID NO: 103 |
| AAVcy.5 (AAV7.2) | 171 | US20030138772 SEQ ID NO: 18 |
| AAV16.3 | 172 | US20030138772 SEQ ID NO: 105 |
| AAVcy.6 (AAV16.3) | 173 | US20030138772 SEQ ID NO: 10 |
| AAVcy.5 | 174 | US20150159173 SEQ ID NO: 8 |
| AAVcy.5 | 175 | US20150159173 SEQ ID NO: 24 |
| AAVCy.5R1 | 176 | US20150159173 |
| AAVCy.5R2 | 177 | US20150159173 |
| AAVCy.5R3 | 178 | US20150159173 |
| AAVCy.5R4 | 179 | US20150159173 |
| AAVDJ | 180 | US20140359799 SEQ ID NO: 3, U.S. Pat. No. 7,588,772 SEQ ID |
| | | NO: 2 |
| AAVDJ | 181 | US20140359799 SEQ ID NO: 2, U.S. Pat. No. 7,588,772 SEQ ID |
| | | NO: 1 |
| AAVDJ-8 | 182 | U.S. Pat. No. 7,588,772; Grimm et al 2008 |
| AAVDJ-8 | 183 | U.S. Pat. No. 7,588,772; Grimm et al 2008 |
| AAVF5 | 184 | US20030138772 SEQ ID NO: 110 |
| AAVH2 | 185 | US20030138772 SEQ ID NO: 26 |
| AAVH6 | 186 | US20030138772 SEQ ID NO: 25 |
| AAVhE1.1 | 187 | U.S. Pat. No. 9,233,131 SEQ ID NO: 44 |
| AAVhEr1.14 | 188 | U.S. Pat. No. 9,233,131 SEQ ID NO: 46 |
| AAVhEr1.16 | 189 | U.S. Pat. No. 9,233,131 SEQ ID NO: 48 |
| AAVhEr1.18 | 190 | U.S. Pat. No. 9,233,131 SEQ ID NO: 49 |
| AAVhEr1.23 (AAVhEr2.29) | 191 | U.S. Pat. No. 9,233,131 SEQ ID NO: 53 |
| AAVhEr1.35 | 192 | U.S. Pat. No. 9,233,131 SEQ ID NO: 50 |
| AAVhEr1.36 | 193 | U.S. Pat. No. 9,233,131 SEQ ID NO: 52 |
| AAVhEr1.5 | 194 | U.S. Pat. No. 9,233,131 SEQ ID NO: 45 |
| AAVhEr1.7 | 195 | U.S. Pat. No. 9,233,131 SEQ ID NO: 51 |
| AAVhEr1.8 | 196 | U.S. Pat. No. 9,233,131 SEQ ID NO: 47 |
| AAVhEr2.16 | 197 | U.S. Pat. No. 9,233,131 SEQ ID NO: 55 |
| AAVhEr2.30 | 198 | U.S. Pat. No. 9,233,131 SEQ ID NO: 56 |
| AAVhEr2.31 | 199 | U.S. Pat. No. 9,233,131 SEQ ID NO: 58 |
| AAVhEr2.36 | 200 | U.S. Pat. No. 9,233,131 SEQ ID NO: 57 |
| AAVhEr2.4 | 201 | U.S. Pat. No. 9,233,131 SEQ ID NO: 54 |
| AAVhEr3.1 | 202 | U.S. Pat. No. 9,233,131 SEQ ID NO: 59 |
| AAVhu.1 | 203 | US20150315612 SEQ ID NO: 46 |
| AAVhu.1 | 204 | US20150315612 SEQ ID NO: 144 |
| AAVhu.10 (AAV16.8) | 205 | US20150315612 SEQ ID NO: 56 |
| AAVhu.10 (AAV16.8) | 206 | US20150315612 SEQ ID NO: 156 |
| AAVhu.11 (AAV16.12) | 207 | US20150315612 SEQ ID NO: 57 |
| AAVhu.11 (AAV16.12) | 208 | US20150315612 SEQ ID NO: 153 |
| AAVhu.12 | 209 | US20150315612 SEQ ID NO: 59 |
| AAVhu.12 | 210 | US20150315612 SEQ ID NO: 154 |
| AAVhu.13 | 211 | US20150159173 SEQ ID NO: 16, US20150315612 SEQ |
| | | ID NO: 71 |
| AAVhu.13 | 212 | US20150159173 SEQ ID NO: 32, US20150315612 SEQ |
| | | ID NO: 129 |
| AAVhu.136.1 | 213 | US20150315612 SEQ ID NO: 165 |
| AAVhu.140.1 | 214 | US20150315612 SEQ ID NO: 166 |
| AAVhu.140.2 | 215 | US20150315612 SEQ ID NO: 167 |
| AAVhu.145.6 | 216 | US20150315612 SEQ ID No: 178 |
| AAVhu.15 | 217 | US20150315612 SEQ ID NO: 147 |
| AAVhu.15 (AAV33.4) | 218 | US20150315612 SEQ ID NO: 50 |
| AAVhu.156.1 | 219 | US20150315612 SEQ ID No: 179 |
| AAVhu.16 | 220 | US20150315612 SEQ ID NO: 148 |
| AAVhu.16 (AAV33.8) | 221 | US20150315612 SEQ ID NO: 51 |
| AAVhu.17 | 222 | US20150315612 SEQ ID NO: 83 |
| AAVhu.17 (AAV33.12) | 223 | US20150315612 SEQ ID NO: 4 |
| AAVhu.172.1 | 224 | US20150315612 SEQ ID NO: 171 |
| AAVhu.172.2 | 225 | US20150315612 SEQ ID NO: 172 |
| AAVhu.173.4 | 226 | US20150315612 SEQ ID NO: 173 |
| AAVhu.173.8 | 227 | US20150315612 SEQ ID NO: 175 |
| AAVhu.18 | 228 | US20150315612 SEQ ID NO: 52 |
| AAVhu.18 | 229 | US20150315612 SEQ ID NO: 149 |
| AAVhu.19 | 230 | US20150315612 SEQ ID NO: 62 |
| AAVhu.19 | 231 | US20150315612 SEQ ID NO: 133 |
| AAVhu.2 | 232 | US20150315612 SEQ ID NO: 48 |
| AAVhu.2 | 233 | US20150315612 SEQ ID NO: 143 |
| AAVhu.20 | 234 | US20150315612 SEQ ID NO: 63 |
| AAVhu.20 | 235 | US20150315612 SEQ ID NO: 134 |
| AAVhu.21 | 236 | US20150315612 SEQ ID NO: 65 |
| AAVhu.21 | 237 | US20150315612 SEQ ID NO: 135 |
| AAVhu.22 | 238 | US20150315612 SEQ ID NO: 67 |
| AAVhu.22 | 239 | US20150315612 SEQ ID NO: 138 |
| AAVhu.23 | 240 | US20150315612 SEQ ID NO: 60 |
| AAVhu.23.2 | 241 | US20150315612 SEQ ID NO: 137 |
| AAVhu.24 | 242 | US20150315612 SEQ ID NO: 66 |
| AAVhu.24 | 243 | US20150315612 SEQ ID NO: 136 |
| AAVhu.25 | 244 | US20150315612 SEQ ID NO: 49 |
| AAVhu.25 | 245 | US20150315612 SEQ ID NO: 146 |
| AAVhu.26 | 246 | US20150159173 SEQ ID NO: 17, US20150315612 SEQ |
| | | ID NO: 61 |
| AAVhu.26 | 247 | US20150159173 SEQ ID NO: 33, US20150315612 SEQ |
| | | ID NO: 139 |
| AAVhu.27 | 248 | US20150315612 SEQ ID NO: 64 |
| AAVhu.27 | 249 | US20150315612 SEQ ID NO: 140 |
| AAVhu.28 | 250 | US20150315612 SEQ ID NO: 68 |
| AAVhu.28 | 251 | US20150315612 SEQ ID NO: 130 |
| AAVhu.29 | 252 | US20150315612 SEQ ID NO: 69 |
| AAVhu.29 | 253 | US20150159173 SEQ ID NO: 42, US20150315612 SEQ |
| | | ID NO: 132 |
| AAVhu.29 | 254 | US20150315612 SEQ ID NO: 225 |
| AAVhu.29R | 255 | US20150159173 |
| AAVhu.3 | 256 | US20150315612 SEQ ID NO: 44 |
| AAVhu.3 | 257 | US20150315612 SEQ ID NO: 145 |
| AAVhu.30 | 258 | US20150315612 SEQ ID NO: 70 |
| AAVhu.30 | 259 | US20150315612 SEQ ID NO: 131 |
| AAVhu.31 | 260 | US20150315612 SEQ ID NO: 1 |
| AAVhu.31 | 261 | US20150315612 SEQ ID NO: 121 |
| AAVhu.32 | 262 | US20150315612 SEQ ID NO: 2 |
| AAVhu.32 | 263 | US20150315612 SEQ ID NO: 122 |
| AAVhu.33 | 264 | US20150315612 SEQ ID NO: 75 |
| AAVhu.33 | 265 | US20150315612 SEQ ID NO: 124 |
| AAVhu.34 | 266 | US20150315612 SEQ ID NO: 72 |
| AAVhu.34 | 267 | US20150315612 SEQ ID NO: 125 |
| AAVhu.35 | 268 | US20150315612 SEQ ID NO: 73 |
| AAVhu.35 | 269 | US20150315612 SEQ ID NO: 164 |
| AAVhu.36 | 270 | US20150315612 SEQ ID NO: 74 |
| AAVhu.36 | 271 | US20150315612 SEQ ID NO: 126 |
| AAVhu.37 | 272 | US20150159173 SEQ ID NO: 34, US20150315612 SEQ |
| | | ID NO: 88 |
| AAVhu.37 (AAV106.1) | 273 | US20150315612 SEQ ID NO: 10, US20150159173 SEQ |
| | | ID NO: 18 |
| AAVhu.38 | 274 | US20150315612 SEQ ID NO: 161 |
| AAVhu.39 | 275 | US20150315612 SEQ ID NO: 102 |
| AAVhu.39 (AAVLG-9) | 276 | US20150315612 SEQ ID NO: 24 |
| AAVhu.4 | 277 | US20150315612 SEQ ID NO: 47 |
| AAVhu.4 | 278 | US20150315612 SEQ ID NO: 141 |
| AAVhu.40 | 279 | US20150315612 SEQ ID NO: 87 |
| AAVhu.40 (AAV114.3) | 280 | US20150315612 SEQ ID No: 11 |
| AAVhu.41 | 281 | US20150315612 SEQ ID NO: 91 |
| AAVhu.41 (AAV127.2) | 282 | US20150315612 SEQ ID NO: 6 |
| AAVhu.42 | 283 | US20150315612 SEQ ID NO: 85 |
| AAVhu.42 (AAV127.5) | 284 | US20150315612 SEQ ID NO: 8 |
| AAVhu.43 | 285 | US20150315612 SEQ ID NO: 160 |
| AAVhu.43 | 286 | US20150315612 SEQ ID NO: 236 |
| AAVhu.43 (AAV128.1) | 287 | US20150315612 SEQ ID NO: 80 |
| AAVhu.44 | 288 | US20150159173 SEQ ID NO: 45, US20150315612 SEQ |
| | | ID NO: 158 |
| AAVhu.44 (AAV128.3) | 289 | US20150315612 SEQ ID NO: 81 |
| AAVhu.44R1 | 290 | US20150159173 |
| AAVhu.44R2 | 291 | US20150159173 |
| AAVhu.44R3 | 292 | US20150159173 |
| AAVhu.45 | 293 | US20150315612 SEQ ID NO: 76 |
| AAVhu.45 | 294 | US20150315612 SEQ ID NO: 127 |
| AAVhu.46 | 295 | US20150315612 SEQ ID NO: 82 |
| AAVhu.46 | 296 | US20150315612 SEQ ID NO: 159 |
| AAVhu.46 | 297 | US20150315612 SEQ ID NO: 224 |
| AAVhu.47 | 298 | US20150315612 SEQ ID NO: 77 |
| AAVhu.47 | 299 | US20150315612 SEQ ID NO: 128 |
| AAVhu.48 | 300 | US20150159173 SEQ ID NO: 38 |
| AAVhu.48 | 301 | US20150315612 SEQ ID NO: 157 |
| AAVhu.48 (AAV130.4) | 302 | US20150315612 SEQ ID NO: 78 |
| AAVhu.48R1 | 303 | US20150159173 |
| AAVhu.48R2 | 304 | US20150159173 |
| AAVhu.48R3 | 305 | US20150159173 |
| AAVhu.49 | 306 | US20150315612 SEQ ID NO: 209 |
| AAVhu.49 | 307 | US20150315612 SEQ ID NO: 189 |
| AAVhu.5 | 308 | US20150315612 SEQ ID NO: 45 |
| AAVhu.5 | 309 | US20150315612 SEQ ID NO: 142 |
| AAVhu.51 | 310 | US20150315612 SEQ ID NO: 208 |
| AAVhu.51 | 311 | US20150315612 SEQ ID NO: 190 |
| AAVhu.52 | 312 | US20150315612 SEQ ID NO: 210 |
| AAVhu.52 | 313 | US20150315612 SEQ ID NO: 191 |
| AAVhu.53 | 314 | US20150159173 SEQ ID NO: 19 |
| AAVhu.53 | 315 | US20150159173 SEQ ID NO: 35 |
| AAVhu.53 (AAV145.1) | 316 | US20150315612 SEQ ID NO: 176 |
| AAVhu.54 | 317 | US20150315612 SEQ ID NO: 188 |
| AAVhu.54 (AAV145.5) | 318 | US20150315612 SEQ ID No: 177 |
| AAVhu.55 | 319 | US20150315612 SEQ ID NO: 187 |
| AAVhu.56 | 320 | US20150315612 SEQ ID NO: 205 |
| AAVhu.56 (AAV145.6) | 321 | US20150315612 SEQ ID NO: 168 |
| AAVhu.56 (AAV145.6) | 322 | US20150315612 SEQ ID NO: 192 |
| AAVhu.57 | 323 | US20150315612 SEQ ID NO: 206 |
| AAVhu.57 | 324 | US20150315612 SEQ ID NO: 169 |
| AAVhu.57 | 325 | US20150315612 SEQ ID NO: 193 |
| AAVhu.58 | 326 | US20150315612 SEQ ID NO: 207 |
| AAVhu.58 | 327 | US20150315612 SEQ ID NO: 194 |
| AAVhu.6 (AAV3.1) | 328 | US20150315612 SEQ ID NO: 5 |
| AAVhu.6 (AAV3.1) | 329 | US20150315612 SEQ ID NO: 84 |
| AAVhu.60 | 330 | US20150315612 SEQ ID NO: 184 |
| AAVhu.60 (AAV161.10) | 331 | US20150315612 SEQ ID NO: 170 |
| AAVhu.61 | 332 | US20150315612 SEQ ID NO: 185 |
| AAVhu.61 (AAV161.6) | 333 | US20150315612 SEQ ID NO: 174 |
| AAVhu.63 | 334 | US20150315612 SEQ ID NO: 204 |
| AAVhu.63 | 335 | US20150315612 SEQ ID NO: 195 |
| AAVhu.64 | 336 | US20150315612 SEQ ID NO: 212 |
| AAVhu.64 | 337 | US20150315612 SEQ ID NO: 196 |
| AAVhu.66 | 338 | US20150315612 SEQ ID NO: 197 |
| AAVhu.67 | 339 | US20150315612 SEQ ID NO: 215 |
| AAVhu.67 | 340 | US20150315612 SEQ ID NO: 198 |
| AAVhu.7 | 341 | US20150315612 SEQ ID NO: 226 |
| AAVhu.7 | 342 | US20150315612 SEQ ID NO: 150 |
| AAVhu.7 (AAV7.3) | 343 | US20150315612 SEQ ID NO: 55 |
| AAVhu.71 | 344 | US20150315612 SEQ ID NO: 79 |
| AAVhu.8 | 345 | US20150315612 SEQ ID NO: 53 |
| AAVhu.8 | 346 | US20150315612 SEQ ID NO: 12 |
| AAVhu.8 | 347 | US20150315612 SEQ ID NO: 151 |
| AAVhu.9 (AAV3.1) | 348 | US20150315612 SEQ ID NO: 58 |
| AAVhu.9 (AAV3.1) | 349 | US20150315612 SEQ ID NO: 155 |
| AAV-LK01 | 350 | US20150376607 SEQ ID NO: 2 |
| AAV-LK01 | 351 | US20150376607 SEQ ID NO: 29 |
| AAV-LK02 | 352 | US20150376607 SEQ ID NO: 3 |
| AAV-LK02 | 353 | US20150376607 SEQ ID NO: 30 |
| AAV-LK03 | 354 | US20150376607 SEQ ID NO: 4 |
| AAV-LK03 | 355 | WO2015121501 SEQ ID NO: 12, US20150376607 SEQ |
| | | ID NO: 31 |
| AAV-LK04 | 356 | US20150376607 SEQ ID NO: 5 |
| AAV-LK04 | 357 | US20150376607 SEQ ID NO: 32 |
| AAV-LK05 | 358 | US20150376607 SEQ ID NO: 6 |
| AAV-LK05 | 359 | US20150376607 SEQ ID NO: 33 |
| AAV-LK06 | 360 | US20150376607 SEQ ID NO: 7 |
| AAV-LK06 | 361 | US20150376607 SEQ ID NO: 34 |
| AAV-LK07 | 362 | US20150376607 SEQ ID NO: 8 |
| AAV-LK07 | 363 | US20150376607 SEQ ID NO: 35 |
| AAV-LK08 | 364 | US20150376607 SEQ ID NO: 9 |
| AAV-LK08 | 365 | US20150376607 SEQ ID NO: 36 |
| AAV-LK09 | 366 | US20150376607 SEQ ID NO: 10 |
| AAV-LK09 | 367 | US20150376607 SEQ ID NO: 37 |
| AAV-LK10 | 368 | US20150376607 SEQ ID NO: 11 |
| AAV-LK10 | 369 | US20150376607 SEQ ID NO: 38 |
| AAV-LK11 | 370 | US20150376607 SEQ ID NO: 12 |
| AAV-LK11 | 371 | US20150376607 SEQ ID NO: 39 |
| AAV-LK12 | 372 | US20150376607 SEQ ID NO: 13 |
| AAV-LK12 | 373 | US20150376607 SEQ ID NO: 40 |
| AAV-LK13 | 374 | US20150376607 SEQ ID NO: 14 |
| AAV-LK13 | 375 | US20150376607 SEQ ID NO: 41 |
| AAV-LK14 | 376 | US20150376607 SEQ ID NO: 15 |
| AAV-LK14 | 377 | US20150376607 SEQ ID NO: 42 |
| AAV-LK15 | 378 | US20150376607 SEQ ID NO: 16 |
| AAV-LK15 | 379 | US20150376607 SEQ ID NO: 43 |
| AAV-LK16 | 380 | US20150376607 SEQ ID NO: 17 |
| AAV-LK16 | 381 | US20150376607 SEQ ID NO: 44 |
| AAV-LK17 | 382 | US20150376607 SEQ ID NO: 18 |
| AAV-LK17 | 383 | US20150376607 SEQ ID NO: 45 |
| AAV-LK18 | 384 | US20150376607 SEQ ID NO: 19 |
| AAV-LK18 | 385 | US20150376607 SEQ ID NO: 46 |
| AAV-LK19 | 386 | US20150376607 SEQ ID NO: 20 |
| AAV-LK19 | 387 | US20150376607 SEQ ID NO: 47 |
| AAV-PAEC | 388 | US20150376607 SEQ ID NO: 1 |
| AAV-PAEC | 389 | US20150376607 SEQ ID NO: 48 |
| AAV-PAEC11 | 390 | US20150376607 SEQ ID NO: 26 |
| AAV-PAEC11 | 391 | US20150376607 SEQ ID NO: 54 |
| AAV-PAEC12 | 392 | US20150376607 SEQ ID NO: 27 |
| AAV-PAEC12 | 393 | US20150376607 SEQ ID NO: 51 |
| AAV-PAEC13 | 394 | US20150376607 SEQ ID NO: 28 |
| AAV-PAEC13 | 395 | US20150376607 SEQ ID NO: 49 |
| AAV-PAEC2 | 396 | US20150376607 SEQ ID NO: 21 |
| AAV-PAEC2 | 397 | US20150376607 SEQ ID NO: 56 |
| AAV-PAEC4 | 398 | US20150376607 SEQ ID NO: 22 |
| AAV-PAEC4 | 399 | US20150376607 SEQ ID NO: 55 |
| AAV-PAEC6 | 400 | US20150376607 SEQ ID NO: 23 |
| AAV-PAEC6 | 401 | US20150376607 SEQ ID NO: 52 |
| AAV-PAEC7 | 402 | US20150376607 SEQ ID NO: 24 |
| AAV-PAEC7 | 403 | US20150376607 SEQ ID NO: 53 |
| AAV-PAEC8 | 404 | US20150376607 SEQ ID NO: 25 |
| AAV-PAEC8 | 405 | US20150376607 SEQ ID NO: 50 |
| AAVpi.1 | 406 | US20150315612 SEQ ID NO: 28 |
| AAVpi.1 | 407 | US20150315612 SEQ ID NO: 93 |
| AAVpi.2 | 408 | US20150315612 SEQ ID NO: 30 |
| AAVpi.2 | 409 | US20150315612 SEQ ID NO: 95 |
| AAVpi.3 | 410 | US20150315612 SEQ ID NO: 29 |
| AAVpi.3 | 411 | US20150315612 SEQ ID NO: 94 |
| AAVrh.10 | 412 | US20150159173 SEQ ID NO: 9 |
| AAVrh.10 | 413 | US20150159173 SEQ ID NO: 25 |
| AAV44.2 | 414 | US20030138772 SEQ ID NO: 59 |
| AAVrh.10 (AAV44.2) | 415 | US20030138772 SEQ ID NO: 81 |
| AAV42.1B | 416 | US20030138772 SEQ ID NO: 90 |
| AAVrh.12 (AAV42.1b) | 417 | US20030138772 SEQ ID NO: 30 |
| AAVrh.13 | 418 | US20150159173 SEQ ID NO: 10 |
| AAVrh.13 | 419 | US20150159173 SEQ ID NO: 26 |
| AAVrh.13 | 420 | US20150315612 SEQ ID NO: 228 |
| AAVrh.13R | 421 | US20150159173 |
| AAV42.3A | 422 | US20030138772 SEQ ID NO: 87 |
| AAVrh.14 (AAV42.3a) | 423 | US20030138772 SEQ ID NO: 32 |
| AAV42.5A | 424 | US20030138772 SEQ ID NO: 89 |
| AAVrh.17 (AAV42.5a) | 425 | US20030138772 SEQ ID NO: 34 |
| AAV42.5B | 426 | US20030138772 SEQ ID NO: 91 |
| AAVrh.18 (AAV42.5b) | 427 | US20030138772 SEQ ID NO: 29 |
| AAV42.6B | 428 | US20030138772 SEQ ID NO: 112 |
| AAVrh.19 (AAV42.6b) | 429 | US20030138772 SEQ ID NO: 38 |
| AAVrh.2 | 430 | US20150159173 SEQ ID NO: 39 |
| AAVrh.2 | 431 | US20150315612 SEQ ID NO: 231 |
| AAVrh.20 | 432 | US20150159173 SEQ ID NO: 1 |
| AAV42.10 | 433 | US20030138772 SEQ ID NO: 106 |
| AAVrh.21 (AAV42.10) | 434 | US20030138772 SEQ ID NO: 35 |
| AAV42.11 | 435 | US20030138772 SEQ ID NO: 108 |
| AAVrh.22 (AAV42.11) | 436 | US20030138772 SEQ ID NO: 37 |
| AAV42.12 | 437 | US20030138772 SEQ ID NO: 113 |
| AAVrh.23 (AAV42.12) | 438 | US20030138772 SEQ ID NO: 58 |
| AAV42.13 | 439 | US20030138772 SEQ ID NO: 86 |
| AAVrh.24 (AAV42.13) | 440 | US20030138772 SEQ ID NO: 31 |
| AAV42.15 | 441 | US20030138772 SEQ ID NO: 84 |
| AAVrh.25 (AAV42.15) | 442 | US20030138772 SEQ ID NO: 28 |
| AAVrh.2R | 443 | US20150159173 |
| AAVrh.31 (AAV223.1) | 444 | US20030138772 SEQ ID NO: 48 |
| AAVC1 | 445 | US20030138772 SEQ ID NO: 60 |
| AAVrh.32 (AAVC1) | 446 | US20030138772 SEQ ID NO: 19 |
| AAVrh.32/33 | 447 | US20150159173 SEQ ID NO: 2 |
| AAVrh.33 (AAVC3) | 448 | US20030138772 SEQ ID NO: 20 |
| AAVC5 | 449 | US20030138772 SEQ ID NO: 62 |
| AAVrh.34 (AAVC5) | 450 | US20030138772 SEQ ID NO: 21 |
| AAVF1 | 451 | US20030138772 SEQ ID NO: 109 |
| AAVrh.35 (AAVF1) | 452 | US20030138772 SEQ ID NO: 22 |
| AAVF3 | 453 | US20030138772 SEQ ID NO: 111 |
| AAVrh.36 (AAVF3) | 454 | US20030138772 SEQ ID NO: 23 |
| AAVrh.37 | 455 | US20030138772 SEQ ID NO: 24 |
| AAVrh.37 | 456 | US20150159173 SEQ ID NO: 40 |
| AAVrh.37 | 457 | US20150315612 SEQ ID NO: 229 |
| AAVrh.37R2 | 458 | US20150159173 |
| AAVrh.38 (AAVLG-4) | 459 | US20150315612 SEQ ID NO: 7 |
| AAVrh.38 (AAVLG-4) | 460 | US20150315612 SEQ ID NO: 86 |
| AAVrh.39 | 461 | US20150159173 SEQ ID NO: 20, US20150315612 SEQ |
| | | ID NO: 13 |
| AAVrh.39 | 462 | US20150159173 SEQ ID NO: 3, US20150159173 SEQ |
| | | ID NO: 36, US20150315612 SEQ ID NO: 89 |
| AAVrh.40 | 463 | US20150315612 SEQ ID NO: 92 |
| AAVrh.40 (AAVLG-10) | 464 | US20150315612 SEQ ID No: 14 |
| AAVrh.43 (AAVN721-8) | 465 | US20150315612 SEQ ID NO: 43, US20150159173 SEQ |
| | | ID NO: 21 |
| AAVrh.43 (AAVN721-8) | 466 | US20150315612 SEQ ID NO: 163, US20150159173 |
| | | SEQ ID NO: 37 |
| AAVrh.44 | 467 | US20150315612 SEQ ID NO: 34 |
| AAVrh.44 | 468 | US20150315612 SEQ ID NO: 111 |
| AAVrh.45 | 469 | US20150315612 SEQ ID NO: 41 |
| AAVrh.45 | 470 | US20150315612 SEQ ID NO: 109 |
| AAVrh.46 | 471 | US20150159173 SEQ ID NO: 22, US20150315612 SEQ |
| | | ID NO: 19 |
| AAVrh.46 | 472 | US20150159173 SEQ ID NO: 4, US20150315612 SEQ |
| | | ID NO: 101 |
| AAVrh.47 | 473 | US20150315612 SEQ ID NO: 38 |
| AAVrh.47 | 474 | US20150315612 SEQ ID NO: 118 |
| AAVrh.48 | 475 | US20150159173 SEQ ID NO: 44, US20150315612 SEQ |
| | | ID NO: 115 |
| AAVrh.48.1 | 476 | US20150159173 |
| AAVrh.48.1.2 | 477 | US20150159173 |
| AAVrh.48.2 | 478 | US20150159173 |
| AAVrh.48 (AAV1-7) | 479 | US20150315612 SEQ ID NO: 32 |
| AAVrh.49 (AAV1-8) | 480 | US20150315612 SEQ ID NO: 25 |
| AAVrh.49 (AAV1-8) | 481 | US20150315612 SEQ ID NO: 103 |
| AAVrh.50 (AAV2-4) | 482 | US20150315612 SEQ ID NO: 23 |
| AAVrh.50 (AAV2-4) | 483 | US20150315612 SEQ ID NO: 108 |
| AAVrh.51 (AAV2-5) | 484 | US20150315612 SEQ ID No: 22 |
| AAVrh.51 (AAV2-5) | 485 | US20150315612 SEQ ID NO: 104 |
| AAVrh.52 (AAV3-9) | 486 | US20150315612 SEQ ID NO: 18 |
| AAVrh.52 (AAV3-9) | 487 | US20150315612 SEQ ID NO: 96 |
| AAVrh.53 | 488 | US20150315612 SEQ ID NO: 97 |
| AAVrh.53 (AAV3-11) | 489 | US20150315612 SEQ ID NO: 17 |
| AAVrh.53 (AAV3-11) | 490 | US20150315612 SEQ ID NO: 186 |
| AAVrh.54 | 491 | US20150315612 SEQ ID NO: 40 |
| AAVrh.54 | 492 | US20150159173 SEQ ID NO: 49, US20150315612 SEQ |
| | | ID NO: 116 |
| AAVrh.55 | 493 | US20150315612 SEQ ID NO: 37 |
| AAVrh.55 (AAV4-19) | 494 | US20150315612 SEQ ID NO: 117 |
| AAVrh.56 | 495 | US20150315612 SEQ ID NO: 54 |
| AAVrh.56 | 496 | US20150315612 SEQ ID NO: 152 |
| AAVrh.57 | 497 | US20150315612 SEQ ID NO: 26 |
| AAVrh.57 | 498 | US20150315612 SEQ ID NO: 105 |
| AAVrh.58 | 499 | US20150315612 SEQ ID NO: 27 |
| AAVrh.58 | 500 | US20150159173 SEQ ID NO: 48, US20150315612 SEQ |
| | | ID NO: 106 |
| AAVrh.58 | 501 | US20150315612 SEQ ID NO: 232 |
| AAVrh.59 | 502 | US20150315612 SEQ ID NO: 42 |
| AAVrh.59 | 503 | US20150315612 SEQ ID NO: 110 |
| AAVrh.60 | 504 | US20150315612 SEQ ID NO: 31 |
| AAVrh.60 | 505 | US20150315612 SEQ ID NO: 120 |
| AAVrh.61 | 506 | US20150315612 SEQ ID NO: 107 |
| AAVrh.61 (AAV2-3) | 507 | US20150315612 SEQ ID NO: 21 |
| AAVrh.62 (AAV2-15) | 508 | US20150315612 SEQ ID No: 33 |
| AAVrh.62 (AAV2-15) | 509 | US20150315612 SEQ ID NO: 114 |
| AAVrh.64 | 510 | US20150315612 SEQ ID No: 15 |
| AAVrh.64 | 511 | US20150159173 SEQ ID NO: 43, US20150315612 SEQ |
| | | ID NO: 99 |
| AAVrh.64 | 512 | US20150315612 SEQ ID NO: 233 |
| AAVRh.64R1 | 513 | US20150159173 |
| AAVRh.64R2 | 514 | US20150159173 |
| AAVrh.65 | 515 | US20150315612 SEQ ID NO: 35 |
| AAVrh.65 | 516 | US20150315612 SEQ ID NO: 112 |
| AAVrh.67 | 517 | US20150315612 SEQ ID NO: 36 |
| AAVrh.67 | 518 | US20150315612 SEQ ID NO: 230 |
| AAVrh.67 | 519 | US20150159173 SEQ ID NO: 47, US20150315612 SEQ |
| | | ID NO: 113 |
| AAVrh.68 | 520 | US20150315612 SEQ ID NO: 16 |
| AAVrh.68 | 521 | US20150315612 SEQ ID NO: 100 |
| AAVrh.69 | 522 | US20150315612 SEQ ID NO: 39 |
| AAVrh.69 | 523 | US20150315612 SEQ ID NO: 119 |
| AAVrh.70 | 524 | US20150315612 SEQ ID NO: 20 |
| AAVrh.70 | 525 | US20150315612 SEQ ID NO: 98 |
| AAVrh.71 | 526 | US20150315612 SEQ ID NO: 162 |
| AAVrh.72 | 527 | US20150315612 SEQ ID NO: 9 |
| AAVrh.73 | 528 | US20150159173 SEQ ID NO: 5 |
| AAVrh.74 | 529 | US20150159173 SEQ ID NO: 6 |
| AAVrh.8 | 530 | US20150159173 SEQ ID NO: 41 |
| AAVrh.8 | 531 | US20150315612 SEQ ID NO: 235 |
| AAVrh.8R | 532 | US20150159173, WO2015168666 SEQ ID NO: 9 |
| AAVrh.8R A586R mutant | 533 | WO2015168666 SEQ ID NO: 10 |
| AAVrh.8R R533A mutant | 534 | WO2015168666 SEQ ID NO: 11 |
| BAAV (bovine AAV) | 535 | U.S. Pat. No. 9,193,769 SEQ ID NO: 8 |
| BAAV (bovine AAV) | 536 | U.S. Pat. No. 9,193,769 SEQ ID NO: 10 |
| BAAV (bovine AAV) | 537 | U.S. Pat. No. 9,193,769 SEQ ID NO: 4 |
| BAAV (bovine AAV) | 538 | U.S. Pat. No. 9,193,769 SEQ ID NO: 2 |
| BAAV (bovine AAV) | 539 | U.S. Pat. No. 9,193,769 SEQ ID NO: 6 |
| BAAV (bovine AAV) | 540 | U.S. Pat. No. 9,193,769 SEQ ID NO: 1 |
| BAAV (bovine AAV) | 541 | U.S. Pat. No. 9,193,769 SEQ ID NO: 5 |
| BAAV (bovine AAV) | 542 | U.S. Pat. No. 9,193,769 SEQ ID NO: 3 |
| BAAV (bovine AAV) | 543 | U.S. Pat. No. 9,193,769 SEQ ID NO: 11 |
| BAAV (bovine AAV) | 544 | U.S. Pat. No. 7,427,396 SEQ ID NO: 5 |
| BAAV (bovine AAV) | 545 | U.S. Pat. No. 7,427,396 SEQ ID NO: 6 |
| BAAV (bovine AAV) | 546 | U.S. Pat. No. 9,193,769 SEQ ID NO: 7 |
| BAAV (bovine AAV) | 547 | U.S. Pat. No. 9,193,769 SEQ ID NO: 9 |
| BNP61 AAV | 548 | US20150238550 SEQ ID NO: 1 |
| BNP61 AAV | 549 | US20150238550 SEQ ID NO: 2 |
| BNP62 AAV | 550 | US20150238550 SEQ ID NO: 3 |
| BNP63 AAV | 551 | US20150238550 SEQ ID NO: 4 |
| caprine AAV | 552 | U.S. Pat. No. 7,427,396 SEQ ID NO: 3 |
| caprine AAV | 553 | U.S. Pat. No. 7,427,396 SEQ ID NO: 4 |
| true type AAV (ttAAV) | 554 | WO2015121501 SEQ ID NO: 2 |
| AAAV (Avian AAV) | 555 | U.S. Pat. No. 9,238,800 SEQ ID NO: 12 |
| AAAV (Avian AAV) | 556 | U.S. Pat. No. 9,238,800 SEQ ID NO: 2 |
| AAAV (Avian AAV) | 557 | U.S. Pat. No. 9,238,800 SEQ ID NO: 6 |
| AAAV (Avian AAV) | 558 | U.S. Pat. No. 9,238,800 SEQ ID NO: 4 |
| AAAV (Avian AAV) | 559 | U.S. Pat. No. 9,238,800 SEQ ID NO: 8 |
| AAAV (Avian AAV) | 560 | U.S. Pat. No. 9,238,800 SEQ ID NO: 14 |
| AAAV (Avian AAV) | 561 | U.S. Pat. No. 9,238,800 SEQ ID NO: 10 |
| AAAV (Avian AAV) | 562 | U.S. Pat. No. 9,238,800 SEQ ID NO: 15 |
| AAAV (Avian AAV) | 563 | U.S. Pat. No. 9,238,800 SEQ ID NO: 5 |
| AAAV (Avian AAV) | 564 | U.S. Pat. No. 9,238,800 SEQ ID NO: 9 |
| AAAV (Avian AAV) | 565 | U.S. Pat. No. 9,238,800 SEQ ID NO: 3 |
| AAAV (Avian AAV) | 566 | U.S. Pat. No. 9,238,800 SEQ ID NO: 7 |
| AAAV (Avian AAV) | 567 | U.S. Pat. No. 9,238,800 SEQ ID NO: 11 |
| AAAV (Avian AAV) | 568 | U.S. Pat. No. 9,238,800 SEQ ID NO: 13 |
| AAAV (Avian AAV) | 569 | U.S. Pat. No. 9,238,800 SEQ ID NO: 1 |
| AAV Shuffle 100-1 | 570 | US20160017295 SEQ ID NO: 23 |
| AAV Shuffle 100-1 | 571 | US20160017295 SEQ ID NO: 11 |
| AAV Shuffle 100-2 | 572 | US20160017295 SEQ ID NO: 37 |
| AAV Shuffle 100-2 | 573 | US20160017295 SEQ ID NO: 29 |
| AAV Shuffle 100-3 | 574 | US20160017295 SEQ ID NO: 24 |
| AAV Shuffle 100-3 | 575 | US20160017295 SEQ ID NO: 12 |
| AAV Shuffle 100-7 | 576 | US20160017295 SEQ ID NO: 25 |
| AAV Shuffle 100-7 | 577 | US20160017295 SEQ ID NO: 13 |
| AAV Shuffle 10-2 | 578 | US20160017295 SEQ ID NO: 34 |
| AAV Shuffle 10-2 | 579 | US20160017295 SEQ ID NO: 26 |
| AAV Shuffle 10-6 | 580 | US20160017295 SEQ ID NO: 35 |
| AAV Shuffle 10-6 | 581 | US20160017295 SEQ ID NO: 27 |
| AAV Shuffle 10-8 | 582 | US20160017295 SEQ ID NO: 36 |
| AAV Shuffle 10-8 | 583 | US20160017295 SEQ ID NO: 28 |
| AAV SM 100-10 | 584 | US20160017295 SEQ ID NO: 41 |
| AAV SM 100-10 | 585 | US20160017295 SEQ ID NO: 33 |
| AAV SM 100-3 | 586 | US20160017295 SEQ ID NO: 40 |
| AAV SM 100-3 | 587 | US20160017295 SEQ ID NO: 32 |
| AAV SM 10-1 | 588 | US20160017295 SEQ ID NO: 38 |
| AAV SM 10-1 | 589 | US20160017295 SEQ ID NO: 30 |
| AAV SM 10-2 | 590 | US20160017295 SEQ ID NO: 10 |
| AAV SM 10-2 | 591 | US20160017295 SEQ ID NO: 22 |
| AAV SM 10-8 | 592 | US20160017295 SEQ ID NO: 39 |
| AAV SM 10-8 | 593 | US20160017295 SEQ ID NO: 31 |
| AAV CBr-7.1 | 594 | WO2016065001 SEQ ID NO: 4 |
| AAV CBr-7.1 | 595 | WO2016065001 SEQ ID NO: 54 |
| AAV CBr-7.10 | 596 | WO2016065001 SEQ ID NO: 11 |
| AAV CBr-7.10 | 597 | WO2016065001 SEQ ID NO: 61 |
| AAV CBr-7.2 | 598 | WO2016065001 SEQ ID NO: 5 |
| AAV CBr-7.2 | 599 | WO2016065001 SEQ ID NO: 55 |
| AAV CBr-7.3 | 600 | WO2016065001 SEQ ID NO: 6 |
| AAV CBr-7.3 | 601 | WO2016065001 SEQ ID NO: 56 |
| AAV CBr-7.4 | 602 | WO2016065001 SEQ ID NO: 7 |
| AAV CBr-7.4 | 603 | WO2016065001 SEQ ID NO: 57 |
| AAV CBr-7.5 | 604 | WO2016065001 SEQ ID NO: 8 |
| AAV CBr-7.5 | 605 | WO2016065001 SEQ ID NO: 58 |
| AAV CBr-7.7 | 606 | WO2016065001 SEQ ID NO: 9 |
| AAV CBr-7.7 | 607 | WO2016065001 SEQ ID NO: 59 |
| AAV CBr-7.8 | 608 | WO2016065001 SEQ ID NO: 10 |
| AAV CBr-7.8 | 609 | WO2016065001 SEQ ID NO: 60 |
| AAV CBr-B7.3 | 610 | WO2016065001 SEQ ID NO: 46 |
| AAV CBr-B7.3 | 611 | WO2016065001 SEQ ID NO: 96 |
| AAV CBr-B7.4 | 612 | WO2016065001 SEQ ID NO: 47 |
| AAV CBr-B7.4 | 613 | WO2016065001 SEQ ID NO: 97 |
| AAV CBr-E1 (AAV CLv- | 614 | U.S. Pat. No. 8,734,809 SEQ ID NO: 13 |
| E1) | | |
| AAV CBr-E1 (AAV CLv- | 615 | U.S. Pat. No. 8,734,809 SEQ ID NO: 87 |
| E1) | | |
| AAV CBr-E2 | 616 | U.S. Pat. No. 8,734,809 SEQ ID NO: 14 |
| AAV CBr-E2 | 617 | U.S. Pat. No. 8,734,809 SEQ ID NO: 88 |
| AAV CBr-E3 | 618 | U.S. Pat. No. 8,734,809 SEQ ID NO: 15 |
| AAV CBr-E3 | 619 | U.S. Pat. No. 8,734,809 SEQ ID NO: 89 |
| AAV CBr-E4 | 620 | U.S. Pat. No. 8,734,809 SEQ ID NO: 16 |
| AAV CBr-E4 | 621 | U.S. Pat. No. 8,734,809 SEQ ID NO: 90 |
| AAV CBr-E5 | 622 | U.S. Pat. No. 8,734,809 SEQ ID NO: 17 |
| AAV CBr-e5 | 623 | U.S. Pat. No. 8,734,809 SEQ ID NO: 18 |
| AAV CBr-E5 | 624 | U.S. Pat. No. 8,734,809 SEQ ID NO: 91 |
| AAV CBr-e5 | 625 | U.S. Pat. No. 8,734,809 SEQ ID NO: 92 |
| AAV CBr-E6 | 626 | U.S. Pat. No. 8,734,809 SEQ ID NO: 19 |
| AAV CBr-E6 | 627 | U.S. Pat. No. 8,734,809 SEQ ID NO: 93 |
| AAV CBr-E7 | 628 | U.S. Pat. No. 8,734,809 SEQ ID NO: 20 |
| AAV CBr-E7 | 629 | U.S. Pat. No. 8,734,809 SEQ ID NO: 94 |
| AAV CBr-E8 | 630 | U.S. Pat. No. 8,734,809 SEQ ID NO: 21 |
| AAV CBr-E8 | 631 | U.S. Pat. No. 8,734,809 SEQ ID NO: 95 |
| AAV CHt-1 | 632 | U.S. Pat. No. 8,734,809 SEQ ID NO: 86 |
| AAV CHt-1 | 633 | U.S. Pat. No. 8,734,809 SEQ ID NO: 160 |
| AAV CHt-2 | 634 | U.S. Pat. No. 8,734,809 SEQ ID NO: 55 |
| AAV CHt-2 | 635 | U.S. Pat. No. 8,734,809 SEQ ID NO: 129 |
| AAV CHt-3 | 636 | U.S. Pat. No. 8,734,809 SEQ ID NO: 56 |
| AAV CHt-3 | 637 | U.S. Pat. No. 8,734,809 SEQ ID NO: 130 |
| AAV CHt-6.1 | 638 | WO2016065001 SEQ ID NO: 32 |
| AAV CHt-6.1 | 639 | WO2016065001 SEQ ID NO: 82 |
| AAV CHt-6.10 | 640 | WO2016065001 SEQ ID NO: 33 |
| AAV CHt-6.10 | 641 | WO2016065001 SEQ ID NO: 83 |
| AAV CHt-6.5 | 642 | WO2016065001 SEQ ID NO: 34 |
| AAV CHt-6.5 | 643 | WO2016065001 SEQ ID NO: 84 |
| AAV CHt-6.6 | 644 | WO2016065001 SEQ ID NO: 35 |
| AAV CHt-6.6 | 645 | WO2016065001 SEQ ID NO: 85 |
| AAV CHt-6.7 | 646 | WO2016065001 SEQ ID NO: 36 |
| AAV CHt-6.7 | 647 | WO2016065001 SEQ ID NO: 86 |
| AAV CHt-6.8 | 648 | WO2016065001 SEQ ID NO: 37 |
| AAV CHt-6.8 | 649 | WO2016065001 SEQ ID NO: 87 |
| AAV CHt-P1 | 650 | WO2016065001 SEQ ID NO: 29 |
| AAV CHt-P1 | 651 | WO2016065001 SEQ ID NO: 79 |
| AAV CHt-P2 | 652 | WO2016065001 SEQ ID NO: 1 |
| AAV CHt-P2 | 653 | WO2016065001 SEQ ID NO: 51 |
| AAV CHt-P5 | 654 | WO2016065001 SEQ ID NO: 2 |
| AAV CHt-P5 | 655 | WO2016065001 SEQ ID NO: 52 |
| AAV CHt-P6 | 656 | WO2016065001 SEQ ID NO: 30 |
| AAV CHt-P6 | 657 | WO2016065001 SEQ ID NO: 80 |
| AAV CHt-P8 | 658 | WO2016065001 SEQ ID NO: 31 |
| AAV CHt-P8 | 659 | WO2016065001 SEQ ID NO: 81 |
| AAV CHt-P9 | 660 | WO2016065001 SEQ ID NO: 3 |
| AAV CHt-P9 | 661 | WO2016065001 SEQ ID NO: 53 |
| AAV CKd-1 | 662 | U.S. Pat. No. 8,734,809 SEQ ID NO: 57 |
| AAV CKd-1 | 663 | U.S. Pat. No. 8,734,809 SEQ ID NO: 131 |
| AAV CKd-10 | 664 | U.S. Pat. No. 8,734,809 SEQ ID NO: 58 |
| AAV CKd-10 | 665 | U.S. Pat. No. 8,734,809 SEQ ID NO: 132 |
| AAV CKd-2 | 666 | U.S. Pat. No. 8,734,809 SEQ ID NO: 59 |
| AAV CKd-2 | 667 | U.S. Pat. No. 8,734,809 SEQ ID NO: 133 |
| AAV CKd-3 | 668 | U.S. Pat. No. 8,734,809 SEQ ID NO: 60 |
| AAV CKd-3 | 669 | U.S. Pat. No. 8,734,809 SEQ ID NO: 134 |
| AAV CKd-4 | 670 | U.S. Pat. No. 8,734,809 SEQ ID NO: 61 |
| AAV CKd-4 | 671 | U.S. Pat. No. 8,734,809 SEQ ID NO: 135 |
| AAV CKd-6 | 672 | U.S. Pat. No. 8,734,809 SEQ ID NO: 62 |
| AAV CKd-6 | 673 | U.S. Pat. No. 8,734,809 SEQ ID NO: 136 |
| AAV CKd-7 | 674 | U.S. Pat. No. 8,734,809 SEQ ID NO: 63 |
| AAV CKd-7 | 675 | U.S. Pat. No. 8,734,809 SEQ ID NO: 137 |
| AAV CKd-8 | 676 | U.S. Pat. No. 8,734,809 SEQ ID NO: 64 |
| AAV CKd-8 | 677 | U.S. Pat. No. 8,734,809 SEQ ID NO: 138 |
| AAV CKd-B1 | 678 | U.S. Pat. No. 8,734,809 SEQ ID NO: 73 |
| AAV CKd-B1 | 679 | U.S. Pat. No. 8,734,809 SEQ ID NO: 147 |
| AAV CKd-B2 | 680 | U.S. Pat. No. 8,734,809 SEQ ID NO: 74 |
| AAV CKd-B2 | 681 | U.S. Pat. No. 8,734,809 SEQ ID NO: 148 |
| AAV CKd-B3 | 682 | U.S. Pat. No. 8,734,809 SEQ ID NO: 75 |
| AAV CKd-B3 | 683 | U.S. Pat. No. 8,734,809 SEQ ID NO: 149 |
| AAV CKd-B4 | 684 | U.S. Pat. No. 8,734,809 SEQ ID NO: 76 |
| AAV CKd-B4 | 685 | U.S. Pat. No. 8,734,809 SEQ ID NO: 150 |
| AAV CKd-B5 (AAV CKd- | 686 | U.S. Pat. No. 8,734,809 SEQ ID NO: 77 |
| H6) | | |
| AAV CKd-B5 (AAV CKd- | 687 | U.S. Pat. No. 8,734,809 SEQ ID NO: 151 |
| H6) | | |
| AAV CKd-B6 | 688 | U.S. Pat. No. 8,734,809 SEQ ID NO: 78 |
| AAV CKd-B6 | 689 | U.S. Pat. No. 8,734,809 SEQ ID NO: 152 |
| AAV CKd-B7 | 690 | U.S. Pat. No. 8,734,809 SEQ ID NO: 79 |
| AAV CKd-B7 | 691 | U.S. Pat. No. 8,734,809 SEQ ID NO: 153 |
| AAV CKd-B8 | 692 | U.S. Pat. No. 8,734,809 SEQ ID NO: 80 |
| AAV CKd-B8 | 693 | U.S. Pat. No. 8,734,809 SEQ ID NO: 154 |
| AAV CKd-H1 | 694 | U.S. Pat. No. 8,734,809 SEQ ID NO: 81 |
| AAV CKd-H1 | 695 | U.S. Pat. No. 8,734,809 SEQ ID NO: 155 |
| AAV CKd-H2 | 696 | U.S. Pat. No. 8,734,809 SEQ ID NO: 82 |
| AAV CKd-H2 | 697 | U.S. Pat. No. 8,734,809 SEQ ID NO: 156 |
| AAV CKd-H3 | 698 | U.S. Pat. No. 8,734,809 SEQ ID NO: 83 |
| AAV CKd-H3 | 699 | U.S. Pat. No. 8,734,809 SEQ ID NO: 157 |
| AAV CKd-H4 | 700 | U.S. Pat. No. 8,734,809 SEQ ID NO: 84 |
| AAV CKd-H4 | 701 | U.S. Pat. No. 8,734,809 SEQ ID NO: 158 |
| AAV CKd-H5 | 702 | U.S. Pat. No. 8,734,809 SEQ ID NO: 85 |
| AAV CKd-H5 | 703 | U.S. Pat. No. 8,734,809 SEQ ID NO: 159 |
| AAV CKd-N3 | 704 | WO2016065001 SEQ ID NO: 12 |
| AAV CKd-N3 | 705 | WO2016065001 SEQ ID NO: 62 |
| AAV CKd-N4 | 706 | WO2016065001 SEQ ID NO: 13 |
| AAV CKd-N4 | 707 | WO2016065001 SEQ ID NO: 63 |
| AAV CKd-N9 | 708 | WO2016065001 SEQ ID NO: 14 |
| AAV CKd-N9 | 709 | WO2016065001 SEQ ID NO: 64 |
| AAV CLg-F1 | 710 | U.S. Pat. No. 8,734,809 SEQ ID NO: 39 |
| AAV CLg-F1 | 711 | U.S. Pat. No. 8,734,809 SEQ ID NO: 113 |
| AAV CLg-F2 | 712 | U.S. Pat. No. 8,734,809 SEQ ID NO: 40 |
| AAV CLg-F2 | 713 | U.S. Pat. No. 8,734,809 SEQ ID NO: 114 |
| AAV CLg-F3 | 714 | U.S. Pat. No. 8,734,809 SEQ ID NO: 41 |
| AAV CLg-F3 | 715 | U.S. Pat. No. 8,734,809 SEQ ID NO: 115 |
| AAV CLg-F4 | 716 | U.S. Pat. No. 8,734,809 SEQ ID NO: 42 |
| AAV CLg-F4 | 717 | U.S. Pat. No. 8,734,809 SEQ ID NO: 116 |
| AAV CLg-F5 (AAV CLg- | 718 | U.S. Pat. No. 8,734,809 SEQ ID NO: 43 |
| F6; AAV CLg-F8) | | |
| AAV CLg-F5 (AAV CLg- | 719 | U.S. Pat. No. 8,734,809 SEQ ID NO: 117 |
| F6; AAV CLg-F8) | | |
| AAV CLg-F7 | 720 | U.S. Pat. No. 8,734,809 SEQ ID NO: 44 |
| AAV CLg-F7 | 721 | U.S. Pat. No. 8,734,809 SEQ ID NO: 118 |
| AAV CLv-1 | 722 | U.S. Pat. No. 8,734,809 SEQ ID NO: 65 |
| AAV CLv-1 | 723 | U.S. Pat. No. 8,734,809 SEQ ID NO: 139 |
| AAV CLv1-1 | 724 | U.S. Pat. No. 8,734,809 SEQ ID NO: 171 |
| AAV Clv1-10 | 725 | U.S. Pat. No. 8,734,809 SEQ ID NO: 178 |
| AAV CLv1-2 | 726 | U.S. Pat. No. 8,734,809 SEQ ID NO: 172 |
| AAV CLv-12 | 727 | U.S. Pat. No. 8,734,809 SEQ ID NO: 66 |
| AAV CLv-12 | 728 | U.S. Pat. No. 8,734,809 SEQ ID NO: 140 |
| AAV CLv1-3 | 729 | U.S. Pat. No. 8,734,809 SEQ ID NO: 173 |
| AAV CLv-13 | 730 | U.S. Pat. No. 8,734,809 SEQ ID NO: 67 |
| AAV CLv-13 | 731 | U.S. Pat. No. 8,734,809 SEQ ID NO: 141 |
| AAV CLv1-4 | 732 | U.S. Pat. No. 8,734,809 SEQ ID NO: 174 |
| AAV Clv1-7 | 733 | U.S. Pat. No. 8,734,809 SEQ ID NO: 175 |
| AAV Clv1-8 | 734 | U.S. Pat. No. 8,734,809 SEQ ID NO: 176 |
| AAV Clv1-9 | 735 | U.S. Pat. No. 8,734,809 SEQ ID NO: 177 |
| AAV CLv-2 | 736 | U.S. Pat. No. 8,734,809 SEQ ID NO: 68 |
| AAV CLv-2 | 737 | U.S. Pat. No. 8,734,809 SEQ ID NO: 142 |
| AAV CLv-3 | 738 | U.S. Pat. No. 8,734,809 SEQ ID NO: 69 |
| AAV CLv-3 | 739 | U.S. Pat. No. 8,734,809 SEQ ID NO: 143 |
| AAV CLv-4 | 740 | U.S. Pat. No. 8,734,809 SEQ ID NO: 70 |
| AAV CLv-4 | 741 | U.S. Pat. No. 8,734,809 SEQ ID NO: 144 |
| AAV CLv-6 | 742 | U.S. Pat. No. 8,734,809 SEQ ID NO: 71 |
| AAV CLv-6 | 743 | U.S. Pat. No. 8,734,809 SEQ ID NO: 145 |
| AAV CLv-8 | 744 | U.S. Pat. No. 8,734,809 SEQ ID NO: 72 |
| AAV CLv-8 | 745 | U.S. Pat. No. 8,734,809 SEQ ID NO: 146 |
| AAV CLv-D1 | 746 | U.S. Pat. No. 8,734,809 SEQ ID NO: 22 |
| AAV CLv-D1 | 747 | U.S. Pat. No. 8,734,809 SEQ ID NO: 96 |
| AAV CLv-D2 | 748 | U.S. Pat. No. 8,734,809 SEQ ID NO: 23 |
| AAV CLv-D2 | 749 | U.S. Pat. No. 8,734,809 SEQ ID NO: 97 |
| AAV CLv-D3 | 750 | U.S. Pat. No. 8,734,809 SEQ ID NO: 24 |
| AAV CLv-D3 | 751 | U.S. Pat. No. 8,734,809 SEQ ID NO: 98 |
| AAV CLv-D4 | 752 | U.S. Pat. No. 8,734,809 SEQ ID NO: 25 |
| AAV CLv-D4 | 753 | U.S. Pat. No. 8,734,809 SEQ ID NO: 99 |
| AAV CLv-D5 | 754 | U.S. Pat. No. 8,734,809 SEQ ID NO: 26 |
| AAV CLv-D5 | 755 | U.S. Pat. No. 8,734,809 SEQ ID NO: 100 |
| AAV CLv-D6 | 756 | U.S. Pat. No. 8,734,809 SEQ ID NO: 27 |
| AAV CLv-D6 | 757 | U.S. Pat. No. 8,734,809 SEQ ID NO: 101 |
| AAV CLv-D7 | 758 | U.S. Pat. No. 8,734,809 SEQ ID NO: 28 |
| AAV CLv-D7 | 759 | U.S. Pat. No. 8,734,809 SEQ ID NO: 102 |
| AAV CLv-D8 | 760 | U.S. Pat. No. 8,734,809 SEQ ID NO: 29 |
| AAV CLv-D8 | 761 | U.S. Pat. No. 8,734,809 SEQ ID NO: 103 |
| AAV CLv-K1 | 762 | WO2016065001 SEQ ID NO: 18 |
| AAV CLv-K1 | 763 | WO2016065001 SEQ ID NO: 68 |
| AAV CLv-K3 | 764 | WO2016065001 SEQ ID NO: 19 |
| AAV CLv-K3 | 765 | WO2016065001 SEQ ID NO: 69 |
| AAV CLv-K6 | 766 | WO2016065001 SEQ ID NO: 20 |
| AAV CLv-K6 | 767 | WO2016065001 SEQ ID NO: 70 |
| AAV CLv-L4 | 768 | WO2016065001 SEQ ID NO: 15 |
| AAV CLv-L4 | 769 | WO2016065001 SEQ ID NO: 65 |
| AAV CLv-L5 | 770 | WO2016065001 SEQ ID NO: 16 |
| AAV CLv-L5 | 771 | WO2016065001 SEQ ID NO: 66 |
| AAV CLv-L6 | 772 | WO2016065001 SEQ ID NO: 17 |
| AAV CLv-L6 | 773 | WO2016065001 SEQ ID NO: 67 |
| AAV CLv-M1 | 774 | WO2016065001 SEQ ID NO: 21 |
| AAV CLv-M1 | 775 | WO2016065001 SEQ ID NO: 71 |
| AAV CLv-M11 | 776 | WO2016065001 SEQ ID NO: 22 |
| AAV CLv-M11 | 777 | WO2016065001 SEQ ID NO: 72 |
| AAV CLv-M2 | 778 | WO2016065001 SEQ ID NO: 23 |
| AAV CLv-M2 | 779 | WO2016065001 SEQ ID NO: 73 |
| AAV CLv-M5 | 780 | WO2016065001 SEQ ID NO: 24 |
| AAV CLv-M5 | 781 | WO2016065001 SEQ ID NO: 74 |
| AAV CLv-M6 | 782 | WO2016065001 SEQ ID NO: 25 |
| AAV CLv-M6 | 783 | WO2016065001 SEQ ID NO: 75 |
| AAV CLv-M7 | 784 | WO2016065001 SEQ ID NO: 26 |
| AAV CLv-M7 | 785 | WO2016065001 SEQ ID NO: 76 |
| AAV CLv-M8 | 786 | WO2016065001 SEQ ID NO: 27 |
| AAV CLv-M8 | 787 | WO2016065001 SEQ ID NO: 77 |
| AAV CLv-M9 | 788 | WO2016065001 SEQ ID NO: 28 |
| AAV CLv-M9 | 789 | WO2016065001 SEQ ID NO: 78 |
| AAV CLv-R1 | 790 | U.S. Pat. No. 8,734,809 SEQ ID NO: 30 |
| AAV CLv-R1 | 791 | U.S. Pat. No. 8,734,809 SEQ ID NO: 104 |
| AAV CLv-R2 | 792 | U.S. Pat. No. 8,734,809 SEQ ID NO: 31 |
| AAV CLv-R2 | 793 | U.S. Pat. No. 8,734,809 SEQ ID NO: 105 |
| AAV CLv-R3 | 794 | U.S. Pat. No. 8,734,809 SEQ ID NO: 32 |
| AAV CLv-R3 | 795 | U.S. Pat. No. 8,734,809 SEQ ID NO: 106 |
| AAV CLv-R4 | 796 | U.S. Pat. No. 8,734,809 SEQ ID NO: 33 |
| AAV CLv-R4 | 797 | U.S. Pat. No. 8,734,809 SEQ ID NO: 107 |
| AAV CLv-R5 | 798 | U.S. Pat. No. 8,734,809 SEQ ID NO: 34 |
| AAV CLv-R5 | 799 | U.S. Pat. No. 8,734,809 SEQ ID NO: 108 |
| AAV CLv-R6 | 800 | U.S. Pat. No. 8,734,809 SEQ ID NO: 35 |
| AAV CLv-R6 | 801 | U.S. Pat. No. 8,734,809 SEQ ID NO: 109 |
| AAV CLv-R7 | 802 | U.S. Pat. No. 8,734,809 SEQ ID NO: 36 |
| AAV CLv-R7 | 803 | U.S. Pat. No. 8,734,809 SEQ ID NO: 110 |
| AAV CLv-R8 | 804 | U.S. Pat. No. 8,734,809 SEQ ID NO: 37 |
| AAV CLv-R8 | 805 | U.S. Pat. No. 8,734,809 SEQ ID NO: 111 |
| AAV CLv-R9 | 806 | U.S. Pat. No. 8,734,809 SEQ ID NO: 38 |
| AAV CLv-R9 | 807 | U.S. Pat. No. 8,734,809 SEQ ID NO: 112 |
| AAV CSp-1 | 808 | U.S. Pat. No. 8,734,809 SEQ ID NO: 45 |
| AAV CSp-1 | 809 | U.S. Pat. No. 8,734,809 SEQ ID NO: 119 |
| AAV CSp-10 | 810 | U.S. Pat. No. 8,734,809 SEQ ID NO: 46 |
| AAV CSp-10 | 811 | U.S. Pat. No. 8,734,809 SEQ ID NO: 120 |
| AAV CSp-11 | 812 | U.S. Pat. No. 8,734,809 SEQ ID NO: 47 |
| AAV CSp-11 | 813 | U.S. Pat. No. 8,734,809 SEQ ID NO: 121 |
| AAV CSp-2 | 814 | U.S. Pat. No. 8,734,809 SEQ ID NO: 48 |
| AAV CSp-2 | 815 | U.S. Pat. No. 8,734,809 SEQ ID NO: 122 |
| AAV CSp-3 | 816 | U.S. Pat. No. 8,734,809 SEQ ID NO: 49 |
| AAV CSp-3 | 817 | U.S. Pat. No. 8,734,809 SEQ ID NO: 123 |
| AAV CSp-4 | 818 | U.S. Pat. No. 8,734,809 SEQ ID NO: 50 |
| AAV CSp-4 | 819 | U.S. Pat. No. 8,734,809 SEQ ID NO: 124 |
| AAV CSp-6 | 820 | U.S. Pat. No. 8,734,809 SEQ ID NO: 51 |
| AAV CSp-6 | 821 | U.S. Pat. No. 8,734,809 SEQ ID NO: 125 |
| AAV CSp-7 | 822 | U.S. Pat. No. 8,734,809 SEQ ID NO: 52 |
| AAV CSp-7 | 823 | U.S. Pat. No. 8,734,809 SEQ ID NO: 126 |
| AAV CSp-8 | 824 | U.S. Pat. No. 8,734,809 SEQ ID NO: 53 |
| AAV CSp-8 | 825 | U.S. Pat. No. 8,734,809 SEQ ID NO: 127 |
| AAV CSp-8.10 | 826 | WO2016065001 SEQ ID NO: 38 |
| AAV CSp-8.10 | 827 | WO2016065001 SEQ ID NO: 88 |
| AAV CSp-8.2 | 828 | WO2016065001 SEQ ID NO: 39 |
| AAV CSp-8.2 | 829 | WO2016065001 SEQ ID NO: 89 |
| AAV CSp-8.4 | 830 | WO2016065001 SEQ ID NO: 40 |
| AAV CSp-8.4 | 831 | WO2016065001 SEQ ID NO: 90 |
| AAV CSp-8.5 | 832 | WO2016065001 SEQ ID NO: 41 |
| AAV CSp-8.5 | 833 | WO2016065001 SEQ ID NO: 91 |
| AAV CSp-8.6 | 834 | WO2016065001 SEQ ID NO: 42 |
| AAV CSp-8.6 | 835 | WO2016065001 SEQ ID NO: 92 |
| AAV CSp-8.7 | 836 | WO2016065001 SEQ ID NO: 43 |
| AAV CSp-8.7 | 837 | WO2016065001 SEQ ID NO: 93 |
| AAV CSp-8.8 | 838 | WO2016065001 SEQ ID NO: 44 |
| AAV CSp-8.8 | 839 | WO2016065001 SEQ ID NO: 94 |
| AAV CSp-8.9 | 840 | WO2016065001 SEQ ID NO: 45 |
| AAV CSp-8.9 | 841 | WO2016065001 SEQ ID NO: 95 |
| AAV CSp-9 | 842 | U.S. Pat. No. 8,734,809 SEQ ID NO: 54 |
| AAV CSp-9 | 843 | U.S. Pat. No. 8,734,809 SEQ ID NO: 128 |
| AAV.hu.48R3 | 844 | U.S. Pat. No. 8,734,809 SEQ ID NO: 183 |
| AAV.VR-355 | 845 | U.S. Pat. No. 8,734,809 SEQ ID NO: 181 |
| AAV3B | 846 | WO2016065001 SEQ ID NO: 48 |
| AAV3B | 847 | WO2016065001 SEQ ID NO: 98 |
| AAV4 | 848 | WO2016065001 SEQ ID NO: 49 |
| AAV4 | 849 | WO2016065001 SEQ ID NO: 99 |
| AAV5 | 850 | WO2016065001 SEQ ID NO: 50 |
| AAV5 | 851 | WO2016065001 SEQ ID NO: 100 |
| AAVF1/HSC1 | 852 | WO2016049230 SEQ ID NO: 20 |
| AAVF1/HSC1 | 853 | WO2016049230 SEQ ID NO: 2 |
| AAVF11/HSC11 | 854 | WO2016049230 SEQ ID NO: 26 |
| AAVF11/HSC11 | 855 | WO2016049230 SEQ ID NO: 4 |
| AAVF12/HSC12 | 856 | WO2016049230 SEQ ID NO: 30 |
| AAVF12/HSC12 | 857 | WO2016049230 SEQ ID NO: 12 |
| AAVF13/HSC13 | 858 | WO2016049230 SEQ ID NO: 31 |
| AAVF13/HSC13 | 859 | WO2016049230 SEQ ID NO: 14 |
| AAVF14/HSC14 | 860 | WO2016049230 SEQ ID NO: 32 |
| AAVF14/HSC14 | 861 | WO2016049230 SEQ ID NO: 15 |
| AAVF15/HSC15 | 862 | WO2016049230 SEQ ID NO: 33 |
| AAVF15/HSC15 | 863 | WO2016049230 SEQ ID NO: 16 |
| AAVF16/HSC16 | 864 | WO2016049230 SEQ ID NO: 34 |
| AAVF16/HSC16 | 865 | WO2016049230 SEQ ID NO: 17 |
| AAVF17/HSC17 | 866 | WO2016049230 SEQ ID NO: 35 |
| AAVF17/HSC17 | 867 | WO2016049230 SEQ ID NO: 13 |
| AAVF2/HSC2 | 868 | WO2016049230 SEQ ID NO: 21 |
| AAVF2/HSC2 | 869 | WO2016049230 SEQ ID NO: 3 |
| AAVF3/HSC3 | 870 | WO2016049230 SEQ ID NO: 22 |
| AAVF3/HSC3 | 871 | WO2016049230 SEQ ID NO: 5 |
| AAVF4/HSC4 | 872 | WO2016049230 SEQ ID NO: 23 |
| AAVF4/HSC4 | 873 | WO2016049230 SEQ ID NO: 6 |
| AAVF5/HSC5 | 874 | WO2016049230 SEQ ID NO: 25 |
| AAVF5/HSC5 | 875 | WO2016049230 SEQ ID NO: 11 |
| AAVF6/HSC6 | 876 | WO2016049230 SEQ ID NO: 24 |
| AAVF6/HSC6 | 877 | WO2016049230 SEQ ID NO: 7 |
| AAVF7/HSC7 | 878 | WO2016049230 SEQ ID NO: 27 |
| AAVF7/HSC7 | 879 | WO2016049230 SEQ ID NO: 8 |
| AAVF8/HSC8 | 880 | WO2016049230 SEQ ID NO: 28 |
| AAVF8/HSC8 | 881 | WO2016049230 SEQ ID NO: 9 |
| AAVF9/HSC9 | 882 | WO2016049230 SEQ ID NO: 29 |
| AAVF9/HSC9 | 883 | WO2016049230 SEQ ID NO: 10 |

Each of the patents, applications and/or publications listed in Table 1 are hereby incorporated by reference in their entirety.

In one embodiment, the AAV particle may be engineered to comprise at least one AAV capsid CD8+ T-cell epitope. Hui et al. (Molecular Therapy—Methods & Clinical Development (2015) 2, 15029 doi:10.1038/mtm.2015.29; the contents of which are herein incorporated by reference in their entirety) identified AAV capsid-specific CD8+ T-cell epitopes for AAV1 and AAV2 (see e.g., Table 2 in the publication). As a non-limiting example, the capsid-specific CD8+ T-cell epitope may be for an AAV2 serotype. As a non-limiting example, the capsid-specific CD8+ T-cell epitope may be for an AAV1 serotype.

In one embodiment, the AAV particle may be engineered to comprise at least one AAV capsid CD8+ T-cell epitope for AAV2 such as, but not limited to, SADNNNSEY (SEQ ID NO: 884), LIDQYLYYL (SEQ ID NO: 885), VPQYGYLTL (SEQ ID NO: 886), TTSTRTWAL (SEQ ID NO: 887), YHLNGRDSL (SEQ ID NO: 888), SQAVGRSSF (SEQ ID NO: 889), VPANPSTTF (SEQ ID NO: 890), FPQSGVLIF (SEQ ID NO: 891), YFDFNRFHCHFSPRD (SEQ ID NO: 892), VGNSSGNWHCDSTWM (SEQ ID NO: 893), QFSQAGASDIRDQSR (SEQ ID NO: 894), GASDIRQSRNWLP (SEQ ID NO: 895) and GNRQAATADVNTQGV (SEQ ID NO: 896).

In one embodiment, the AAV particle may be engineered to comprise at least one AAV capsid CD8+ T-cell epitope for AAV1 such as, but not limited to, LDRLMNPLI (SEQ ID NO: 897), TTSTRTWAL (SEQ ID NO: 887), and QPAKKRLNF (SEQ ID NO: 898).

In one embodiment, peptides for inclusion in an AAV particle may be identified using the methods described by Hui et al. (Molecular Therapy—Methods & Clinical Development (2015) 2, 15029 doi:10.1038/mtm.2015.29; the contents of which are herein incorporated by reference in their entirety). As a non-limiting example, the procedure includes isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the HLA restriction of 15-mers identified by IFN-gamma ELISpot, identification of candidate reactive 9-mer epitopes for a given HLA allele, synthesis candidate 9-mers, second IFN-gamma ELISpot screening of splenocytes from subjects carrying the HLA alleles to which identified AAV epitopes are predicted to bind, determine the AAV capsid-reactive CD8+ T cell epitopes and determine the frequency of subjects reacting to a given AAV epitope.

In one embodiment, peptides for inclusion in an AAV particle may be identified by isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the given allele restriction of 15-mers identified by IFN-gamma ELISpot, identification of candidate reactive 9-mer epitopes for a given allele, synthesis candidate 9-mers, second IFN-gamma ELISpot screening of splenocytes from subjects carrying the specific alleles to which identified AAV epitopes are predicted to bind, determine the AAV capsid-reactive CD8+ T cell epitopes and determine the frequency of subjects reacting to a given AAV epitope.

### AAV Vectors and Capsids

The present disclosure also provides nucleic acids encoding the mutated or modified virus capsids and capsid proteins. In some embodiments the capsids are engineered according to the methods of International Publication No. WO2015191508, the contents of which are incorporated herein by reference in their entirety and by methods known in the art.

Further provided are vectors comprising the nucleic acids, and cells (in vivo or in culture) comprising the nucleic acids and/or vectors. Suitable vectors include without limitation viral vectors (e.g., adenovirus, AAV, herpes virus, vaccinia, poxviruses, baculoviruses, and the like), plasmids, phage, YACs, BACs, and the like as are well known in the art. Such nucleic acids, vectors and cells can be used, for example, as reagents (e.g., helper packaging constructs or packaging cells) for the production of modified virus capsids or virus vectors as described herein.

The molecules which contain AAV sequences include any genetic element (vector) which may be delivered to a host cell, e.g., naked DNA, a plasmid, phage, transposon, cosmid, episome, a protein in a non-viral delivery vehicle (e.g., a lipid-based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this disclosure are known to those with skill in nucleic acid manipulation and include genetic engineering, recombinant engineering, and synthetic techniques. (See, e.g., Sambrook et al., Molecular Cloning: A Laboratory Manual, Cold Spring Harbor Press, Cold Spring Harbor, N.Y.).

The transgene or payload can be carried on any suitable vector, e.g., a plasmid, which is delivered to a host cell. The plasmids useful in this disclosure may be engineered such that they are suitable for replication and, optionally, integration in prokaryotic cells, mammalian cells, or both. These plasmids contain sequences permitting replication of the transgene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the transgene or payload is transfected into the cell, where it may exist transiently. Alternatively, the transgene may be stably integrated into the genome of the host cell, either chromosomally or as an episome. In certain embodiments, the transgene may be present in multiple copies, optionally in head-to-head, head-to-tail, or tail-to-tail concatamers. Suitable transfection techniques are known and may readily be utilized to deliver the transgene to the host cell.

### Promoters

A person skilled in the art may recognize that a target cell may require a specific promoter including but not limited to a promoter that is species specific, inducible, tissue-specific, or cell cycle-specific Parr et al., Nat. Med.3:1145-9 (1997); the contents of which are herein incorporated by reference in their entirety.

In one embodiment, the promoter is deemed to be efficient for the AADC polynucleotide.

In one embodiment, the promoter is deemed to be efficient when it drives expression of the polypeptide(s) encoded in the payload region of the viral genome of the AAV particle.

In one embodiment, the promoter is deemed to be efficient for the cell being targeted.

In one embodiment, the promoter provides expression of a payload described herein for a period of time in targeted tissues. As a non-limiting example, expression driven by a promoter may be for nervous system tissues. Expression may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a promoter for sustained expression in nervous system tissue such as, but not limited to, neuronal tissue and glial tissue.

In one embodiment, the promoter is a promoter which provides expression of a payload described herein for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

In one embodiment, the FRDA promoter is used with the AADC polynucleotides described herein.

In one embodiment, there is a region located approximately ˜5 kb upstream of the first exon of the payload. As a non-limiting example, there is a 17 bp region located approximately 4.9 kb upstream of the first exon of the frataxin gene in order to allow for expression with the FRDA promoter (See e.g., Puspasari et al. Long Range Regulation of Human FXN Gene Expression, PLOS ONE, 2011; the contents of which are herein incorporated by reference in their entirety).

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, 0 glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In one embodiment, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In one embodiment, the promoter is a combination of a 382 nucleotide CMV-enhancer sequence and a 260 nucleotide CBA-promoter sequence.

In one embodiment, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIa, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in their entirety) evaluated the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in their entirety) evaluated a HβH construct with a hGUSB promoter, a HSV-1LAT promoter and a NSE promoter and found that the HβH construct showed weaker expression than NSE in mouse brain. Passini and Wolfe (J. Virol. 2001, 12382-12392, the contents of which are herein incorporated by reference in their entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews or Raymond may be used in the present inventions.

In one embodiment, the promoter is not cell specific.

In one embodiment, the promoter is an ubiquitin c (UBC) promoter. The UBC promoter may have a size of 300-350 nucleotides. As a non-limiting example, the UBC promoter is 332 nucleotides.

In one embodiment, the promoter is a 3-glucuronidase (GUSB) promoter. The GUSB promoter may have a size of 350-400 nucleotides. As a non-limiting example, the GUSB promoter is 378 nucleotides.

In one embodiment, the promoter is a neurofilament light (NFL) promoter. The NFL promoter may have a size of 600-700 nucleotides. As a non-limiting example, the NFL promoter is 650 nucleotides.

In one embodiment, the promoter is a neurofilament heavy (NFH) promoter. The NFH promoter may have a size of 900-950 nucleotides. As a non-limiting example, the NFH promoter is 920 nucleotides.

In one embodiment, the promoter is a scn8a promoter. The scn8a promoter may have a size of 450-500 nucleotides. As a non-limiting example, the scn8a promoter is 470 nucleotides.

In one embodiment, the promoter is a phosphoglycerate kinase 1 (PGK) promoter.

In one embodiment, the promoter is a chicken β-actin (CBA) promoter.

In one embodiment, the promoter is a cytomegalovirus (CMV) promoter.

In one embodiment, the promoter is a neuronal cell targeting promoter. A non-limiting example of a neuronal targeting promoter is a synapsin-1 promoter.

In one embodiment, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In one embodiment, the promoter is a RNA pol III promoter. As a non-limiting example, the RNA pol III promoter is U6. As a non-limiting example, the RNA pol III promoter is H1.

In one embodiment, the viral genome comprises two promoters. As a non-limiting example, the promoters are an EF1α promoter and a CMV promoter.

In one embodiment, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In one embodiment, the viral genome comprises an engineered promoter.

In another embodiment the viral genome comprises a promoter from a naturally expressed protein.

### Introns

In one embodiment, at least one element may be used with the AADC polynucleotides described herein to enhance the transgene target specificity and expression (See e.g., Powell et al. Viral Expression Cassette Elements to Enhance Transgene Target Specificity and Expression in Gene Therapy, 2015; the contents of which are herein incorporated by reference in their entirety) such as an intron. Non-limiting examples of introns include, MVM (67-97 bps), F.IX truncated intron 1 (300 bps), 3-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

### Functional Payloads

A payload may comprise a gene therapy product. A gene therapy product may comprise a polypeptide, RNA molecule, or other gene product that, when expressed in a target cell, provides a desired therapeutic effect. In some embodiments, a gene therapy product may comprise a substitute for a non-functional gene that is absent or mutated.

An AAV payload construct encoding a payload may comprise a selectable marker. A selectable marker may comprise a gene sequence or a protein encoded by that gene sequence expressed in a host cell that allows for the identification, selection, and/or purification of the host cell from a population of cells that may or may not express the selectable marker. In one embodiment the selectable marker provides resistance to survive a selection process that would otherwise kill the host cell, such as treatment with an antibiotic. In some embodiments an antibiotic selectable marker may comprise one or more antibiotic resistance factors, including but not limited to neomycin resistance (e.g., neo), hygromycin resistance, kanamycin resistance, and/or puromycin resistance.

In some embodiments a selectable marker may comprise a cell-surface marker, such as any protein expressed on the surface of the cell including, but not limited to receptors, CD markers, lectins, integrins, or truncated versions thereof. In some embodiments, cells that comprise a cell-surface marker may be selected using an antibody targeted to said cell-surface marker. In some embodiments an antibody targeted to the cell-surface marker may be directly conjugated with a selection agent including, but not limited to a fluorophore, sepharose, or magnetic bead. In some embodiments an antibody targeted to the cell-surface marker may be detected using a secondary labeled antibody or substrate which binds to the antibody targeted to the cell-surface marker. In some embodiments, a selectable marker may comprise negative selection by using an enzyme, including but not limited to Herpes simplex virus thymidine kinase (HSVTK) that converts a pro-toxin (gancyclovir) into a toxin or bacterial Cytosine Deaminase (CD) which converts the pro-toxin 5′-fluorocytosine (5′-FC) into the toxin 5′-fluorouracil (5′-FU). In some embodiments, any nucleic acid sequence encoding a polypeptide can be used as a selectable marker comprising recognition by a specific antibody.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540). In some embodiments, a nucleic acid encoding a selectable marker may comprise a fluorescent protein. A fluorescent protein as herein described may comprise any fluorescent marker including but not limited to green, yellow, and/or red fluorescent protein (GFP, YFP, and/or RFP).

In accordance with the invention, a payload comprising a nucleic acid for expression in a target cell will be incorporated into the viral particle produced in the viral replication cell where said payload is located between two ITR sequences, or is located on either side of an asymmetrical ITR engineered with two D regions.

An AAV payload construct encoding one or more payloads for expression in a target cell may comprise one or more payload or non-payload nucleotide sequences operably linked to at least one target cell-compatible promoter. A person skilled in the art may recognize that a target cell may require a specific promoter including but not limited to a promoter that is species specific, inducible, tissue-specific, or cell cycle-specific Parr et al., Nat. Med.3:1145-9 (1997).

### Viral Production

The present disclosure provides a method for the generation of parvoviral particles, e.g. AAV particles, by viral genome replication in a viral replication cell comprising contacting the viral replication cell with an AAV polynucleotide or AAV genome.

In one embodiment, the AAV particle of the present invention may be produced in insect cells (e.g., Sf9 cells).

In one embodiment, the AAV particle of the present invention may be produced using triple transfection.

In one embodiment, the AAV particle of the present invention may be produced in mammalian cells.

In one embodiment, the AAV particle of the present invention may be produced by triple transfection in mammalian cells.

In one embodiment, the AAV particle of the present invention may be produced by triple transfection in HEK293 cells.

In some embodiments, the present invention provides a method for producing an AAV particle having enhanced (increased, improved) transduction efficiency comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying the AAV particle comprising a viral genome.

In some embodiments, the present invention provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

### Cells

The present disclosure provides a cell comprising an AAV polynucleotide and/or AAV genome.

Viral production disclosed herein describes processes and methods for producing AAV particles that contact a target cell to deliver a payload construct, e.g. a recombinant viral construct, which comprises a nucleotide encoding a payload molecule.

In one embodiment, the AAV particles may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present invention. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in their entirety.

The viral replication cell may be selected from any biological organism, including prokaryotic (e.g., bacterial) cells, and eukaryotic cells, including, insect cells, yeast cells and mammalian cells. Viral replication cells may comprise mammalian cells such as A549, WEH1, 3T3, 10T1/2, BHK, MDCK, COS 1, COS 7, BSC 1, BSC 40, BMT 10, VERO, W138, HeLa, 293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

### Small Scale Production of AAV Particles

Viral production disclosed herein describes processes and methods for producing AAV particles that contact a target cell to deliver a payload, e.g. a recombinant viral construct, which comprises a nucleotide encoding a payload.

In one embodiment, the AAV particles may be produced in a viral replication cell that comprises a mammalian cell.

Viral replication cells commonly used for production of recombinant AAV particles include, but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent application 2002/0081721, and International Patent Applications WO 00/47757, WO 00/24916, and WO 96/17947, the contents of each of which are herein incorporated by reference in their entireties.

In one embodiment, AAV particles are produced in mammalian-cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In another embodiment, AAV particles are produced in mammalian cells using a triple transfection method wherein a payload construct, parvoviral Rep, and parvoviral Cap are comprised within three different constructs. The triple transfection method of the three components of AAV particle production may be utilized to produce small lots of virus for assays including transduction efficiency, target tissue (tropism) evaluation, and stability.

### Baculovirus

Particle production disclosed herein describes processes and methods for producing AAV particles that contact a target cell to deliver a payload construct which comprises a nucleotide encoding a payload.

Briefly, the viral construct vector and the AAV payload construct vector are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses may be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al., J Virol. 2006 February; 80 (4): 1874-85, the contents of which are herein incorporated by reference in their entirety.

Production of AAV particles with baculovirus in an insect cell system may address known baculovirus genetic and physical instability. In one embodiment, the production system addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65(2): 122-32, the contents of which are herein incorporated by reference in their entirety.

A genetically stable baculovirus may be used to produce source of the one or more of the components for producing AAV particles in invertebrate cells. In one embodiment, defective baculovirus expression vectors may be maintained episomally in insect cells. In such an embodiment the bacmid vector is engineered with replication control elements, including but not limited to promoters, enhancers, and/or cell-cycle regulated replication elements.

In one embodiment, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In one embodiment, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

### Large-Scale Production

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (Thermo Scientific, Waltham, Mass.) In some cases, large-scale adherent cell surfaces may comprise from about 1,000 cm² to about 100,000 cm². In some cases, large-scale adherent cell cultures may comprise from about 10⁹ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds (e.g. polyethylenimine (PEI)) or the use of non-chemical methods (e.g. electroporation.) With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more AAV payload construct. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

### Bioreactors

In some embodiments, cell culture bioreactors may be used for large scale viral production. In some cases, bioreactors comprise stirred tank reactors. Such reactors generally comprise a vessel, typically cylindrical in shape, with a stirrer (e.g. impeller.) In some embodiments, such bioreactor vessels may be placed within a water jacket to control vessel temperature and/or to minimize effects from ambient temperature changes. Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In some cases, animal cell cultures may be maintained in bioreactors with rounded vessel bottoms.

In some cases, bioreactor vessels may be warmed through the use of a thermocirculator. Thermocirculators pump heated water around water jackets. In some cases, heated water may be pumped through pipes (e.g. coiled pipes) that are present within bioreactor vessels. In some cases, warm air may be circulated around bioreactors, including, but not limited to air space directly above culture medium. Additionally, pH and C02 levels may be maintained to optimize cell viability.

In some cases, bioreactors may comprise hollow-fiber reactors. Hollow-fiber bioreactors may support the culture of both anchorage dependent and anchorage independent cells. Further bioreactors may include, but are not limited to packed-bed or fixed-bed bioreactors. Such bioreactors may comprise vessels with glass beads for adherent cell attachment. Further packed-bed reactors may comprise ceramic beads.

In some cases, viral particles are produced through the use of a disposable bioreactor. In some embodiments, such bioreactors may include WAVE™ disposable bioreactors.

In some embodiments, AAV particle production in animal cell bioreactor cultures may be carried out according to the methods taught in U.S. Pat. Nos. 5,064,764, 6,194,191, 6,566,118, 8,137,948 or US Patent Application No. US2011/0229971, the contents of each of which are herein incorporated by reference in their entirety.

### Cell Lysis

Cells of the invention, including, but not limited to viral production cells, may be subjected to cell lysis according to any methods known in the art. Cell lysis may be carried out to obtain one or more agents (e.g. viral particles) present within any cells of the invention. In some embodiments, cell lysis may be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods may be chemical or mechanical. Chemical cell lysis typically comprises contacting one or more cells with one or more lysis agent. Mechanical lysis typically comprises subjecting one or more cells to one or more lysis condition and/or one or more lysis force.

In some embodiments, chemical lysis may be used to lyse cells. As used herein, the term “lysis agent” refers to any agent that may aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents may be selected from lysis salts, amphoteric agents, cationic agents, ionic detergents and non-ionic detergents. Lysis salts may include, but are not limited to sodium chloride (NaCl) and potassium chloride (KCl) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts may be increased or decreased to obtain an effective concentration for rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS), ZWITTERGENT® and the like. Cationic agents may include, but are not limited to cetyltrimethylammonium bromide (C (16) TAB) and Benzalkonium chloride. Lysis agents comprising detergents may include ionic detergents or non-ionic detergents. Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents may include, but are not limited to sodium dodecyl sulfate (SDS), cholate and deoxycholate. In some cases, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100 and Noniodet P-40. Non-ionic detergents are typically weaker lysis agents, but may be included as solubilizing agents for solubilizing cellular and/or viral proteins. Further lysis agents may include enzymes and urea. In some cases, one or more lysis agents may be combined in a lysis solution in order to enhance one or more of cell lysis and protein solubility. In some cases, enzyme inhibitors may be included in lysis solutions in order to prevent proteolysis that may be triggered by cell membrane disruption.

In some embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In some cases, one or more cyroprotectants are included in cell solutions undergoing freeze-thaw lysis. As used herein, the term “cryoprotectant” refers to an agent used to protect one or more substance from damage due to freezing. Cryoprotectants may include any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100. In some cases, cell lysates generated from adherent cell cultures may be treated with one more nuclease to lower the viscosity of the lysates caused by liberated DNA.

In one embodiment, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification

Cell lysates comprising viral particles may be subjected to clarification. Clarification refers to initial steps taken in purification of viral particles from cell lysates. Clarification serves to prepare lysates for further purification by removing larger, insoluble debris. Clarification steps may include, but are not limited to centrifugation and filtration. During clarification, centrifugation may be carried out at low speeds to remove larger debris, only. Similarly, filtration may be carried out using filters with larger pore sizes so that only larger debris is removed. In some cases, tangential flow filtration may be used during clarification. Objectives of viral clarification include high throughput processing of cell lysates and to optimize ultimate viral recovery. Advantages of including a clarification step include scalability for processing of larger volumes of lysate. In some embodiments, clarification may be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and may be carried out according to a variety of available methods including, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In one embodiment, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In some cases, filters useful for clarification of cell lysates may include, but are not limited to ULTIPLEAT PROFILE™ filters (Pall Corporation, Port Washington, N.Y.), SUPOR™ membrane filters (Pall Corporation, Port Washington, N.Y.).

In some cases, flow filtration may be carried out to increase filtration speed and/or effectiveness. In some cases, flow filtration may comprise vacuum filtration. According to such methods, a vacuum is created on the side of the filter opposite that of cell lysate to be filtered. In some cases, cell lysates may be passed through filters by centrifugal forces. In some cases, a pump is used to force cell lysate through clarification filters. Flow rate of cell lysate through one or more filters may be modulated by adjusting one of channel size and/or fluid pressure.

According to some embodiments, cell lysates may be clarified by centrifugation. Centrifugation may be used to pellet insoluble particles in the lysate. During clarification, centrifugation strength (expressed in terms of gravitational units (g), which represents multiples of standard gravitational force) may be lower than in subsequent purification steps. In some cases, centrifugation may be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation may be carried out in order to partition particulates in the cell lysate by sedimentation rate. Gradients used according to methods of the present disclosure may include, but are not limited to cesium chloride gradients and iodixanol step gradients.

### Purification: Chromatography

In some cases, AAV particles may be purified from clarified cell lysates by one or more methods of chromatography. Chromatography refers to any number of methods known in the art for separating out one or more elements from a mixture. Such methods may include, but are not limited to ion exchange chromatography (e.g. cation exchange chromatography and anion exchange chromatography), immunoaffinity chromatography and size-exclusion chromatography. In some embodiments, methods of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

In some embodiments, ion exchange chromatography may be used to isolate viral particles. Ion exchange chromatography is used to bind viral particles based on charge-charge interactions between capsid proteins and charged sites present on a stationary phase, typically a column through which viral preparations (e.g. clarified lysates) are passed. After application of viral preparations, bound viral particles may then be eluted by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. Methods of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, immunoaffinity chromatography may be used. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In some cases, immune compounds may be specific for a particular viral variant. In some cases, immune compounds may bind to multiple viral variants. In some embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8.

In some embodiments, size-exclusion chromatography (SEC) may be used. SEC may comprise the use of a gel to separate particles according to size. In viral particle purification, SEC filtration is sometimes referred to as “polishing.” In some cases, SEC may be carried out to generate a final product that is near-homogenous. Such final products may in some cases be used in pre-clinical studies and/or clinical studies (Kotin, R. M. 2011. Human Molecular Genetics. 20(1):R2-R6, the contents of which are herein incorporated by reference in their entirety.) In some cases, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In one embodiment, the compositions comprising at least one AAV particle may be isolated or purified using the methods described in U.S. Pat. No. 6,146,874, the contents of which are herein incorporated by reference in their entirety.

In one embodiment, the compositions comprising at least one AAV particle may be isolated or purified using the methods described in U.S. Pat. No. 6,660,514, the contents of which are herein incorporated by reference in their entirety.

In one embodiment, the compositions comprising at least one AAV particle may be isolated or purified using the methods described in U.S. Pat. No. 8,283,151, the contents of which are herein incorporated by reference in their entirety.

In one embodiment, the compositions comprising at least one AAV particle may be isolated or purified using the methods described in U.S. Pat. No. 8,524,446, the contents of which are herein incorporated by reference in their entirety.

### Methods of Use

### Gene Silencing: Knockdown Approach

When designed to inhibit or silence a gene, the AAV particles of the present disclosure may also comprise a viral genome that encodes a polynucleotide payload which may be processed to produce an siRNA, miRNA or other double stranded (ds) or single stranded (ss) gene modulatory motif.

Accordingly, the siRNA duplexes or dsRNA can be used to substantially inhibit gene expression in a cell, in particular cells of the CNS. In some aspects, the inhibition of gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The gene can be either a wild type gene or a mutated gene with at least one mutation. Accordingly, the protein is either wild type protein or a mutated polypeptide with at least one mutation.

In some embodiments, the present disclosure provides methods for treating or ameliorating AADC Deficiency (AADCD) and related childhood monoamine neurotransmitter disorders in a subject in need of treatment, the method comprising administering to the subject an effective amount of at least one AAV particle comprising a viral genome encoding an siRNA duplex targeting the abnormal gene and/or protein, delivering duplex into targeted cells, inhibiting the gene expression and protein production, and ameliorating symptoms of the disease or condition in the subject.

### Treatment and Pharmaceutical Compositions

The present disclosure additionally provides a method for treating AADC Deficiency (AADCD) and related childhood monoamine neurotransmitter disorders in a mammalian subject, including a human subject, comprising administering to the subject any of the AAV polynucleotides or AAV genomes described herein (i.e., “viral genomes” or “VGs”) or administering to the subject a particle comprising said AAV polynucleotide or AAV genome, or administering to the subject any of the described compositions, including pharmaceutical compositions.

As used herein the term “composition” comprises an AAV polynucleotide or AAV genome or AAV particle and at least one excipient.

As used herein the term “pharmaceutical composition” comprises an AAV polynucleotide or AAV genome or AAV particle and one or more pharmaceutically acceptable excipients.

Although the descriptions of pharmaceutical compositions, e.g., those AAV particles or viral vectors comprising an AADC-encoding polynucleotide payload e.g., those (including the encoding plasmids or expression vectors, such as viruses, e.g., AAV) comprising a payload, e.g., AADC encoding sequences, to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the AAV particle or viral vector carrying the payload or to the polynucleotide payload delivered by the AAV particle or viral vector as described herein.

In some embodiments, the compositions are administered to pediatric humans. In some embodiments the pediatric humans are between the ages of about 1 month and about 18 years. In some embodiments, the pediatric humans are between the ages of about 1 year and about 12 years. In some embodiments, the pediatric patients are between about 2 years and about 8 years. In some embodiments, the pediatric patients are between about 4 years and about 6 years.

In one embodiment, the compositions described herein are administered to a subject who has been diagnosed with AADC deficiency.

In one embodiment, the compositions described herein are used to decrease motor manifestations of AADC deficiency such as, but not limited to, hypotonia, marked motor delay, and/or involuntary movements such as dystonia and oculogyric crises. The decrease may be for a period of time (e.g., minutes, hours, days, weeks or years) or a reduction in the occurrence of the activity (e.g., a reduction by at least 1%, 5%, 10%, 15%, 20%, 30%, 40%, 50%, 60%, 70%, 80%, 90%, or more than 90%).

In one embodiment, the compositions described herein are used to decrease non-motor manifestations of AADC deficiency such as, but not limited to, autonomic disturbances, emotional liability, cognitive impairment and/or sleep disturbances. The decrease may be for a period of time (e.g., minutes, hours, days, weeks or years) or a reduction in the occurrence of the activity (e.g., a reduction by at least 1%, 5%, 10%, 15%, 20%, 30%, 40%, 50%, 60%, 70%, 80%, 90%, or more than 90%).

In one embodiment, the composition described herein is administered to a subject who has received dopaminergic therapy. As a non-limiting example, the subject may have a limited response to dopaminergic therapy.

In one embodiment, the composition of the present invention is used as described by Hwu and colleagues (Hwu, W. L., et al., 2012. Gene therapy for aromatic L-amino acid decarboxylase deficiency. Sci. Transl. Med. Vol. 4, 134ra61, the contents of which are herein incorporated by reference in their entirety), wherein four AADCD patients were treated with parenchymal delivery to the putamen of a recombinant AAV2 carrying human AADC. Components of the AAV2-hAADC vector included a cytomegalovirus immediate early gene promoter, the first intron of human growth factor, human AADC complementary DNA (cDNA), and a simian virus 40 polyadenylation signal. Each of the patients had mutations in the DDC gene (IVS6+4 A>T, or the less common c. 1927_1298insA), known to cause AADCD. Prior to treatment, patients were bed-ridden and displayed little to no spontaneous movement. After treatment, patients showed increased motor and cognitive performance as evaluated by the Alberta Infant Motor Scale (AIMS), Peabody Developmental Motor Scale (PDMS-II) and the Comprehensive Developmental Inventory for Infants and Toddlers (CDIIT). At 16 months subsequent to treatment, one patient was able to stand with assistance. Patients also showed improvement in non-motor symptoms, such as the occurrence of oculogyric crises, increased emotional stability and improvements in sweating and hypothermic conditions. Evaluations of neurotransmitters and their metabolites by PET and HPLC demonstrated increased dopamine, serotonin, homovanillic acid (HVA) and 5-hydroxyindoleactic acid (HIAA). Disadvantages to this approach included limited transduction of the vector to a small region of the putamen and increased anti-AAV2 neutralizing antibodies following treatment. In a non-limiting example, the viral particle of the present invention is an AAV2-CMV-hGH intron-hAADC-SV40 poly(A) vector. In a non-limiting example, use of the AAV particles of the present invention result in increased scores on the AIMS, PDMS-II and CDIIT evaluations. In another non-limiting example, administration of the AAV particles of the present invention results in improved motor and cognitive behavior in AADCD patients. In one embodiment, administration of the AAV particle of the present invention, leads to improved scores on AIMS, PDMS-II and/or CDIIT. In a non-limiting example the score increases 10 points. In another non-limiting example, the score increases 1 point. In another non-limiting example, the score increases 1, 2, 3, 4, 5, 6, 7, 8, 9, 10, 11, 12, 13, 14, 15, 16, 17, 18, 19, or 20 points. In another non-limiting example, use of the AAV particles of the present invention results in increased dopamine and serotonin levels.

In one embodiment, the AAV particle of the present invention may be or is as described in United States Publication No. US20120220648, the contents of which are herein incorporated by reference in their entirety. The AAV2-hAADC of SEQ ID NO: 1 of US20120220648 comprises a cytomegalovirus immediate early gene promoter, a P globin intron, a human AADC polynucleotide, and a simian virus 40 polyadenylation signal between two AAV2 ITRs with kanamycin resistance. As a non-limiting example, the AAV particle of the present invention may comprise any part of SEQ ID NO: 1 of US20120220648 or a variant thereof.

In one embodiment, the AAV particle of the present invention may be used in a compassionate treatment or clinical trial. As a non-limiting example of a compassionate treatment trial, Hwu and colleagues treated 8 patients with AADCD with AAV2-hAADC gene therapy. Treatment improved motor performance in all patients (4 able to stand with support, 3 able to sit with support), with the longest follow up exceeding four years following treatment. Further, no virus-associated toxicity has been identified. An ongoing Phase I/II clinical trial (NCT01395641, the contents of which are herein incorporated by reference in their entirety) by the same team is being used to assess the safety and efficacy of AAV2-hAADC treatment in patients with AADCD. Stereotactic surgery will be used to inject AAV2-hAADC bilaterally into the putamen (intracerebral infusion) of AADCD patients. Primary outcomes include increases in CSF levels of neurotransmitter metabolites HVA or HVIAA at a year post-treatment as well as an increase of 10 points or more on the PDMS-II. Secondary outcomes include safety measures of post-surgery intracerebral hemorrhage, post-surgery CSF leakage and severity of post-gene therapy dyskinesia and efficacy end points of body weight gain, increased signal in the putamen as measured by DOPA-PET (signal of de novo dopamine production), and increased score in other motor or developmental evaluations. The Clinical Trials registry indicates an estimated enrollment of 10 patients with a study completion date of September 2016.

In one embodiment, the AAV particle of the present invention is and is used to treat AADCD as described in Clinical Trial No. NCT01395641, the contents of which are herein incorporated by reference in their entirety.

In one embodiment, the AAV particle of the present invention is an AAV-hAADC, also known as AGIL-AADC, as described by Agilis Biotherapeutics.

In one embodiment, the AAV particle of the present invention is an AAV-hAADC, also known as AGIL-AADC, as described by Agilis Biotherapeutics and is the same as used in clinical trials by Hwu and colleagues, described herein.

The AAV particles of the present invention may be used or tested in any AADC animal model, including, but not limited to the Ddc^{KI} mouse as described in Lee, N.C., et al 2013 Regulation of the dopaminergic system in a murine model of aromatic L-amino acid decarboxylase deficiency. Neurobiol. Dis. 52:177-190; Lee, N.C., et al., 2015 Benefits of neuronal preferential systemic gene therapy for neurotransmitter deficiency. Mol. Ther. Vol. 23(10), 1572-1581; Lee, N.C., et al., 2013 Treatment of congenital neurotransmitter deficiencies by intracerebral ventricular injection of an adeno-associated virus serotype 9 vector. Hum. Gen. Ther. 25:189-198; and Hwu, W. L., et al., 2013 AADC deficiency: occurring in humans, modeled in rodents. Adv Pharmacol 68:273-284, the contents of each of which are herein incorporated by reference in their entirety. Non-limiting examples of animal models of AADC deficiency include mouse, non-human primate, rat, ferret, zebrafish, drosophila or c. elegans.

Based on the IVS6+4A>T mutation known to cause AADCD, a mouse model was created (Ddc^{KI} mice), in which a splicing mutation was knocked into the Ddc gene, along with a neomycin-resistance gene, to generate exon 6 skipping in the mRNA (Lee, N.C., et a 2013 Regulation of the dopaminergic system in a murine model of aromatic L-amino acid decarboxylase deficiency. Neurobiol. Dis. 52:177-190; Lee, N.C., et al., 2015 Benefits of neuronal preferential systemic gene therapy for neurotransmitter deficiency. Mol. Ther. Vol. 23(10), 1572-1581; Lee, N.C., et al., 2013 Treatment of congenital neurotransmitter deficiencies by intracerebral ventricular injection of an adeno-associated virus serotype 9 vector. Hum. Gen. Ther. 25:189-198; and Hwu, W. L., et al., 2013 AADC deficiency: occurring in humans, modeled in rodents. Adv Pharmacol 68:273-284, the contents of each of which are herein incorporated by reference in their entirety). Mouse Ddc knockout causes embryonic lethality, making this strategy not viable as an option for design of a mouse model. Meanwhile, the human IVS6+4A>T DDC gene mutation is known to interrupt the consensus sequence in intron 6, thereby causing inappropriate splicing. Homozygous knock-in mice survive into adulthood and are phenotypically similar to the clinical presentation of AADCD. Ddc^{KI} mice demonstrate disturbed weight gain, decreased survival and numerous behavioral, motor and autonomic dysfunctions. Interestingly, these mice show signs of spontaneous recovery with age, introducing a distinct caveat into this animal model.

Treatment of neonatal (P0) Ddc^{KI} mice by intracerebroventricular injection of AAV9-hAADC yielded improvements in weight gain, survival, dopamine and serotonin levels, cardiovascular function and motor behavior. The AAV9-AADC comprised a ubiquitous cytomegalovirus immediate early gene promoter, the first intron of human growth hormone, human AADC cDNA (NM_000790), a simian virus 40 polyadenylation signal, AAV2 ITRs and an AAV9 capsid. The AAV9-AADC vector was delivered by bilateral infusions of 2×10¹⁰ vg in 2 μL to the lateral ventricles using a Hamilton syringe. Despite some improvements in the Ddc^{KI} mice, delivery of the AAV9-AADC resulted in a hyperactivity, thereby negating some of the therapeutic benefit. (see Lee, N.C., et al., 2013 Treatment of congenital neurotransmitter deficiencies by intracerebral ventricular injection of an adeno-associated virus serotype 9 vector. Hum. Gen. Ther. 25:189-198, the contents of which are herein incorporated by reference in their entirety).

Translation of neonatal ICV injections to larger animals or human patients is challenging given the time frame of the development of the cranial bones and the blood brain barrier. In a follow-up study, treatment of young 7 day old Ddc^{M}K mice by intraperitoneal injection (systemic delivery) with AAV-AADC resulted in improvements in weight gain, survival, dopamine and serotonin levels, cardiovascular function, and motor and behavioral function. Two AAV-AADC vectors were tested, one that targeted neurons specifically and the other, previously described AAV9-AADC, which showed more ubiquitous targeting. Of the two vectors, the neuronal targeting vector showed superior efficacy in treating the Ddc^{KI} mice. The neuronal targeting AAV-AADC (AAVN-AADC) comprised a synapsin-I promoter for neuronal targeting, mouse AADC (NM_016672.4), a woodchuck hepatitis virus post-transcriptional regulatory element, AAV3 ITRs and an AAV9 capsid with a tyrosine to phenylalanine substitution at position 446 of the AAV9 capsid sequence.

In one embodiment, the AAV particle of the present invention is an AAVN-AADC, comprising a synapsin-I promoter, an AADC polynucleotide, a woodchuck hepatitis virus post-transcriptional regulatory element, AAV3 ITRs and an AAV9 capsid as described in Lee, N.C., et al., 2015 Benefits of neuronal preferential systemic gene therapy for neurotransmitter deficiency. Mol. Ther. Vol. 23(10), 1572-1581, the contents of which are herein incorporated by reference in their entirety. In one embodiment the AADC polynucleotide is a mouse AADC. In another embodiment the AADC polynucleotide is a human AADC.

In one embodiment, the AAV particle of the present invention is an AAV9-AADC, comprising a ubiquitous CMV promoter, the first intron of human growth hormone, an AADC polynucleotide, an SV40 poly(A), AAV2 ITRs and an AAV9 capsid, as described in Lee, N.C., et al., 2015 Benefits of neuronal preferential systemic gene therapy for neurotransmitter deficiency. Mol. Ther. Vol. 23(10), 1572-1581, the contents of which are herein incorporated by reference in their entirety. In one embodiment the AADC polynucleotide is a mouse AADC. In another embodiment the AADC polynucleotide is a human AADC.

The AAV particle of the present invention may be an AAV2-hAADC, AAVN-AADC, or AAV9-AADC as described in United States Publication No. US20120220648; Hwu, W. L., et al., 2012 Gene therapy for aromatic L-amino acid decarboxylase deficiency. Sci. Transl. Med. Vol. 4, 134ra61; Lee, N.C., et al., 2013 Treatment of congenital neurotransmitter deficiencies by intracerebral ventricular injection of an adeno-associated virus serotype 9 vector. Hum. Gen. Ther. 25:189-198; and Lee, N.C., et al., 2015 Benefits of neuronal preferential systemic gene therapy for neurotransmitter deficiency. Mol. Ther. Vol. 23(10), 1572-1581, the contents of each of which are herein incorporated by reference in their entirety.

### Circadian Rhythm and Sleep-Wake Cycles

Circadian rhythms are physical, mental and behavioral changes that tend to follow a 24 hour cycle. Circadian rhythms can influence sleep-wake cycles, hormone release, body temperature and other bodily functions. Changes in the circadian rhythm can cause conditions and/or disorder such as, but not limited to sleep disorders (e.g., insomnia), depression, bipolar disorder, seasonal affective disorder, obesity and diabetes.

In one embodiment, the AADC polynucleotides described herein may be used to treat insomnia.

The sleep-wake cycle comprises periods of sleep and periods of wake. Generally, in a 24 hours period the total hours of sleep are less than the total hours of wakefulness. As a non-limiting example, the sleep-wake cycle comprises 7-9 hours of sleep and 15-17 hours of wakefulness. As a non-limiting example, the sleep-wake cycle comprises 8 hours of sleep and 16 hours of wakefulness. As a non-limiting example, the sleep-wake cycle comprises 8-10 hours of sleep and 14-16 hours of wakefulness.

In one embodiment, the sleep-wake cycle of a subject is improved by administration to the subject of the AADC polynucleotides described herein.

In one embodiment, the sleep-wake cycle of a subject is regulated by administration to the subject of the AADC polynucleotides described herein. As a non-limiting example, the regulation may be the correction of more periods of sleep occurring at night and less periods of sleep occurring during the day.

In one embodiment, the sleep-wake cycle of a subject administered the AADC polynucleotides described herein improves as compared to the sleep-wake cycle of the subject prior to administration of the AADC polynucleotides. As a non-limiting example, the subject has an increased period of sleep and a decreased period of wakefulness. As another non-limiting example, the subject has a decreased period of sleep and an increased period of wakefulness.

In one embodiment, the sleep-wake cycle of a subject administered the AADC polynucleotides described herein is regulated as compared to the sleep-wake cycle of the subject prior to administration of the AADC polynucleotides. As a non-limiting example, the length of the periods of sleep and the periods of wakefulness may be about the same (e.g., +/−1 hour) for at least 2 days. As another non-limiting example, the length of the periods of sleep and the periods of wakefulness if a 24 hours period may be within 10 minutes, 15 minutes, 20 minutes, 25 minutes, 30 minutes, 35 minutes, 40 minutes, 45 minutes, 50 minutes, 55 minutes, 1 hour, 1.5 hours, or 2 hours of the previous 24 hour period.

In one embodiment, the amount of rapid eye movement (REM) sleep a subject experiences in a 24 hour period is altered after the subject is administered the AADC polynucleotides described herein. REM sleep is generally considered an active period of sleep marked by intense brain activity where brain waves are fast and desynchronized. An adult, on average, spends about 20-25% of their total daily sleep period in REM sleep. As a non-limiting example, the amount of REM sleep is decreased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of REM sleep is decreased by 1-1⁰%, 5-10%, 5-15%, 10-15%, 15-20%, 15-25%, 20-25%, 20-30%, 25-30%, 25-35%, 30-35%, 30-40%, 35-40%, 40-50% or 40-60%. As a non-limiting example, the amount of REM sleep is increased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of REM sleep is increased by 1-5%, 1-10%, 5-10%, 5-15%, 10-15%, 15-20%, 15-25%, 20-25%, 20-30%, 25-30%, 25-35%, 30-35%, 30-40%, 35-40%, 40-50% or 40-60%.

In one embodiment, the amount of non-REM (NREM) sleep a subject experiences in a 24 hour period is altered after the subject is administered the AADC polynucleotides described herein. NREM sleep is generally characterized by a reduction in physiological activity since the brain waves, as measured by EEG, get slower and have greater amplitude. NREM has four stages: Stage 1 is the time of drowsiness or transition from being awake to falling asleep where the brain waves and muscle activity begin to slow; Stage 2 is a period of light sleep during which eye movements stop and brain waves become slower with occasional bursts of rapid waves (sometimes called sleep spindles); Stage 3 and Stage 4 (collectively referred to as slow wave sleep) are characterized by the presence of slow brain waves (delta waves) interspersed with smaller faster waves where there are no eye movements. An adult, on average, spends about 75-80% of their total daily sleep period in NREM sleep with about half of their total daily sleep time in NREM stage 2 sleep.

In one embodiment, the amount of NREM sleep a subject experiences is altered after the subject is administered the AADC polynucleotides described herein. As a non-limiting example, the amount of NREM sleep is decreased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM sleep is decreased by 1-10%, 5-10%, 5-15%, 10-15%, 15-20%, 15-25%, 20-25%, 20-30%, 25-30%, 25-35%, 30-35%, 30-40%, 35-40%, 40-50% or 40-60%. As a non-limiting example, the amount of NREM sleep is increased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM sleep is increased by 1-5%, 1-10%, 5-10%, 5-15%, 10-15%, 15-20%, 15-25%, 20-25%, 20-30%, 25-30%, 25-35%, 30-35%, 30-40%, 35-40%, 40-50% or 40-60%.

In one embodiment, the amount of NREM Stage 1 sleep a subject experiences is altered after the subject is administered the AADC polynucleotides described herein. As a non-limiting example, the amount of NREM Stage 1 sleep is decreased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 1 sleep is decreased by 1-10%, 5-10%, 5-15%, 10-15%, 15-20%, 15-25%, 20-25%, 20-30%, 25-30%, 25-35%, 30-35%, 30-40%, 35-40%, 40-50% or 40-60%. As a non-limiting example, the amount of NREM Stage 1 sleep is increased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 1 sleep is increased by 1-5%, 1-10%, 5-10%, 5-15%, 10-15%, 15-20%, 15-25%, 20-25%, 20-30%, 25-30%, 25-35%, 30-35%, 30-40%, 35-40%, 40-50% or 40-60%.

In one embodiment, the amount of NREM Stage 2 sleep a subject experiences is altered after the subject is administered the AADC polynucleotides described herein. As a non-limiting example, the amount of NREM Stage 2 sleep is decreased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 2 sleep is decreased by 1-10%, 5-10%, 5-15%, 10-15%, 15-20%, 15-25%, 20-25%, 20-30%, 25-30%, 25-35%, 30-35%, 30-40%, 35-40%, 40-50% or 40-60%. As a non-limiting example, the amount of NREM Stage 2 sleep is increased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 2 sleep is increased by 1-5%, 1-10%, 5-10%, 5-15%, 10-15%, 15-20%, 15-25%, 20-25%, 20-30%, 25-30%, 25-35%, 30-35%, 30-40%, 35-40%, 40-50% or 40-60%.

In one embodiment, the amount of NREM Stage 3 and 4 sleep a subject experiences is altered after the subject is administered the AADC polynucleotides described herein. As a non-limiting example, the amount of NREM Stage 3 and 4 sleep is decreased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 3 and 4 sleep is decreased by 1-10%, 5-10%, 5-15%, 10-15%, 15-20%, 15-25%, 20-25%, 20-30%, 25-30%, 25-35%, 30-35%, 30-40%, 35-40%, 40-50% or 40-60%. As a non-limiting example, the amount of NREM Stage 3 and 4 sleep is increased by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65% or more than 65%. As a non-limiting example, the amount of NREM Stage 3 and 4 sleep is increased by 1-5%, 1-10%, 5-10%, 5-15%, 10-15%, 15-20%, 15-25%, 20-25%, 20-30%, 25-30%, 25-35%, 30-35%, 30-40%, 35-40%, 40-50% or 40-60%.

In one embodiment, periods of NREM and REM cycles are more consistent in a subject after the subject is administered the AADC polynucleotides described herein. Generally NREM and REM cycles alternate every 90 to 110 minutes four to six times per night.

### Formulation and Delivery

### Formulation

The AAV particles or viral vectors can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. AAV), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In one embodiment, the pharmaceutical composition comprises a recombinant adeno-associated virus (AAV) vector comprising an AAV capsid and an AAV vector genome. The AAV vector genome may comprise at least one AADC polynucleotide described herein, such as, but not limited to, SEQ ID NOs 6, 7, 8, 9, 17, 18, 19, 20, 21, 22, and 23 or variants having at least 95% identity thereto. The recombinant AAV vectors in the pharmaceutical composition may have at least 70% which contain an AAV vector genome.

In some embodiments, the AAV formulations described herein may contain at least one payload molecule, e.g., an AADC polynucleotide. As a non-limiting example, the formulations may contain 1, 2, 3, 4 or 5 AADC polynucleotide payload molecules. In one embodiment the formulation may contain a payload encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three AAV payload encoding proteins.

The formulations can include one or more excipients, each in an amount that together increases the stability of the AAV particle, increases cell transfection or transduction by the viral particle, increases the expression of viral polynucleotide encoded protein, and/or alters the release profile of AAV polynucleotide encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In one embodiment, the AADC polynucleotides may be formulated in a hydrogel prior to administration. Hydrogels have a degree of flexibility which is similar to natural tissue as a result of their significant water content.

In another embodiment, a hydrogel may be administered to a subject prior to the administration of an AADC polynucleotide formulation. As a non-limiting example, the site of administration of the hydrogel may be within 3 inches (e.g., within 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 or less than 0.1 inches) of the site of administration of the AADC polynucleotide formulation.

In one embodiment, the AADC polynucleotides may be administered in saline. As a non-limiting example, the formulation may be phosphate buffered saline (PBS) with 0.001% Pluronic acid (F-68). Additionally the formulation may be sterilized.

### Inactive Ingredients

In some embodiments, AADC polynucleotide formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of AAV particles and viral vectors carrying AADC polynucleotides disclosed herein may include cations or anions. In one embodiment, the formulations include metal cations such as, but not limited to, Zn2+, Ca2+, Cu2+, Mg+ and combinations thereof. As a non-limiting example, formulations may include polymers and AADC polynucleotides complexed with a metal cation (See e.g., U.S. Pat. Nos. 6,265,389 and 6,555,525, each of which is herein incorporated by reference in its entirety).

### Administration

The AAV particles and viral vectors comprising AADC polynucleotides of the present disclosure may be administered by any route which results in a therapeutically effective outcome. These include, but are not limited to enteral (into the intestine), gastroenteral, epidural (into the dura matter), intracerebral (into the cerebrum), intracerebroventricular (into the cerebral ventricles, a.k.a. “intraventricular”), intradural (within or beneath the dura), intrastriatal (within the striatum, caudate nucleus and/or putamen), peridural, epicutaneous (application onto the skin), subcutaneous (under the skin), intradermal (into the skin itself), transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), nasal administration (through the nose), intravenous (into a vein), intravenous bolus, intravenous drip, intraarterial (into an artery), intramuscular (into a muscle), intracardiac (into the heart), intraosseous infusion (into the bone marrow), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oral (by way of the mouth), oropharyngeal (directly to the mouth and pharynx), parenteral, percutaneous, periarticular, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier (BBB), vascular barrier, or other epithelial barrier. In some embodiments, compositions may be administered to the substantia nigra, in particular, to the substantia nigra pars compacta (SNpc), and to the ventral tegmental area (VTA). In one embodiment, a formulation for a route of administration may include at least one inactive ingredient.

In one embodiment, the viral vectors comprising AADC polynucleotides of the present invention may be administered to the ventricles.

In one embodiment, the viral vectors comprising AADC polynucleotides of the present invention may be administered to the ventricles by intracerebroventricular delivery.

In one embodiment, the viral vectors comprising AADC polynucleotides of the present invention may be administered systemically.

In one embodiment, the viral vectors comprising AADC polynucleotides of the present invention may be administered systemically by intraperitoneal injection.

In one embodiment, the viral vectors comprising AADC polynucleotides of the present invention may be administered systemically by intravascular injection.

In one embodiment, the viral vectors comprising AADC polynucleotides of the present invention may be administered to the putamen.

In one embodiment, the viral vectors comprising AADC polynucleotides of the present invention may be administered to the right putamen and/or the left putamen. The administration may be at one or more sites in the putamen such as, but not limited to, 2 sites, 3 sites, 4 sites or more than 4 sites. As a non-limiting example, the viral vectors comprising AADC polynucleotides of the present invention are delivered to 2 sites in the left putamen and 2 sites in the right putamen.

In one embodiment, the administration of the formulation of the viral vectors comprising the AADC polynucleotides of the present invention to a subject provides coverage of the putamen of a subject (e.g., the left and/or right putamen). In one aspect, the administration of the viral vectors comprising the AADC polynucleotides may provide at least 8%, 9%, 10%, 13%, 14%15%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% to the left and/or right putamen of a subject. As a non-limiting example, the coverage is at least 20%. As a non-limiting example, the coverage is at least 40%. In another aspect, the administration of the viral vectors comprising the AADC polynucleotides may provide at least 8%, 9%, 10%, 13%, 14%, 15%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% coverage of the surface area of the left and/or right putamen of a subject. As a non-limiting example, the total coverage is at least 20%. As a non-limiting example, the total coverage is at least 40%. In yet another aspect, the administration of the viral vectors comprising the AADC polynucleotides may provide 10-40%, 20-40%, 20-30%, 20-35%, 20-50%, 30-40%, 35-40%, 30-60%, 40-70%, 50-80% or 60-90% coverage to the left and/or right putamen of a subject or to the total surface area of the left and/or right putamen of a subject.

In one embodiment, the administration of the formulation of the viral vectors comprising the AADC polynucleotides of the present invention to a subject provides coverage of the posterior putamen of a subject (e.g., the left and/or right posterior putamen). In one aspect, the administration of the viral vectors comprising the AADC polynucleotides may provide at least 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% to the left and/or right posterior putamen of a subject. As a non-limiting example, the coverage is at least 20%. As a non-limiting example, the coverage is at least 40%. In another aspect, the administration of the viral vectors comprising the AADC polynucleotides may provide at least 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 60%, 65%, 70%, 75%, 80%, 85%, 90%, 95% or more than 95% coverage of the surface area of the left and/or right posterior putamen of a subject. As a non-limiting example, the total coverage is at least 20%. As a non-limiting example, the total coverage is at least 40%. In yet another aspect, the administration of the viral vectors comprising the AADC polynucleotides may provide 10-40%, 20-50%, 30-60%, 40-70%, 50-80% or 60-90% coverage to the left and/or right posterior putamen of a subject or to the total surface area of the left and/or right putamen of a subject.

In one embodiment, a subject may be administered the viral-vectors comprising AADC polynucleotides of the present invention safely delivered to substantia nigra pars compacta (SNpc) and ventral tegmental area (VTA) via bilateral infusions, or alternatively, intrastriatally (into the caudate nucleus and putamen), or into the subthalamic nucleus (STN).

In one embodiment, the AADC polynucleotides described herein may be administered bilaterally to the putamen.

In one embodiment, the AADC polynucleotides described herein may be administered bilaterally to the putamen by parenchymal delivery.

In one embodiment, the AADC polynucleotides described herein may be administered using acute bilateral placement of catheters into each putamen. The placement may use magnetic resonance image (MRI)-guided stereotactic neurosurgical techniques known in the art or described herein. Additionally, a contrast agent such as, but not limited to a gadolinium based contrast agent (e.g., PROHANCE®) may be used in the formulation to monitor and confirm the distribution of the formulation.

In one embodiment, a subject may be administered the viral vectors comprising AADC polynucleotides of the present invention in a bilateral stereotactic CED-assisted step infusion into the putamen (e.g., the post commissural putamen).

The AAV particle of the present invention may be administered or delivered to a subject suffering from AADC deficiency by any of the methods described in United States Publication No. US20120220648; Clinical Trial NCT01395641; Hwu, W. L., et al., 2012 Gene therapy for aromatic L-amino acid decarboxylase deficiency. Sci. Transl. Med. Vol. 4, 134ra61; Lee, N.C., et al., 2013 Treatment of congenital neurotransmitter deficiencies by intracerebral ventricular injection of an adeno-associated virus serotype 9 vector. Hum. Gen. Ther. 25:189-198; and Lee, N.C., et al., 2015 Benefits of neuronal preferential systemic gene therapy for neurotransmitter deficiency. Mol. Ther. Vol. 23(10), 1572-1581, the contents of each of which are herein incorporated by reference in their entirety.

In one embodiment, the AAV particle of the present invention is delivered by intraperitoneal injection (systemic delivery) as described in Lee, N.C., et al., 2015 Benefits of neuronal preferential systemic gene therapy for neurotransmitter deficiency. Mol. Ther. Vol. 23(10), 1572-1581, the contents of which are herein incorporated by reference in their entirety.

In one embodiment, delivery of viral vector pharmaceutical compositions in accordance with the present invention to cells of the central nervous system (e.g., parenchyma) comprises a rate of delivery defined by VG/hour=mL/hour*VG/mL, wherein VG is viral genomes, VG/mL is composition concentration, and mL/hour is rate of prolonged infusion.

In one embodiment, delivery of viral vector pharmaceutical compositions in accordance with the present invention to cells of the central nervous system (e.g., parenchyma) comprises infusion of up to 1 mL. In one embodiment, delivery of viral vector pharmaceutical compositions in accordance with the present invention to cells of the central nervous system (e.g., parenchyma) may comprise infusion of 0.0001, 0.0002, 0.001, 0.002, 0.003, 0.004, 0.005, 0.008, 0.010, 0.015, 0.020, 0.025, 0.030, 0.040, 0.050, 0.060, 0.070, 0.080, 0.090, 0.1, 0.2, 0.3, 0.4, 0.5, 0.6, 0.7, 0.8, or 0.9 mL. In a non-limiting example the AAV particles of the present invention are infused in a volume of 80 L. In another non-limiting example the AAV particles of the present invention are infused in a volume of 20 μL. In a non-limiting example, the AAV particles of the present invention are infused in a volume of 2 μL.

In one embodiment, delivery of viral vector pharmaceutical compositions in accordance with the present invention to cells of the central nervous system (e.g., parenchyma) comprises infusion of between about 1 mL to about 120 mL. In one embodiment, delivery of viral vector pharmaceutical compositions in accordance with the present invention to cells of the central nervous system (e.g., parenchyma) may comprise an infusion of 0.1, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In one embodiment, delivery of AAV particles to cells of the central nervous system (e.g., parenchyma) comprises infusion of at least 3 mL. In one embodiment, delivery of AAV particles to cells of the central nervous system (e.g., parenchyma) consists of infusion of 3 mL. In one embodiment, delivery of AAV particles to cells of the central nervous system (e.g., parenchyma) comprises infusion of at least 10 mL. In one embodiment, delivery of AAV particles to cells of the central nervous system (e.g., parenchyma) consists of infusion of 10 mL.

In one embodiment, the volume of the viral vector pharmaceutical composition delivered to the cells of the central nervous system (e.g., parenchyma) of a subject is 2 ul, 20 ul, 50 ul, 80 ul, 100 ul, 200 ul, 300 ul, 400 ul, 500 ul, 600 ul, 700 ul, 800 ul, 900 ul, 1000 ul, 1100 ul, 1200 ul, 1300 ul, 1400 ul, 1500 ul, 1600 ul, 1700 ul, 1800 ul, 1900 ul, 2000 ul or more than 2000 ul. In a non-limiting example the AAV particles of the present invention are infused in a volume of 80 ul. In another non-limiting example the AAV particles of the present invention are infused in a volume of 20 ul. In a non-limiting example, the AAV particles of the present invention are infused in a volume of 2 ul.

In one embodiment, the volume of the viral vector pharmaceutical composition delivered to a region in both hemispheres of a subject brain is 2 ul, 20 ul, 50 ul, 80 ul, 100 ul, 200 ul, 300 ul, 400 ul, 500 ul, 600 ul, 700 ul, 800 ul, 900 ul, 1000 ul, 1100 ul, 1200 ul, 1300 ul, 1400 ul, 1500 ul, 1600 ul, 1700 ul, 1800 ul, 1900 ul, 2000 ul or more than 2000 ul. As a non-limiting example, the volume delivered to a region in both hemispheres is 200 ul. As another non-limiting example, the volume delivered to a region in both hemispheres is 900 ul. As yet another non-limiting example, the volume delivered to a region in both hemispheres is 1800 ul.

In one embodiment, the volume of the viral vector pharmaceutical composition delivered to the putamen in both hemispheres of a subject brain is 2 ul, 20 ul, 50 ul, 80 ul, 100 ul, 200 ul, 300 ul, 400 ul, 450 ul, 500 ul, 600 ul, 700 ul, 800 ul, 900 ul, 1000 ul, 1100 ul, 1200 ul, 1300 ul, 1400 ul, 1500 ul, 1600 ul, 1700 ul, 1800 ul, 1900 ul, 2000 ul or more than 2000 ul. As a non-limiting example, the volume delivered to the putamen in both hemispheres is 100 ul. As another non-limiting example, the volume delivered to the putamen in both hemispheres is 200 ul. As a non-limiting example, the volume delivered to the putamen in both hemispheres is 300 ul. As another non-limiting example, the volume delivered to the putamen in both hemispheres is 450 ul. As another non-limiting example, the volume delivered to the putamen in both hemispheres is 900 ul. As yet another non-limiting example, the volume delivered to the putamen in both hemispheres is 1800 ul.

In one embodiment, the total volume delivered to a subject may be split between one or more administration sites e.g., 1, 2, 3, 4, 5 or more than 5 sites. As a non-limiting example, the total volume is split between administration to the left and right putamen. As another non-limiting example, the total volume is split between two sites of administration to each of the left and right putamen.

In one embodiment, the viral vector pharmaceutical composition is administered using a fenestrated needle. Non-limiting examples of fenestrated needles are described in U.S. Pat. Nos. 8,333,734, 7,135,010, 7,575,572, 7,699,852, 4,411,657, 6,890,319, 6,613,026, 6,726,659, 6,565,572, 6,520,949, 6,382,212, 5,848,996, 5,759,179, 5,674,267, 5,588,960, 5,484,401, 5,199,441, 5,012,818, 4,474,569, 3,766,907, 3,552,394, the contents of each of which are herein incorporated by reference in their entirety.

In one embodiment, a composition comprises AADC polynucleotides described herein and the AADC polynucleotides are components of an AAV viral genome packaged in an AAV viral particle. The percent (%) ratio of AAV viral particles comprising the AADC polynucleotide (also referred to herein and AADC particles) to the AAV viral particles without the AADC polynucleotide (also referred to herein as empty capsids) in the composition may be 0:100, 1:99, 0:90, 15:85, 25:75, 30:70, 50:50, 70:30, 85:15, 90:10, 99:1 or 100:0. As a non-limiting example, the percent ratio of AADC particles to empty capsids is 50:50. As another non-limiting example, the percent ratio of AADC particles to empty capsids is 70:30. As another non-limiting example, the percent ratio of AADC particles to empty capsids is 85:15. As another non-limiting example, the percent ratio of AADC particles to empty capsids is 100:0.

In one embodiment, the composition described herein comprises at least 1, 5, 10, 15, 20, 25, 30, 35, 40, 45, 50, 51, 52, 53, 54, 55, 60, 65, 70, 75, 80, 81, 82, 83, 84, 85, 86, 87, 88, 89, 90, 91, 92, 93, 94, 95, 96, 97, 98, 99 or greater than 99% AADC particles. As a non-limiting example, the composition comprises at least 50% AADC particles. As another non-limiting example, the composition comprises at least 52% AADC particles. As another non-limiting example, the composition comprises at least 58% AADC particles. As another non-limiting example, the composition comprises at least 70% AADC particles. As another non-limiting example, the composition comprises at least 83% AADC particles. As another non-limiting example, the composition comprises at least 85% AADC particles. As another non-limiting example, the composition comprises at least 99% AADC particles. As another non-limiting example, the composition comprises 100% AADC particles.

In one embodiment, the composition described herein comprises 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% AADC particles. As a non-limiting example, the composition described herein comprises 50-100% AADC particles. As another non-limiting example, the composition described herein comprises 50-60% AADC particles. As another non-limiting example, the composition described herein comprises 80-99% AADC particles. As another non-limiting example, the composition described herein comprises 80-90% AADC particles. As a non-limiting example, the composition described herein comprises 80-95% AADC particles. As a non-limiting example, the composition described herein comprises 80-85% AADC particles.

In one embodiment, the composition described herein comprises less than 1, 5, 10, 15, 20, 25, 30, 35, 40, 45, 50, 51, 52, 53, 54, 55, 60, 65, 70, 75, 80, 81, 82, 83, 84, 85, 86, 87, 88, 89, 90, 91, 92, 93, 94, 95, 96, 97, 98, 99 or 100% empty particles. As a non-limiting example, the composition comprises less than 50% empty particles. As a non-limiting example, the composition comprises less than 45% empty particles. As a non-limiting example, the composition comprises less than 40% empty particles. As a non-limiting example, the composition comprises less than 35% empty particles. As a non-limiting example, the composition comprises less than 30% empty particles. As a non-limiting example, the composition comprises less than 25% empty particles. As a non-limiting example, the composition comprises less than 20% empty particles. As a non-limiting example, the composition comprises less than 15% empty particles. As a non-limiting example, the composition comprises less than 10% empty particles. As a non-limiting example, the composition comprises less than 5% empty particles. As a non-limiting example, the composition comprises less than 1% empty particles.

In one embodiment, the composition described herein comprises 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% empty particles. As a non-limiting example, the composition described herein comprises 30-40% empty particles. As another non-limiting example, the composition described herein comprises 30-50% AADC particles. As another non-limiting example, the composition described herein comprises 30-60% empty particles. As another non-limiting example, the composition described herein comprises 30-70% empty particles. As a non-limiting example, the composition described herein comprises 30-80% empty particles. As a non-limiting example, the composition described herein comprises 30-90% empty particles.

### Dosing

The present disclosure provides methods comprising administering AAV particles or viral vectors and their payload or complexes in accordance with the disclosure to a subject in need thereof. AAV particle or viral vector pharmaceutical, imaging, diagnostic, or prophylactic compositions may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific payload employed; the duration of the treatment; drugs used in combination or coincidental with the specific payload employed; and like factors well known in the medical arts.

In certain embodiments, AAV particle or viral vector pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to vg or viral genomes per kg or into total viral genomes administered by one of skill in the art.

In certain embodiments, AAV particle or viral vector pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site.

The desired dosage may be delivered three times in a single day, two times in a single day, once in a since day or in a period of 24 hours the dosage may be delivered once, twice, three times or more than three times. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the AAV particles or viral vectors of the present disclosure are administered to a subject in split doses, and may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In one embodiment, delivery of viral vector pharmaceutical compositions in accordance with the present invention to cells of the central nervous system (e.g., parenchyma) may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10, 3×10⁸, 4×10⁸, 5×10, 6×10, 7×10, 8×10, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 1.8×10¹¹, 2×10¹¹, 3×10¹¹, 4×10¹¹, 5×10¹¹, 5.5×10¹¹, 6×10¹¹, 7×10¹¹, 8×10¹¹, 9×10¹¹, 0.8×10¹², 0.83×10¹², 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.3×10¹³, 3×10¹³, 4×10¹³, 5×10¹³, 6×10¹³, 7×10¹³, 8×10¹³, 9×10¹³, 1×10¹⁴, 1.9×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In one embodiment, the concentration of the viral vector in the composition is 1×10¹³ VG/mL. In one embodiment, the concentration of the viral vector in the composition is 1.1×10¹² VG/mL. In one embodiment, the concentration of the viral vector in the composition is 3.7×10¹² VG/mL. In one embodiment, the concentration of the viral vector in the composition is 8×10¹¹ VG/mL. In one embodiment, the concentration of the viral vector in the composition is 2.6×10¹² VG/mL. In one embodiment, the concentration of the viral vector in the composition is 4.9×10¹² VG/mL. In one embodiment, the concentration of the viral vector in the composition is 0.8×10¹² VG/mL. In one embodiment, the concentration of the viral vector in the composition is 0.83×10¹² VG/mL. In one embodiment, the concentration of the viral vector in the composition is the maximum final dose which can be contained in a vial. In one embodiment, the concentration of the viral vector in the composition is 1.6×10¹¹ VG/mL. In one embodiment, the concentration of the viral vector in the composition is 5×10¹¹ VG/mL. In one embodiment, the concentration of the viral vector in the composition is 2.3×10¹³ VG/mL. In one embodiment, the concentration of the viral vector in the composition is 1.9×10¹⁴ VG/mL.

In one embodiment, delivery of viral vector pharmaceutical compositions in accordance with the present invention to cells of the central nervous system (e.g., parenchyma) may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 1.6×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.6×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.3×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject. In one embodiment, the concentration of the viral vector in the composition is 2.3×10¹¹ VG/subject. In one embodiment, the concentration of the viral vector in the composition is 7.2×10¹¹ VG/subject. In one embodiment, the concentration of the viral vector in the composition is 7.5×10¹¹ VG/subject. In one embodiment, the concentration of the viral vector in the composition is 1.4×10¹² VG/subject. In one embodiment, the concentration of the viral vector in the composition is 4.8×10¹² VG/subject. In one embodiment, the concentration of the viral vector in the composition is 8.8×10¹² VG/subject. In one embodiment, the concentration of the viral vector in the composition is 2.3×10¹² VG/subject. In one embodiment, the concentration of the viral vector in the composition is 2×10¹⁰ VG/subject. In one embodiment, the concentration of the viral vector in the composition is 1.6×10¹¹ VG/subject. In one embodiment, the concentration of the viral vector in the composition is 4.6×10¹¹ VG/subject.

In one embodiment, AAV particles of the present invention may be delivered as an 80 L infusion of a composition with the concentration of 5×10¹¹ vg/ml, yielding a total of 1.6×10¹¹ VG delivered to each patient.

In one embodiment, AAV particles of the present invention may be delivered as an 80 μL infusion of a composition with the concentration of 5×10¹¹ vg/ml, yielding a total of 1.6×10¹¹ VG delivered to each patient.

In one embodiment, AAV particles of the present invention may be delivered as an 80 μL infusion of a composition with the concentration of 5×10¹¹ vg/ml at a rate of 3 ul per min, yielding a total of 1.6×10¹¹ VG delivered to each patient.

In one embodiment, AAV particles of the present invention may be delivered as an 80 μL infusion of a composition with the concentration of 5×10¹¹ vg/ml at a rate of 3 ul per min, yielding a total of 1.6×10¹¹ VG delivered to each patient.

In one embodiment, the effectiveness of the dose, route of administration and/or volume of administration may be evaluated using various methods described herein such as, but not limited to, PET imaging, L-DOPA challenge test, UPDRS scores and patient diaries. As a non-limiting example, a subject may have decreased dyskinesia or periods of dyskinesia after administration of the AADC polynucleotide composition. As another non-limiting example, a subject may have a decrease in AADC Deficiency related symptoms including limited mobility and dyskinesia.

In one embodiment, a subject who may be administered a dose of the AADC polynucleotides described herein may have been previously treated with the same or similar therapeutic. In another embodiment, a subject may have been treated with a therapeutic which has been shown to reduce the symptoms of AADC Deficiency.

In one embodiment, a subject who may be administered a dose of the AADC polynucleotides described herein may have failed to derive adequate benefit from standard medical therapy. As a non-limiting example, the subject may not have responded to treatment. As another non-limiting example, a subject may have residual disability despite treatment.

In one embodiment, a subject who may be administered a dose of the AADC polynucleotides described herein may undergo testing to evaluate the levels of neurotransmitter analytes to determine the effectiveness of the dose. As a non-limiting example, CSF neurotransmitters, plasma AADC activity and/or urine VLA may be analyzed.

In one embodiment, a subject who may be administered a dose of the AADC polynucleotide described herein may be videotaped or recorded in order to monitor the progress of the subject during the course of treatment.

### Combinations

The AAV particles or viral vectors comprising the AADC polynucleotide may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses delivery of pharmaceutical, prophylactic, diagnostic, or imaging compositions in combination with agents that may improve their bioavailability, reduce and/or modify their metabolism, inhibit their excretion, and/or modify their distribution within the body.

### Delivery

In one embodiment, the viral vector comprising an AADC polynucleotide may be administered or delivered using the methods for the delivery of AAV virions described in European Patent Application No. EP1857552, the contents of which are herein incorporated by reference in their entirety.

In one embodiment, the viral vector comprising an AADC polynucleotide may be administered or delivered using the methods for delivering proteins using AAV vectors described in European Patent Application No. EP2678433, the contents of which are herein incorporated by reference in their entirety.

In one embodiment, the viral vector comprising an AADC polynucleotide may be administered or delivered using the methods for delivering DNA molecules using AAV vectors described in U.S. Pat. No. 5,858,351, the contents of which are herein incorporated by reference in their entirety.

In one embodiment, the viral vector comprising an AADC polynucleotide may be administered or delivered using the methods for delivering DNA to the bloodstream described in U.S. Pat. No. 6,211,163, the contents of which are herein incorporated by reference in their entirety.

In one embodiment, the viral vector comprising an AADC polynucleotide may be administered or delivered using the methods for delivering AAV virions described in U.S. Pat. No. 6,325,998, the contents of which are herein incorporated by reference in their entirety.

In one embodiment, the viral vector comprising an AADC polynucleotide may be administered or delivered using the methods for delivering a payload to the central nervous system described in U.S. Pat. No. 7,588,757, the contents of which are herein incorporated by reference in their entirety.

In one embodiment, the viral vector comprising an AADC polynucleotide may be administered or delivered using the methods for delivering a payload described in U.S. Pat. No. 8,283,151, the contents of which are herein incorporated by reference in their entirety.

In one embodiment, the viral vector comprising an AADC polynucleotide may be administered or delivered using the methods for delivering a payload using a glutamic acid decarboxylase (GAD) delivery vector described in International Patent Publication No. WO2001089583, the contents of which are herein incorporated by reference in their entirety.

In one embodiment, the viral vector comprising an AADC polynucleotide may be administered or delivered using the methods for delivering a payload to neural cells described in International Patent Publication No. WO2012057363, the contents of which are herein incorporated by reference in their entirety.

The AAV particle of the present invention may be administered or delivered by any of the methods described in United States Publication No. US20120220648; Clinical trial NCT01395641; Hwu, W. L., et al., 2012 Gene therapy for aromatic L-amino acid decarboxylase deficiency. Sci. Transl. Med. Vol. 4, 134ra61; Lee, N.C., et al., 2013 Treatment of congenital neurotransmitter deficiencies by intracerebral ventricular injection of an adeno-associated virus serotype 9 vector. Hum. Gen. Ther. 25:189-198; and Lee, N.C., et al., 2015 Benefits of neuronal preferential systemic gene therapy for neurotransmitter deficiency. Mol. Ther. Vol. 23(10), 1572-1581, the contents of each of which are herein incorporated by reference in their entirety.

In one embodiment, the AADC polynucleotides of the present invention may be delivered in the same manner as described in Hwu, W. L., et al., 2012 Gene therapy for aromatic L-amino acid decarboxylase deficiency. Sci. Transl. Med. Vol. 4, 134ra61, the contents of which are herein incorporated by reference in their entirety, wherein two burr holes were created on either side of midline of the skull to target the putamen in each hemisphere, ensuring the burr holes are sufficiently distant from one another in the dorsolateral direction. A guide tube was first inserted, the stylet removed and a long needle stereotactically inserted for the infusion. During the infusion the needle was slowly withdrawn so as to allow vector distribution along the length of the needle tract (6-8 mm).

### Delivery to Cells

The present disclosure provides a method of delivering to a cell or tissue any of the above-described AAV polynucleotides or AAV genomes, comprising contacting the cell or tissue with said AAV polynucleotide or AAV genomes or contacting the cell or tissue with a particle comprising said AAV polynucleotide or AAV genome, or contacting the cell or tissue with any of the described compositions, including pharmaceutical compositions. The method of delivering the AAV polynucleotide or AAV genome to a cell or tissue can be accomplished in vitro, ex vivo, or in vivo.

### Delivery to Subjects

The present disclosure additionally provides a method of delivering to a subject, including a mammalian subject, any of the above-described AAV polynucleotides or AAV genomes comprising administering to the subject said AAV polynucleotide or AAV genome, or administering to the subject a particle comprising said AAV polynucleotide or AAV genome, or administering to the subject any of the described compositions, including pharmaceutical compositions.

The pharmaceutical compositions of viral vectors described herein may be characterized by one or more of bioavailability, therapeutic window and/or volume of distribution.

### Bioavailability

Viral vectors comprising an AADC polynucleotide of the present invention, when formulated into compositions with delivery/formulation agents or vehicles as described herein, may exhibit increased bioavailability as compared to compositions lacking delivery agents as described herein. As used herein, the term “bioavailability” refers to the systemic availability of a given amount of a particular agent administered to a subject. Bioavailability may be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound may be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Cₘₐₓ values are maximum concentrations of compounds achieved in serum or plasma of a subject following administration of compounds to the subject. Cₘₐₓ values of particular compounds may be measured using methods known to those of ordinary skill in the art. As used herein, the phrases “increasing bioavailability” or “improving the pharmacokinetics,” refer to actions that may increase the systemic availability of a viral vector of the present disclosure (as measured by AUC, Cₘₐₓ, or Cₘᵢₙ) in a subject. In some embodiments, such actions may comprise co-administration with one or more delivery agents as described herein. In some embodiments, the bioavailability of viral vectors may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Therapeutic Window

Viral vectors comprising an AADC polynucleotide of the present invention, when formulated with one or more delivery agents as described herein, may exhibit increases in the therapeutic window of compound and/or composition administration as compared to the therapeutic window of viral vectors administered without one or more delivery agents as described herein. As used herein, the term “therapeutic window” refers to the range of plasma concentrations, or the range of levels of therapeutically active substance at the site of action, with a high probability of eliciting a therapeutic effect. In some embodiments, therapeutic windows of viral vectors when administered in a formulation may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Volume of Distribution

Viral vectors comprising an AADC polynucleotide of the present invention, when formulated with one or more delivery agents as described herein, may exhibit an improved volume of distribution (Vdist), e.g., reduced or targeted, relative to formulations lacking one or more delivery agents as described herein. Vdist relates the amount of an agent in the body to the concentration of the same agent in the blood or plasma. As used herein, the term “volume of distribution” refers to the fluid volume that would be required to contain the total amount of an agent in the body at the same concentration as in the blood or plasma: Vdist equals the amount of an agent in the body/concentration of the agent in blood or plasma. For example, for a 10 mg dose of a given agent and a plasma concentration of 10 mg/L, the volume of distribution would be 1 liter. The volume of distribution reflects the extent to which an agent is present in the extravascular tissue. Large volumes of distribution reflect the tendency of agents to bind to the tissue components as compared with plasma proteins. In clinical settings, Vdist may be used to determine loading doses to achieve steady state concentrations. In some embodiments, volumes of distribution of viral vector compositions of the present disclosure when co-administered with one or more delivery agents as described herein may decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Kits and Devices

### Kits

The present disclosure provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

Any of the AADC vectors, AADC constructs, AADC polynucleotides, or AADC polypeptides of the present disclosure may be comprised in a kit. In some embodiments, kits may further include reagents and/or instructions for creating and/or synthesizing compounds and/or compositions of the present invention. In some embodiments, kits may also include one or more buffers. In some embodiments, kits may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there are more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present invention, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits. In such embodiments, dye may then be resuspended in any suitable solvent, such as DMSO.

In some embodiments, kits may include instructions for employing kit components as well as the use of any other reagent not included in the kit. Instructions may include variations that may be implemented.

### Devices

In some embodiments, AADC compounds and/or AADC compositions of the present disclosure may be combined with, coated onto or embedded in a device. Devices may include, but are not limited to stents, pumps, and/or other implantable therapeutic devices. Additionally AADC compounds and/or AADC compositions may be delivered to a subject while the subject is using a compression device such as, but not limited to, a compression device to reduce the chances of deep vein thrombosis (DVT) in a subject.

In some embodiments, AADC compounds and/or AADC compositions of the present invention may be delivered using a device such as, but not limited to, a stent, a tube, a catheter, a pipe, a straw, needle and/or a duct. Methods of using these devices are described herein and are known in the art.

The present disclosure provides for devices which may incorporate viral vectors that encode one or more AADC polynucleotide payload molecules. These devices contain in a stable formulation the viral vectors which may be immediately delivered to a subject in need thereof, such as a human patient.

Devices for administration may be employed to deliver the viral vectors comprising an AADC polynucleotide of the present disclosure according to single, multi- or split-dosing regimens taught herein.

Methods and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

The polynucleotides of the present disclosure may be used in the treatment, prophylaxis, palliation or amelioration of any disease or disorder characterized by aberrant or undesired target expression.

In one embodiment, the AADC polynucleotides of the present invention may be administered to a subject using delivery systems which integrate image guided therapy and integrate imaging such as, but not limited to, laser, MRgFUS, endoscopic and robotic surgery devices.

In one embodiment, the AADC polynucleotides of the present invention may be administered to a subject using the CLEARPOINT® neuro intervention system by MRI Interventions, Inc. The CLEARPOINT® neuro intervention system may be used alone or in combination with any of the other administration methods and devices described herein. The CLEARPOINT® neuro intervention system helps to provide stereotactic guidance in the placement and operation of instruments or devices during the planning and operation of neurological procedures.

In one embodiment, the AADC polynucleotides of the present invention may be administered to a subject using the NEUROMATE® stereotactic robot system by Renishaw PLC. The NEUROMATE® system may be used alone or in combination with any of the other administration methods and devices described herein. As a non-limiting example, the NEUROMATE® system may be used with head holders, CT image localizers, frame attachments, remote controls and software.

In one embodiment, the AADC polynucleotides of the present invention may be administered to a subject using the Elekta MICRODRIVE™ device by Elekta AB. The MICRODRIVE™ device may be used alone or in combination with any of the other administration methods and devices described herein. As a non-limiting example, the MICRODRIVE™ device may be used to position electrodes (e.g., for micro electrode recording (MER), macro stimulation and deep brain stimulation (DBS) electrode implantation), implantation of catheters, tubes or DBS electrodes using cross-hair and A-P holders to verify position, biopsies, injections and aspirations, brain lesioning, endoscope guidance and GAMMA KNIFE® radiosurgery.

In one embodiment, the AADC polynucleotides of the present invention may be administered to a subject using the AXIIIS® stereotactic miniframe by MONTERIS® Medical, Inc. The AXIIIS® stereotactic miniframe may be used alone or in combination with any of the other administration methods and devices described herein. The AXIIIS® stereotactic miniframe is a trajectory alignment device which may be used for laser coagulation, biopsies, catheter placement, electrode implant, endoscopy, and clot evacuation. The miniframe allows for 360 degree interface and provides access to multiple intracranial targets with a simple adjustment. Further, the miniframe is compatible with MRI.

In one embodiment, the AADC polynucleotides of the present invention may be administered to a subject using the INTEGRA™ CRW® system by Integra LifeSciences Corporation. The INTEGRA™ CRW® system may be used alone or in combination with any of the other administration methods and devices described herein. The CRW® system may be used for various applications such as, but not limited to, stereotactic surgery, microsurgery, catheterization and biopsy. The CRW® system is designed to provide accuracy to those who use the system (e.g., thumb lock screws, Vernier scaling, double bolt fixation, and a solid frame).

In one embodiment, the AADC polynucleotides of the present invention may be administered to a subject using the EPOCH® solution system by Stereotaxis, Inc. which may include the NIOBE® ES magnetic navigation system, the VDRIVE® robotic navigation system and/or the ODYSSEY® information solution (all by Stereotaxis, Inc.). The EPOCH® solution system may be used alone or in combination with any of the other administration methods and devices described herein. As a non-limiting example, the NIOBE® ES magnetic navigation system may be used to accurately contact a subject. As another non-limiting example the NIOBE® ES magnetic system may be used with the VDRIVE® robotic navigation system to provide precise movement and stability.

In one embodiment, the AADC polynucleotides of the present invention may be administered to a subject using the STEALTHSTATION® TREON™ Navigation system from Medtronic as described in Hwu, W. L., et al., 2012. Gene therapy for aromatic L-amino acid decarboxylase deficiency. Sci. Transl. Med. Vol. 4, 134ra61. The StealthStation® TREON™ system allows for the use of pre-surgery images from X-ray, CT, MRI or ultrasound and combines them with real-time images taken by LED cameras during the surgery to assist in guiding the surgeon to the desired target site in the brain or spinal cord.

In one embodiment, the AADC polynucleotides of the present invention may be administered to a subject using a NeuroStation workstation which uses frameless stereotactic methods to provide image-guidance for applications such as, but not limited to, surgical planning, biopsies, craniotomies, endoscopy, intra-operative ultrasound and radiation therapy.

In one embodiment, the AADC polynucleotides of the present invention may be administered to a subject using a robotic stereotaxis system such as, but not limited to the device described in U.S. Pat. No. 5,078,140, the contents of which are herein incorporated by reference in their entirety. The robotic arm of the device may be used to precisely orient the surgical tools or other implements used to conduct a procedure.

In one embodiment, the AADC polynucleotides of the present invention may be administered to a subject using an automatic delivery system such as, but not limited to the device described in U.S. Pat. No. 5,865,744, the contents of which are herein incorporated by reference in their entirety. Based on the images gathered by the delivery system, the computer adjusts the administration of the needle to be the appropriate depth for the particular subject.

In one embodiment, the AADC polynucleotides of the present invention may be administered to a subject who is simultaneously using during administration, and/or uses for a period of time before and/or after administration a compression device such as, but not limited to, a compression device which reduces the chances of deep vein thrombosis (DVT) in a subject. The compression device may be used for at least 5 minutes, 15 minutes, 30 minutes, 45 minutes, 1 hour, 2 hours, 3 hours, 4 hours, 5 hours, 6 hours, 7 hours, 8 hours, or more than 8 hours before a subject is administered the AADC polynucleotides. The compression device may be used for at least 5 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks or a month after the AADC polynucleotides are administered. As a non-limiting example, the compression device is used simultaneously during the procedure of the delivery of the AADC polynucleotides. As another non-limiting example, the compression device is used before the administration of the AADC polynucleotides. As another non-limiting example, the compression device is used after administration of the AADC polynucleotides. As another non-limiting example, the compression device is used before, during and after administration of the AADC polynucleotides.

Non-limiting examples, of compression devices include ActiveCare+S.F.T. intermittent compression device, ActiveCare+S.F.T pneumatic compression device, DVTlite's Venowave, KCl system compression pump, Aircast VenaFlow system, SCD Express Compression System or Bio Compression Systems, Inc. pneumatic compression therapy equipment (e.g., the pump may be selected from Model SC-2004, Model SC-2004-FC, Model SC-3004, Model SC-3004-FC, Model SC-2008, Model SC-2008-DL, Model SC-3008-T, the BioCryo system, Model IC-BAP-DL or multi-flo DVT combo IC_1545-DL and the garment used with the pump may be a 4 chamber, 8 chamber, BioCryo, Multi-Flo or BioArterial garment).

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: The term “adeno-associated virus” or “AAV” as used herein refers to members of the dependovirus genus comprising any particle, sequence, gene, protein, or component derived therefrom. The term “AAV particle” as used herein comprises a capsid and a polynucleotide. The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions described herein may have activity and this activity may involve one or more biological events.

Administered in combination: As used herein, the term “administered in combination” or “combined administration” refers to simultaneous exposure to two or more agents (e.g., AAV) administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance (e.g., an AAV) that has activity in or on a biological system and/or organism. For instance, a substance that, when administered to an organism, has a biological effect on that organism, is considered to be biologically active. In particular embodiments, a compounds and/or compositions of the present disclosure may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bonds with each other. For example, for two 20-mers, if only two base pairs on each strand can form hydrogen bonds with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering parvovirus, e.g. an AAV and/or AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present invention, e.g., viral particles or expression vectors) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Effective amount: As used herein, the term “effective amount” of an agent is that amount sufficient to effect beneficial or desired results, for example, upon single or multiple dose administration to a subject cell, in curing, alleviating, relieving or improving one or more symptoms of a disorder and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

Engineered: As used herein, embodiments are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example a protein may contain one or more amino acids, e.g., an epitope, which interacts with an antibody, e.g., a biomolecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present disclosure (e.g., a vector, AAV particle, etc.) and a delivery agent.

Fragment. A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

Functional: As used herein, a “functional” biological molecule is a biological entity with a structure and in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984), BLASTP, BLASTN, and FASTA Altschul, S. F. et al., J. Molec. Biol., 215, 403 (1990).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Operably linked. As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Protein of interest: As used herein, the terms “proteins of interest” or “desired proteins” include those provided herein and fragments, mutants, variants, and alterations thereof.

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may therefore comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Self-complementary viral particle: As used herein, a “self-complementary viral particle” is a particle comprised of at least two components, a protein capsid and a polynucleotide sequence encoding a self-complementary genome enclosed within the capsid.

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, the subject may be an infant, neonate, or a child under the age of 12 years old. In some embodiments, the subject may be in utero.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in a 24 hr period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule.

Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral construct vector: As used herein, a “viral construct vector” is a vector which comprises one or more polynucleotide regions encoding or comprising Rep and or Cap protein.

Viral construct expression vector: As used herein, a “viral construct expression vector” is a vector which comprises one or more polynucleotide regions encoding or comprising Rep and or Cap that further comprises one or more polynucleotide regions encoding or comprising components for viral expression in a viral replication cell.

Viral genome: As used herein, a “viral genome” is a polynucleotide encoding at least one inverted terminal repeat (ITR), at least one regulatory sequence, and at least one payload. The viral genome is derived by replication of a payload construct from the payload construct expression vector. A viral genome encodes at least one copy of the payload construct.

### EQUIVALENTS AND SCOPE

Those skilled in the art will recognize, or be able to ascertain using no more than routine experimentation, many equivalents to the specific embodiments in accordance with those explicitly described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

The term “comprising” is intended to be open and permits but does not require the inclusion of additional elements or steps. When the term “comprising” is used herein, the term “consisting of” is thus also encompassed and disclosed.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Section and table headings are not intended to be limiting.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Examples

### Example 1. Design of AADC Polynucleotides

AADC polynucleotides are designed to comprise at a minimum a nucleic acid sequence encoding an AADC protein.

Once designed, the sequence is engineered or synthesized or inserted in a plasmid or vector and administered to a cell or organism. Suitable plasmids or vectors are any which transduce or transfect the target cell.

Adeno-associated viral vectors (AAV), viral particles or entire viruses may be used.

Administration results in the processing of the AADC polynucleotide to generate the AADC protein which alters the etiology of the disease being treated or ameliorated.

In one non-limiting example, plasmids containing an AADC polynucleotide of the invention are given in Table 2. These AADC polynucleotides in the table are contained in a Fastback plasmid and have a CMV promoter and encode AADC. In some embodiments the open reading frame of the AADC protein mRNA is codon optimized (e.g., codop).

TABLE 2
AADC polynucleotide containing plasmids/vectors.

| Construct | SEQ ID NO |
|---|---|
| pFB CMV hAADC-1 | 2 |
| pFB CMV hAADC-2 | 3 |
| pFB CMV hAADC-3 | 4 |
| pFB CMV hAADC-4 | 5 |
| phAADC_1k | 6 |
| phAADC_2k | 7 |
| phAADC_3k | 8 |
| phAADC_4 | 9 |
| phAADC_5k | 10 |
| phAADC_6k | 11 |
| phAADC_9k | 12 |

### Example 2. AADC Polynucleotides: ITR to ITR

AADC polynucleotides suitable for use in an AAV viral vector include those in Table 3.

Table 3 provides the ITR to ITR sequences from Table 2.

TABLE 3
ITR to ITR AADC polynucleotides

| Construct | SEQ ID NO |
|---|---|
| pFB CMV hAADC-1 (ITR to ITR) | 13 |
| pFB CMV hAADC-2 (ITR to ITR) | 14 |
| pFB CMV hAADC-3 (ITR to ITR) | 15 |
| pFB CMV hAADC-4 (ITR to ITR) | 16 |
| phAADC_1k (ITR to ITR) | 17 |
| phAADC_2k (ITR to ITR) | 18 |
| phAADC_3k (ITR to ITR) | 19 |
| phAADC_4 (ITR to ITR) | 20 |
| phAADC_5k (ITR to ITR) | 21 |
| phAADC_6k (ITR to ITR) | 22 |
| phAADC_9k (ITR to ITR) | 23 |

### Example 3. Relative to the ITR to ITR parent sequence

AADC polynucleotides are designed according to Tables 4-6. The start and stop positions given are relative to the ITR to ITR AADC polynucleotides described in Table 3. In Tables 4-6 “--” means the component is not applicable.

TABLE 4
Component modules or sequence
regions of AADC polynucleotides

| | pFB CMV | | pFB CMV | | pFB CMV | | pFB CMV | |
|---|---|---|---|---|---|---|---|---|
| | hAADC-1 | | hAADC-2 | | hAADC-3 | | hAADC-4 | |
| | (ITR to ITR) | | (ITR to ITR) | | (ITR to ITR) | | (ITR to ITR) | |
| | Start | Stop | Start | Stop | Start | Stop | Start | Stop |
| 5′ ITR | 1 | 130 | 1 | 130 | 1 | 130 | 1 | 130 |
| CMV Enhancer | 263 | 566 | 263 | 566 | 263 | 566 | 296 | 599 |
| CMV Promoter | 567 | 769 | 567 | 769 | 567 | 769 | 600 | 802 |
| ie1 exon 1 | 784 | 917 | 784 | 917 | 784 | 917 | 817 | 950 |
| ie1 intron1 | 918 | 949 | 918 | 949 | 918 | 949 | 951 | 982 |
| hBglobin | 950 | 1296 | 950 | 1296 | 950 | 1296 | 983 | 1329 |
| intron2 | | | | | | | | |
| hBglobin | 1297 | 1349 | 1297 | 1349 | 1297 | 1349 | 1330 | 1382 |
| exon 3 | | | | | | | | |
| 5′ UTR | — | — | — | — | — | — | 1398 | 1468 |
| hAADC | 1374 | 2822 | — | — | 1374 | 2822 | 1473 | 2921 |
| hAADC codop | — | — | 1374 | 2816 | — | — | — | — |
| 3′ UTR | — | — | — | — | 2823 | 3221 | 2922 | 3361 |
| hGH poly(A) | 2841 | 3317 | 2835 | 3311 | 3240 | 3716 | 3380 | 3856 |
| signal | | | | | | | | |
| 3′ ITR | 3417 | 3535 | 3416 | 3534 | 3822 | 3940 | 3961 | 4079 |

TABLE 5
Component modules or sequence
regions of AADC polynucleotides

| | hAADC_1k | | hAADC_2k | | hAADC_3k | | hAADC_4 | |
|---|---|---|---|---|---|---|---|---|
| | Start | Stop | Start | Stop | Start | Stop | Start | Stop |
| 5′ ITR | 1 | 141 | 1 | 141 | 1 | 141 | 1 | 141 |
| CMV Enhancer | 245 | 548 | 245 | 548 | 245 | 548 | 245 | 548 |
| CMV Promoter | 549 | 751 | 549 | 751 | 549 | 751 | 549 | 751 |
| ie1 exon 1 | 766 | 899 | 766 | 899 | 766 | 899 | 766 | 899 |
| ie1 intron1 | 900 | 931 | 900 | 931 | 900 | 931 | 900 | 931 |
| hBglobin | 932 | 1278 | 932 | 1278 | 932 | 1278 | 932 | 1278 |
| intron2 | | | | | | | | |
| hBglobin | 1279 | 1331 | 1279 | 1331 | 1279 | 1331 | 1279 | 1331 |
| exon 3 | | | | | | | | |
| 5′ UTR | — | — | — | — | — | — | 1347 | 3310 |
| hAADC | 1356 | 2804 | 1356 | 2804 | — | — | 1422 | 2864 |
| hAADC codop | — | — | — | — | 1356 | 2798 | — | — |
| 3′ UTR | — | — | — | — | — | — | 2865 | 3310 |
| hGH poly(A) | 2823 | 3299 | 2823 | 3299 | 2817 | 3293 | 3329 | 3805 |
| signal | | | | | | | | |
| 3′ ITR | 3357 | 3497 | 3357 | 3497 | 3351 | 3491 | 3863 | 4003 |

TABLE 6
Component modules or sequence
regions of AADC polynucleotides

| | hAADC_5k | | hAADC_6k | | hAADC_9k | |
|---|---|---|---|---|---|---|
| | Start | Stop | Start | Stop | Start | Stop |
| 5′ ITR | 1 | 145 | 1 | 141 | 1 | 130 |
| CMV Enhancer | 249 | 552 | 245 | 548 | 234 | 537 |
| CMV Promoter | 553 | 755 | 549 | 751 | 538 | 740 |
| ie1 exon 1 | 770 | 903 | 766 | 899 | 755 | 888 |
| ie1 intron1 | 904 | 935 | 900 | 931 | 889 | 920 |
| hBglobin | 936 | 1282 | 932 | 1278 | 921 | 1267 |
| intron2 | | | | | | |
| hBglobin | 1283 | 1335 | 1279 | 1331 | 1268 | 1320 |
| exon 3 | | | | | | |
| 5′ UTR | — | — | — | — | — | — |
| hAADC | | | 1356 | 2798 | 1345 | 2793 |
| hAADC codop | 1360 | 2802 | — | — | — | — |
| 3′ UTR | — | — | 2799 | 3203 | — | — |
| hGH poly(A) | 2821 | 3297 | 3222 | 3698 | 2812 | 3288 |
| signal | | | | | | |
| 3′ ITR | 3355 | 3499 | 3756 | 3896 | 3346 | 3475 |

### Example 4. AADCD Patient Identification

The present disclosure is directed to the treatment, prophylaxis, palliation and/or amelioration of AADCD and related genetic inborn errors of metabolism in an affected pediatric population. Because AADCD presents in infancy and very early childhood, the presently described method employing AAV-mediated gene therapy is particularly useful. Firstly, the BBB may not yet be fully “closed” in the young brain, and secondly, neonates and infants are likely to be seronegative for AAV antigens and/or neutralizing antibodies that would otherwise impede transduction and successful gene therapy.

For the studies described herein, a population of AADC-deficient subjects is identified. In some embodiments, the AADC-deficient subjects are rodent models for AADCD. In some embodiments, the AADC-deficient subjects are non-human primates. In some embodiments a population of AADC-deficient human pediatric patients is identified.

The diagnosis of AADCD is made through detailed clinical assessment, analysis of cerebrospinal fluid neurotransmitters, and further supportive diagnostic investigations. In AADCD, the CSF neurotransmitter profile and other observations include: Low HVA, 5-HIAA, 3-methoxy-4-hydroxyphenylglycol with raised 5-HTP, L-DOPA, and 3-ortho-methyldopa; AADC enzyme activity in plasma is very low or absent; urinary catecholamines raised (vanillactic acid, 3-orthomethyldopa) (Ng, et al., 2014, Pediatr. Drugs (2014) 16:275-291).

By way of non-limiting example, appropriate subjects for this study are male and female subjects (age range, 3 months to twelve years of age). Before the gene transfer, all of the patients are likely bedridden, and none have head control or are able to talk; they experience oculogyric crises every 2 to 3 days, and these attacks last for hours and consist of eye deviations, dystonia, irritability, hypersalivation, and biting of the tongue and lips; the patients cry in response to minimal stimulation and may display excessive sweating and unstable body temperature; additionally, they choke and regurgitate during feeding and are all underweight, if not emaciated. AADC gene mutations are identified in all patients. Several subjects bear the common IVS6+4A>T mutation, and others, the c. 1297_1298insA mutation. Brain magnetic resonance imaging (MRI) reveal normal brain structures, intact striatum, and normal myelination. The only abnormal finding may be mild cortical atrophy. The lack of structural brain anomalies in these patients is in marked contrast to their severe physical limitations, therefore making them ideal candidates for this new treatment approach. Patients are followed for more than two years after gene transfer (See Brun et al., 2010, Neurology, 75(1):64-71; Hwu, W. L., et al., 2012. Gene therapy for aromatic L-amino acid decarboxylase deficiency. Sci. Transl. Med. Vol. 4, 134ra61).

For example, a 24-week study is designed to evaluate the safety and tolerability of infusion of the herein disclosed AADC polynucleotide-containing recombinant adeno-associated virus (AAV) vector compositions in human patients diagnosed with AADCD. Patients are evaluated preoperatively and monthly postoperatively for six months, using multiple measures, including the Global Systonia Scale (GDS) (see Comella, et al., 2003, Movement Disorders, 18(3):303-312), motor state diaries, and laboratory tests. Using diaries that separate the day into half-hour segments, the caregivers of the patients will record their mobility during the four days before admission and for another four days at six months after admission to the study site. The patient caregivers are trained to rate subject's condition as sleeping, immobile, mobile without troublesome dyskinesias, or mobile with troublesome dyskinesias. The total number of hours spent in each of these categories is calculated, and the differences between the baseline and the six-month scores are compared between the groups. The short-duration response to levodopa is evaluated at baseline and 6 months after gene transfer; subjects take 100 mg of levodopa orally with 25 mg benserazide after 20 hours without dopaminergic medication. Motor symptoms based on GDS and plasma levodopa concentrations are assessed at baseline and 30 minutes, 1, 2, 3, and 4 hours after levodopa intake (See, for example, Muramatsu, et al., 2010, “A phase I study of aromatic L-amino acid decarboxylase gene therapy for Parkinson's disease.” Mol. Ther. 18:1731-1735).

### Example 5. Administration of AADC Polynucleotide Compositions to Patients for Gene Therapy

AADC polynucleotide-containing recombinant AAV vector compositions are infused into the substantia nigra, and in particular, the substantia nigra pars compacta (SNpc) and ventral tegmental area (VTA) of patients having AADCD and identified as qualified for treatment according to the parameters set forth in Example 4.

One method of administration contemplated for use in the methods described herein is real-time convection-enhanced delivery (RCD) of AADC polynucleotide-containing AAV vector compositions by co-infusion of gadoteridol (a magnetic resonance (MR) contrast agent) and T1 or T2 magnetic resonance imaging (MRI), which can predict areas of subsequent AADC gene expression. As described in Richardson, et al., 2011, the accuracy of cannula placement and initial infusate distribution may be safely determined by saline infusion without significantly altering the subsequent distribution of the tracer agent (Richardson, et al., 2011, Neurosurgery, 69(1): 154-163). T2 RCD provides detection of intraparenchymal convection-enhanced delivery in the uninjured brain and may predict subsequent distribution of a transgene after viral vector infusion. Subjects undergo saline infusion/T2 acquisition, immediately followed by gadoteridol infusion/T1 acquisition in the putamen and brainstem. Distribution volumes and spatial patterns are analyzed. Gadoteridol and AAV-encoded AADC are co-infused under alternating T2/T1 acquisition in the thalamus, and hyperintense areas are compared with areas of subsequent transgene expression. Ratios of distribution volume to infusion volume are expected to be similar between saline and gadoteridol RCD. Spatial overlap should correlate well between T2 and T1 images. The second infusate will follow a spatiotemporal pattern similar to that of the first, filling the target area before developing extra-target distribution. Areas of AADC expression should correlate well with areas of both T1 and T2 hyperintensity observed during RCD (Richardson, et al., 2011, Neurosurgery, 69(1):154-163).

Convection-enhanced delivery (CED) of macromolecules directly into the brain parenchyma has been known for over two decades. CED is a term that denotes the use of a pressure gradient to generate bulk flow within the brain parenchyma, i.e. convection of macromolecules within the interstitial fluid driven by infusing a solution through a cannula placed directly in the targeted structure. This method allows therapeutic agents to be homogenously distributed through large volumes of brain tissue by bypassing the blood brain barrier and surpassing simple diffusion (Richardson, et al., 2011, Stereotact. Funct. Neurosurg. 89:141-151).

Salegio, et al. recently demonstrated the distribution of nanoparticles of different sizes, including micelles (˜15 nm in size), AAV (˜20-25 nm) and liposomes (˜65 nm), within the CNS of rodents and NHPs (Salegio et al., 2014, Frontiers in Neuroanatomy, vol. 8, article 9: pp. 1-8). Simple injections cannot engage the perivascular system, and specialized infusion cannulae are required, enabling constant pressures to be exerted at the tip of the cannula such that the interstitial hydrostatic pressure is exceeded and infusate can flow out into the tissue. Simple needles generate significant reflux; thus, reflux-resistant cannulas have been developed to counter this tendency. The advent of platforms for MRI-guided convection-enhanced infusions further refined understanding of the mechanics of perivascular flow, and it was demonstrated that perivascular distribution of liposomes was linear with respect to time, the slope of the curve was increased in myelinated regions, and cessation of infusion prevented further expansion in the volume of distribution. (Richardson, et al., 2011, Stereotact. Funct. Neurosurg. 89:141-151; Salegio et al., 2014, Frontiers in Neuroanatomy, vol. 8, article 9: pp. 1-8).

Intraparenchymal rAAV injections are known to result in robust but relatively local transduction. Such local delivery methods are advantageous when attempting gene therapy for neurological disorders that result from neuropathology that is localized to a specific anatomical region or anatomical circuitry such as in the case of Parkinson's disease. However, in treatments requiring more widespread CNS transduction, intraparenchymal injections are impractical. Treatment of neurological disorders attributable to inborn errors of metabolism and/or single-gene defects, or those that affect motor neurons of the spinal cord can require transduction of large proportions of the brain or spinal cord, respectively. Development of less invasive trans-BBB delivery methods for vectors is an extremely important endeavor. Numerous attempts to use molecules that are known to interact with various active transport mechanisms (probably receptor-mediated) to convey proteins across the BBB have been reported with varying results. Given the large number of AAV serotypes available, one or more serotypes may bind a cell-entry receptor capable of transporting the AAV capsid across the BBB (Manfredsson, et al., 2009, “AAV9: a potential blood-brain barrier buster.” Molecular Therapy 17(3):403-405).

### Vector and Stereotaxic Infusion

A stereotactic approach may be used to surgically deliver the AAV-AADC vector. Although individuals with AADC deficiency lack epinephrine and norepinephrine, these patients should maintain stable blood pressure and heart rates during the surgery. There should be no notable intracerebral hemorrhages in the postoperative computed tomography (CT) or MRI scans. The needle tracts, as shown on the MRI scans, should show accurate injection into the substantia nigra pars compacta (SNpc) and ventral tegmental area (VTA). The patients will be discharged from the hospital about one week after the surgery (Hwu, W. L., et al., 2012. Gene therapy for aromatic L-amino acid decarboxylase deficiency. Sci. Transl. Med. Vol. 4, 134ra61).

Subjects of treatment receive the AAV-vector composition vector, safely delivered to substantia nigra pars compacta (SNpc) and ventral tegmental area (VTA) via bilateral infusions, or alternatively, intrastriatally (into the caudate nucleus and putamen), or into the subthalamic nucleus (STN), for example optionally using the FDA-approved SmartFlow neuroventricular cannula (SurgiVision, Inc.) specifically designed for clinical application, with or without the aid of the ClearPoint system to help treating neurosurgeon(s) target and observe the delivery of the therapeutic agent in the brain (See, for example, San Sebastian, et al., 2014, Mol. Ther. Methods Clin. Dev. 3: 14049; See, for example, Feng and Maguire-Zeiss, 2010, CNS Drugs 24(3):177-192).

For example, during the surgery, two target points are determined in the substantia nigra pars compacta (SNpc) and ventral tegmental area (VTA) that are sufficiently separated from each other in dorsolateral directions and identified on a magnetic resonance image. One burr hole is trepanned in each side of the cranial bone, through which the vector is injected into the two target points via the two-track insertion route. The AAV-vector-containing solution is prepared to a concentration of 1.5×10¹² vector genome/ml, and 50 l per point of the solution is injected at 1 al/min; each patient receives 3×10¹¹ vector genome the AAV-vector construct.

Neutralizing antibody titers against AAV2 are determined by measuring 3-galactosidase activities in HEK293 cells transduced with 5×10³ vector genome/cell of AAV2 vectors expressing β-galactosidase in various dilutions of sera.

### PET

The AADC expression level in the substantia nigra are assessed on PET imaging with FMT six days before surgery and at one- and six-months after gene transfer. All patients cease taking dopaminergic medications 18 hours before PET and take 2.5 mg/kg of carbidopa orally one hour before FMT injection. Subsequently, 0.12 mCi/kg of FMT in saline is infused into an antecubital vein, and a 90-minute dynamic acquisition sequence is obtained. The PET and magnetic resonance imaging data are co-registered with a fusion processing program (Syntegra; Philips, Amsterdam, The Netherlands) to produce the fusion images. Radioactivities within volumes of interest drawn in the nigrostriatal pathway are calculated between 80 and 90 minutes after tracer injection. A change in nigrostriatal pathway FMT uptake from baseline to 24 weeks is assessed using the substantia nigra to striatal ratio of radioactivities.

### Statistical Analysis

Values at baseline and 6 months after gene transfer are compared using Student's t-test (paired analyses). A two-sided P value <0.05 is taken to indicate significant differences. Two-way analysis of variance with Bonferroni correction of P values is used for the short-duration response to levodopa. (See, for example, Muramatsu, et al., 2010, “A phase I study of aromatic L-amino acid decarboxylase gene therapy for Parkinson's disease.” Mol. Ther. 18:1731-1735).

Safety and tolerability of bilateral administration of AAV-vector compositions using real-time image-guided infusion into the brains of AADCD subjects may be monitored for up to or after 9 months post-surgery. Broad coverage of targeted areas (substantia nigra pars compacta (SNpc) and ventral tegmental area (VTA) and widespread AADC protein distribution in the striatum should be achieved without inducing any adverse effects.

### Changes in Growth and Motor Skills.

The patients should gain weight and exhibit improvement in their motor scores after gene transfer, within a year, post-treatment. Weight will be measured at 3 to 6 months after gene transfer. All patients initially should have raw scores of zero on the Alberta Infant Motor Scale (AIMS) and very low raw scores for the Peabody Developmental Motor Scale, Second Edition (PDMS-II). After the gene transfer, all of the patients should show continuous increases in their raw scores on these two scales, which indicates that their motor functions have improved. The Comprehensive Developmental Inventory for Infants and Toddlers (CDIIT) covers both cognition and motor development. All of the patients should show low raw CDIIT scores before gene transfer, and the subsequent increase in scores demonstrate improvement in both motor and cognitive functions.

Subjective Improvements after Gene Transfer

To document the symptoms that are more difficult to quantify, parents of the patients are asked to fill out a questionnaire at the end of the study. The symptoms of the oculogyric crises should lessen, and eye deviations and sleep disruptions, for example, are some mild symptoms of the oculogyric crises that may remain after gene therapy. Subjects may experience increased emotional stability, and/or some improvements in sweating and hyperthermia (a common manifestation of body temperature instability in hot weather). There should be no detectable abnormality in heart rate variability as assessed by 24-hour Holter monitoring either before or after gene transfer. Before gene therapy, patients that were bedridden and showed little spontaneous movement may exhibit less severe ptosis (drooping of the upper eyelid) one to two weeks after the gene transfer. According to previous studies, dyskinesia may occur one month after gene transfer, but upon observation of a decrease in dyskinesia, motor development should start (Hwu, W. L., et al., 2012. Gene therapy for aromatic L-amino acid decarboxylase deficiency. Sci. Transl. Med. Vol. 4, 134ra61). Subjects may exhibit increased head control after three months, sitting with support after six to nine months, sitting up from the prone position after thirteen months, and holding toys and standing with support sixteen months after the gene transfer, for example. Anti-AAV2 antibodies should be negative in the patients before gene therapy, and the titers may increase slightly after gene transfer.

PET scans and CSF analyses

PET scans and CSF analyses are completed for the treated patients. Six months after gene transfer, PET scans should reveal that uptake of 6-[18F] fluorodopa (FDOPA) increase from baseline in the combined (right and left) treatment sites. The CSF analysis should reveal increases in the levels of homovanillic acid (HVA, a metabolite of dopamine) and 5-hydroxyindoleacetic acid (HIAA, a metabolite of serotonin). However, the levels of L-DOPA and 3-O-methyldopa may remain elevated (Hwu, W. L., et al., 2012. Gene therapy for aromatic L-amino acid decarboxylase deficiency. Sci. Transl. Med. Vol. 4, 134ra61).

### Example 6. Administration of AADC Polynucleotides

AADC polynucleotide-containing recombinant AAV vector compositions are infused into the putamen of patients having AADCD and identified as qualified for treatment according to the parameters set forth in Example 4 using the administration methods described in Example 5. The dose, number of patients and volume are outlined in Table 7.

TABLE 7
Study Design

| | Number of | | | |
|---|---|---|---|---|
| Study No. | Patients | Dose | Volume | |
| 1 | 6 | 3 × 10¹¹ vg | 100 | ul per putamen |
| 2 | 6 | 9 × 10¹¹ vg | 300 | ul per putamen |
| 3 | 10 | 2.3 × 10¹¹ vg | 100 | ul per putamen |
| 4 | 10 | 7.5 × 10¹¹ vg | 100 | ul per putamen |
| 5 | 5 | 7.5 × 10¹¹ vg | 450 | ul per putamen |
| 6 | Up to 20 | 1.4 × 10¹² vg | Up to 900 | ul per putamen |
| 7 | Up to 20 | 4.8 × 10¹² vg | Up to 900 | ul per putamen |
| 8 | Up to 20 | 8.8 × 10¹² vg | Up to 900 | ul per putamen |

During the course of the study the safety and tolerability of the infusion of the AADC polynucleotide-containing recombinant adeno-associated virus (AAV) vector compositions in human patients diagnosed with AADCD is evaluated. Patients are evaluated preoperatively and monthly postoperatively for six months, using multiple measures, including the Global Systonia Scale (GDS) (see Comella, et al., 2003, Movement Disorders, 18(3):303-312), L-DOPA challenge test, UPDRS scores, motor state diaries, and laboratory tests. Using diaries that separate the day into half-hour segments, the caregivers of the patients will record their mobility during the four days before admission and for another four days at six months after admission to the study site. The patient caregivers are trained to rate subject's condition as sleeping, immobile, mobile without troublesome dyskinesias, or mobile with troublesome dyskinesias. The total number of hours spent in each of these categories is calculated, and the differences between the baseline and the six-month scores are compared between the groups. The short-duration response to levodopa is evaluated at baseline and 6 months after gene transfer; subjects take 100 mg of levodopa orally with 25 mg benserazide after 20 hours without dopaminergic medication. Motor symptoms based on GDS and plasma levodopa concentrations are assessed at baseline and 30 minutes, 1, 2, 3, and 4 hours after levodopa intake (See, for example, Muramatsu, et al., 2010, “A phase I study of aromatic L-amino acid decarboxylase gene therapy for Parkinson's disease.” Mol. Ther. 18:1731-1735).

### Example 7. In Vivo Administration of AADC Polynucleotides

Two AADC polynucleotide-containing recombinant AAV vector compositions (SEQ ID NO: 17 and 19), a control and a standard were administered to rats (n=5) by bilateral intrastriation (10 ul/side) at a dose level of 2×10¹² vg/ml. The expression of AADC in rat striatum was determined by ELISA after 4 and 8 weeks. Variation was seen between the animals and the hemispheres due to variable delivery between the infusion sites. Both SEQ ID NO: 10 and 12 expressed AADC, but SEQ ID NO: 12 showed up to 200% increase of expression as compared to the standard construct.

### Example 8. Dose Response Study of AADC Polynucleotides

Compositions of AADC polynucleotide-containing recombinant AAV vectors (SEQ ID NO: 17) at five different dose levels ranging from 1×10¹¹ vg/ml to 1×10¹³ vg/ml and a control are administered to 6-OHDA lesioned rats. The behavioral response to low-dose levodopa administration is quantified before and after (week 3 and 4) delivery of the composition. 5 weeks after dosing, necropsy is conducted and the AADC enzymatic activity is measured in ex vivo striatal tissue assay and the distribution of AADC in the brain is determined by immunohistochemical (IHC) staining.

### Example 9. Effect of Empty Particles on Intrastriatal Transduction

Adult rats (n=6) were administered varying ratios of full:empty vector particles at: 0% full, 50% full, 85% full or 99% full. AADC polynucleotide-containing recombinant AAV vector (SEQ ID NO: 10) at a constant dose and volume (5 ul and 1×10 vg) was administered intrastriatally. The rats were evaluated 4 weeks after administration. The low vector dose resulted in limited AADC vector expression. The volume of distribution for the particles is shown in Table 8 (ELISA) and the striatal levels of AADC expression is shown in Table 9 (Histology).

TABLE 8
Volume of Distribution

| % Ratio of full AAV2-AADC | Approximate Volume of Distribution |
|---|---|
| particle to empty capsids | (mm³) |
| 50:50 | 2 |
| 70:30 | 2.4 |
| 85:15 | 2.5 |
| 100:0 | 3 |

TABLE 9
Striatal Levels

| % full of AAV2-AADC particles | AADC pg/ug protein |
|---|---|
| 0 | 0.4 |
| 52 | 1.1 |
| 58 | 1.7 |
| 83 | 2 |
| >99 | 2.1 |

Distribution was comparable for all groups. Relatively low vector dose resulted in limited AADC expression. There was also a trend to lower AADC expression levels with >30% empty particles.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

We claim:

1. A method of treating AADC deficiency or a related childhood monoamine neurotransmitter disorder by administering a composition comprising at least one AAV particle to a substantia nigra pars compacta of a subject in need of treatment or amelioration of said disorder, wherein said at least one AAV particle comprises an AADC polynucleotide selected from the group consisting of SEQ ID NOs 2-24 or variants having at least 95% identity thereto.

2. The method of claim 1, wherein the AADC polynucleotide has at least 95% identity to any of the sequences selected from SEQ ID NOs 2-24.

3. The method of claim 1, wherein the AADC polynucleotide has at least 99% identity to any of the sequences selected from SEQ ID NOs 2-24.

4. The method of claim 1, wherein the AAV particle comprises a capsid serotype selected from the group consisting of AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9.

5. The method of claim 4, wherein the capsid serotype is selected from the group consisting of AAV6, AAVrh10, AAV9 (hu14), AAV-DJ and AAV9.47.

6. The method of claim 1, wherein the AAV particle comprising an AADC polynucleotide further comprises a promoter region, 5′ UTR, 3′ UTR and a poly(A) signal.

7. The method of claim 6, wherein the AADC polynucleotide further comprising at least one 5′ ITR and one 3′ ITR.

8. The method of claim 7, wherein one or more of the 5′ ITRs are located at the 5′ end of the promoter region and one or more of the 3′ ITRs are located at the 3′ end of the poly(A) signal.

9. The method of claim 7, wherein the promoter region comprises an enhancer element, a promoter element, a first exon region, a first intron region, a second intron region and a second exon region.

10. The method of claim 9, wherein the enhancer element and the promoter element are derived from CMV.

11. The method of claim 10, wherein the first exon region is ie1 exon 1 or fragments thereof, the first intron region is ie1 intron 1 or fragments thereof, the second intron region is hBglobin intron 2 or fragments thereof and the second exon region is hBglobin exon 3 or fragments thereof.

12. The method of claim 6, wherein the poly(A) signal is derived from human growth hormone.

13. The method of claim 1, wherein the subject is a human between 2 and 8 years old.

14. The method of claim 1, wherein the ratio of AAV particles comprising the AADC polynucleotide and AAV particles without the AADC polynucleotide in the composition is at least 70:30.

15. The method of claim 14, wherein the ratio of AAV viral particles comprising the AADC polynucleotide to AAV viral particles without the AADC polynucleotide in the composition is at least 85:15.

16. The method of claim 14, wherein the ratio of AAV viral particles comprising the AADC polynucleotide to AAV viral particles without the AADC polynucleotide in the composition is at least 100:0.

17. The method of claim 1, wherein the subject uses a compression device during the contacting of the subject with the composition and wherein the compression device reduces the chances of deep vein thrombosis in the subject.

18. A method of treating AADC deficiency or a related childhood monoamine neurotransmitter disorder by administering a composition comprising at least one AAV particle to a putamen of a subject in need of treatment or amelioration of said disorder, wherein said at least one AAV particle comprises an AADC polynucleotide selected from the group consisting of SEQ ID NOs 2-24 or variants having at least 95% identity thereto.

19. The method of claim 18, wherein the ratio of AAV particles comprising the AADC polynucleotide and AAV particles without the AADC polynucleotide in the composition is at least 70:30.

20. The method of claim 19, wherein the ratio of AAV viral particles comprising the AADC polynucleotide to AAV viral particles without the AADC polynucleotide in the composition is at least 85:15.

21. The method of claim 19, wherein the ratio of AAV viral particles comprising the AADC polynucleotide to AAV viral particles without the AADC polynucleotide in the composition is at least 100:0.

22. The method of claim 18, wherein the subject uses a compression device during the contacting of the subject with the composition and wherein the compression device reduces the chances of deep vein thrombosis in the subject.

23. The method of claim 18, wherein the at least one AAV particle comprises a capsid serotype selected from the group consisting of AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9.

24. The method of claim 23, wherein the capsid serotype is selected from the group consisting of AAV6, AAVrh10, AAV9 (hu14), AAV-DJ and AAV9.47.

25. The method of claim 18, wherein the at least one AAV particle comprising an AADC polynucleotide further comprises a promoter region, 5′ UTR, 3′ UTR and a poly(A) signal.

26. The method of claim 25, wherein the AADC polynucleotide further comprising at least one 5′ ITR and one 3′ ITR.

27. The method of claim 26, wherein one or more of the 5′ ITRs are located at the 5′ end of the promoter region and one or more of the 3′ ITRs are located at the 3′ end of the poly(A) signal.

28. The method of claim 26, wherein the promoter region comprises an enhancer element, a promoter element, a first exon region, a first intron region, a second intron region and a second exon region.

29. The method of claim 28, wherein the enhancer element and the promoter element are derived from CMV.

30. The method of claim 29, wherein the first exon region is ie1 exon 1 or fragments thereof, the first intron region is ie1 intron 1 or fragments thereof, the second intron region is hBglobin intron 2 or fragments thereof and the second exon region is hBglobin exon 3 or fragments thereof.

31. The method of claim 25, wherein the poly(A) signal is derived from human growth hormone.

32. A method of improving the sleep-wake cycle of a subject comprising contacting said subject with a composition comprising at least one AAV particle to a putamen of a subject in need of treatment or amelioration of said disorder, wherein said at least one AAV particle comprises an AADC polynucleotide selected from the group consisting of SEQ ID NOs 2-24 or variants having at least 95% identity thereto.

33. The method of claim 32, wherein the at least one AAV particle comprises a capsid serotype selected from the group consisting of AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9.

34. The method of claim 33, wherein the capsid serotype is selected from the group consisting of AAV6, AAVrh10, AAV9 (hu14), AAV-DJ and AAV9.47.

35. The method of claim 32, wherein the AAV particle comprising an AADC polynucleotide further comprises a promoter region, 5′ UTR, 3′ UTR and a poly(A) signal.

36. The method of claim 35, wherein the AADC polynucleotide further comprising at least one 5′ ITR and one 3′ ITR.

37. The method of claim 36, wherein one or more of the 5′ ITRs are located at the 5′ end of the promoter region and one or more of the 3′ ITRs are located at the 3′ end of the poly(A) signal.

38. The method of claim 36, wherein the promoter region comprises an enhancer element, a promoter element, a first exon region, a first intron region, a second intron region and a second exon region.

39. The method of claim 38, wherein the enhancer element and the promoter element are derived from CMV.

40. The method of claim 39, wherein the first exon region is ie1 exon 1 or fragments thereof, the first intron region is ie1 intron 1 or fragments thereof, the second intron region is hBglobin intron 2 or fragments thereof and the second exon region is hBglobin exon 3 or fragments thereof.

41. The method of claim 35, wherein the poly(A) signal is derived from human growth hormone.

42. A method of treating a sleep disorder of a subject comprising contacting said subject with a composition comprising at least one AAV particle to a putamen of a subject in need of treatment or amelioration of said disorder, wherein said at least one AAV particle comprises an AADC polynucleotide selected from the group consisting of SEQ ID NOs 2-24 or variants having at least 95% identity thereto.

43. The method of claim 42, wherein the at least one AAV particle comprises a capsid serotype selected from the group consisting of AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9.

44. The method of claim 43, wherein the capsid serotype is selected from the group consisting of AAV6, AAVrh10, AAV9 (hu14), AAV-DJ and AAV9.47.

45. The method of claim 42, wherein the at least one AAV particle comprising an AADC polynucleotide further comprises a promoter region, 5′ UTR, 3′ UTR and a poly(A) signal.

46. The method of claim 45, wherein the AADC polynucleotide further comprising at least one 5′ ITR and one 3′ ITR.

47. The method of claim 46, wherein one or more of the 5′ ITRs are located at the 5′ end of the promoter region and one or more of the 3′ ITRs are located at the 3′ end of the poly(A) signal.

48. The method of claim 46, wherein the promoter region comprises an enhancer element, a promoter element, a first exon region, a first intron region, a second intron region and a second exon region.

49. The method of claim 48, wherein the enhancer element and the promoter element are derived from CMV.

50. The method of claim 49, wherein the first exon region is ie1 exon 1 or fragments thereof, the first intron region is ie1 intron 1 or fragments thereof, the second intron region is hBglobin intron 2 or fragments thereof and the second exon region is hBglobin exon 3 or fragments thereof.

51. The method of claim 45, wherein the poly(A) signal is derived from human growth hormone.
